(19) United States
(12) Patent Application Publication
(10) Pub. No.: US 2005/0227270 A1
(43) Pub. Date: Oct. 13, 2005

(54) Alternatively spliced isoforms of sodium channel, voltage gated, type XI, alpha (SCN11A)

(76) Inventors: Christopher K. Raymond, Seattle, WA (US); Philip W. Garrett-Engele, Seattle, WA (US)

Correspondence Address: R. Douglas Bradley;Merck & Co., Inc., Patent Department RY60-30, P.O. Box 2000, Rahway, NJ, 07065-0907, US

(21) Appl. No.: 11/082,454

(22) Filed: Mar. 16, 2005

Related U.S. Application Data
(60) Provisional application No. 60/553,721, filed on Mar. 16, 2004.
(60) Provisional application No. 60/565,088, filed on Apr. 22, 2004.

(51) Int. Cl. C12Q 1/68; C07H 21/04; C12N 15/09; C07K 14/705

(52) U.S. Cl. 435/6; 435/69.1; 435/320.1; 435/325; 530/350; 536/23.5

(57) ABSTRACT

The present invention features nucleic acids and polypeptides encoding novel splice variant isoforms of sodium channel, voltage gated, type XI, alpha (SCN11A). The polynucleotide sequence of SCN11Asv1 is provided by SEQ ID NO 5. The amino acid sequence of SCN11Asv1 is provided by SEQ ID NO 6. The polynucleotide sequence of SCN11Asv2 is provided by SEQ ID NO 7. The amino acid sequence of SCN11Asv2 is provided by SEQ ID NO 8. The present invention also provides methods for using SCN11Asv1 or SCN11Asv2 polynucleotides and proteins to screen for compounds that bind to SCN11Asv1 or SCN11Asv2, respectively.

This application claims priority to U.S. Provisional Patent Application Ser. No. 60/553,721 filed on Mar. 16, 2004, and U.S. Provisional Patent Application Ser. No. 60/565,088 filed on Apr. 22, 2004, each of which is incorporated by reference herein in its entirety.

### BACKGROUND OF THE INVENTION

The references cited herein are not admitted to be prior art to the claimed invention.

Human and mouse genomes contain at least ten related sodium channel genes encoding proteins having highly conserved sequences (reviewed in Meisler et al., 2001, Neuroscientist 7(2): 136-145; Wood and Baker, 2001, Curr. Opin. in Pharm. 1: 17-21; Goldin et al., 2000, Neuron 28: 365-368). Sodium channels mediate the influx of sodium ions in response to changes in membrane potential in electrically excitable cells such as neurons and muscle (Catterall, W. A., 1992, Physiol. Rev. 72, (Suppl.) S15-S48). Sodium channels consist of an about 260 kilo Dalton (kD) pore-forming alpha (α) subunit and auxiliary beta (β) subunits that modify the kinetics of voltage dependent channel gating (reviewed in Catterall, W. A., 2000, Neuron 26: 13-25; Goldin et al., 2000; Wood and Baker, 2001). The alpha subunit of a voltage gated sodium channel has four homologous domains (Domains I, II, III and IV), each containing six α-helical transmembrane segments (reviewed in Catterall, W. A., 2000; Goldin et al., 2000; Wood and Baker, 2001). While the β-subunits are required for normal kinetics and gating of the channel, expression of the α-subunit is alone sufficient for sodium currents in Xenopus oocytes (Noda et al., 1986, Nature 322: 213-216; Akopian et al., 1996, Nature 379: 257-262; reviewed in Catterall, W. A., 2000).

Although sodium channels have highly conserved protein sequences, they have diverged with respect to tissue-specific and temporal gene expression. Four genes—SCN1A (Naᵥ1.5), SCN2A (Naᵥ1.2), SCN3A (Naᵥ1.3), and SCN11A (Naᵥ1.6)—are highly expressed in the central nervous system. Three genes—SCN9A (Naᵥ1.7), SCN10A (Naᵥ1.8), and SCN11A (Naᵥ1.9)—are predominantly expressed in the peripheral nervous system. And three genes—SCN4A (Naᵥ1.4), SCN5A (Naᵥ1.5), and SCN7A (Naₓ)—are expressed in muscle (reviewed in Meisler et al., 2000).

Mutations in sodium channel genes have been implicated in several human disorders. Two mutations that cause generalized epilepsy with febrile seizures have been identified in the SCN1A (Naᵥ1.1) gene (Escayg et al., 2000, Nat. Genet. 24: 343-345). Brugada syndrome, characterized by ventricular fibrillation, heart failure and sudden death, is associated with mutations in the cardiac sodium channel SCN5A (Naᵥ1.5) (Chen et al., 1998, Nature 392: 293-296; Baroudi et al., 2000, FEBS Lett. 467: 12-16). Mutations in SCN5A have also been associated with the cardiac arrhythmia of Long-QT syndrome type 3 (Wang et al., 1995, Cell 80: 805-811). Neurological disorders including ataxia, dystonia, severe muscle weakness, and paralysis are associated with mutations in SCN8A (Burgess et al., 1995, Nat. Genet. 10: 461-465; Sidman et al., 1979, Ann. N.Y. Acad. Sci. 317: 497-505; Sprunger et al., 1999, Hum. Mol. Genet. 8: 471-479; Kohrman et al., 1996, J. Neurosci. 16: 5993-5999; Repentigny et al., 2001, Hum. Mol. Genet. 10: 1819-1827; reviewed in Meisler et al., 1997, Ann. Med. 29: 569-574). Mutations in the skeletal muscle channel gene SCN4A (Naᵥ1.4) have been implicated in hyperkalaemic periodic paralysis (Bulman, D. E., 1997, Hum. Mol. Gen. 6: 1679-1685; Rojas et al., 1999, Am. J. Physiol. 276: C259-C266). In the mouse, mutations in SCN10A (Naᵥ1.8) have been linked to reduced pain sensitivity (Akopian et al., 1999, Nat. Neurosci. 2: 541-548; Laird et al., 2002, J. Neurosci. 22(19): 8352-8356).

The SCN11A sodium channel alpha subunit gene has been identified in several organisms (reviewed in Dib-Hajj et al., 2002, TRENDS in Neurosci 25: 253-259). The rat SCN11A (NaN) gene encodes a 1,765 amino acid protein (Dib-Hajj et al, 1998, Proc. Natl. Acad. Sci USA 95: 8963-8968). Rat SCN11A mRNA is expressed preferentially in small sensory neurons of dorsal root ganglia and trigeminal ganglia (Dib-Hajj et al, 1998, Proc. Natl. Acad. Sci USA 95: 8963-8968).

The mouse SCN11A gene also encodes a 1,765 amino acid protein (Dib-Hajj et al., 1999, Genomics 59: 309-318; Ogata et al., 2000, Biochemical and Biophysical Research Communications 267: 271-277). Mouse SCN11A mRNA is expressed as a 6.0-kilobase transcript that is expressed predominantly in testes, trigeminal ganglia, dorsal root ganglia, spinal cord, ovary, uterus, and the small intestine (Ogata et al., 2000, Biochemical and Biophysical Research Communications 267: 271-277). In situ hybridization experiments indicated that mouse SCN11A mRNA is expressed in dorsal root ganglia, spinal cord, uterus, testis, ovary, placenta, and small intestine (Ogata et al., 2000, Biochemical and Biophysical Research Communications 267: 271-277).

The human SCN11A gene encodes a 1,791 amino acid protein that shares several conserved domains with other sodium channel alpha subunits including four internal repeat domains (each having six putative alpha-helical transmembrane segments and two pore-lining segments) and multiple putative sites for cAMP-dependent phosphorylation (Dib-Hajj et al., 1999, FEBS Lett. 462: 117-120; Jeong et al., 2000, Biochemical and Biophysical Research Communications 267: 262-270). Human SCN11A mRNA is expressed predominantly in olfactory bulb, hippocampus, cerebellar cortex, spinal cord, spleen, small intestine, and placenta, but is also expressed in the dorsal root ganglia (Jeong et al., 2000, Biochemical and Biophysical Research Communications 267: 262-270). Jeong et al have observed two human SCN11A mRNA transcripts including a 6.5-kilobase transcript which encodes a complete alpha subunit as well as a 5/7-kilobase transcript that encodes a shorter alpha subunit in which Domain IV and the C-terminal region are truncated (Jeong et al., 2000, Biochemical and Biophysical Research Communications 267: 262-270).

The human, mouse and rat SCN11A protein sequences share a high degree of similarity with one another. Human SCN11A protein shares 73% similarity with rat SCN11A protein (Jeong et al., 2000, Biochemical and Biophysical Research Communications 267: 262-270). Mouse SCN11A protein shares 88% amino acid identity with rat SCN11A (Dib-Hajj et al., 1999, Genomics 59: 309-318; Ogata et al., 2000, Biochemical and Biophysical Research Communications 267: 271-277).

Although gating of sodium channels is believed to result from changes in membrane potential, data from Blum et al., suggest that gating of SCN11A is mediated by ligand binding rather than by voltage (Blum et al., 2002, Nature 419: 687-693). Membrane depolarization evoked by brain-derived neurotrophic factor (BDNF) is mediated by a BDNF-sensing channel complex that requires the combined contribution of the receptor tyrosine kinase TrkB and the SCN11A (Naᵥ1.9) sodium channel (Blum et al., 2002, Nature 419: 687-693). Consistent with this model, SCN11A (Naᵥ1.9) antisense oligonucleotides selectively abolished the expression of BDNF evoked Na+ currents in both SH-SY5Y cells and hippocampal neurons (Blum et al., 2002, Nature 419: 687-693). This mechanistic link between SCN11A and BDNF, which is released presynaptically as well as postsynaptically in the central nervous system and is implicated in long term potentiation and possible long term memory, suggests that Na+ channels may play a role in synaptically-induced cellular plasticity (Blum et al., 2002, Nature 419: 687-693; reviewed in Delmas and Coste, 2003, TRENDS in Neurosci 26: 55-57).

Sodium channels expressed in sensory neurons are thought to play a crucial role in chronic pain neuropathies. After axonal injury, abnormal excitability of dorsal root ganglia neurons is associated with an increased density of sodium channels (Matzner and Devor, 1994, J. Neurophysiol. 72: 349-359; Zhang et al., 1997, J. Neurophysiol. 78: 2790-2794). Furthermore, experimental and clinical observations indicate that sodium channel blockers have efficacy in neuropathic pain (Chabal et al., 1989, Pain 38: 333-338; Devor et al., 1992 Pain 48: 261-268; Omana-Zapata et al., 1997, Brain Res. 771: 228-237; reviewed in Nitu et al., 2003, Expert Opin. Investig. Drugs 12: 545-559). SCN11A/NaN sodium channel currents are significantly attenuated in peripherally axotomized dorsal root ganglion neurons; this attenuation of rat SCN11A/NaN sodium channel currents could lead to hyper-excitability in these cells which may result in pain (Cummins and Waxman, 1997, J. Neurosci. 17: 3503-3514; Sleeper et al., 2000, J. Neurosci. 20: 7279-7289; reviewed in Dib-Hajj et al., 2002, TRENDS in Neurosci. 25: 253-259). Consistent with this model, a down regulation of rat SCN11A/NaN and SNS/SCN10A channels and their currents in dorsal root ganglion neurons was observed in the chronic construction injury model of neuropathic pain (Dib-Hajj, 1999, Pain 83: 591-600).

Sodium channel activity can be modified by several small molecules and peptides. The pore blockers tetrodotoxin and saxitoxin bind to sodium channels with varying affinities, depending on the amino acid sequence of these channels at key structural positions (reviewed in Catterall, W. A., 2000). For example, tetrodotoxin resistant (TTX-R) channels contain cysteine or serine residues in the SS2 segment of Domain I (Akopian, et al., 1996, Nature 379: 257-262), whereas an aromatic residue is located in tetrodotoxin sensitive (TTX-S) channels (Satin et al., 1992, Science 256: 1202-1205). Likewise, cadmium is a high-affinity blocker of cardiac sodium channels but not of brain or skeletal muscle sodium channels due to amino acid variations in these channels in Domain I (Backx et al., 1992, Science 257: 248-251; Satin et al., 1992, Science 256: 1202-1205). Sodium channel blocking drugs of diverse structure have been used as anti-arrhythmic drugs and anti-convulsants (reviewed in Catterall, W. A., 2000). Peptides with sequences corresponding to the intracellular loop connecting Domains III and IV of the sodium channel a subunit can also be used as pore blockers and can restore activation to sodium channels that are defective in inactivation (Eaholtz et al., 1994, Neuron 12: 1041-1048). Other neurotoxins such as α-scorpion toxins and sea anemone toxins uncouple sodium channel activation from inactivation by preventing the normal gating movement of the extracellular end of the IVS4 segment (Rogers et al., 1996, J. Biol. Chem. 271: 15950-15962; Sheets et al., 1999, Biophys. J. 77: 747-757).

A serine residue in the SS2 segment of Domain I is predicted to confer tetrodotoxin resistance to the rat, mouse, and human SCN11A sodium channels (Dib-Hajj et al., 1998, Proc. Natl. Acad. Sci USA 95: 8963-8968; Dib-Hajj et al., 1999, Genomics 59: 309-318; Ogata et al., 2000, Biochemical and Biophysical Research Communications 267: 271-277; Jeong et al., 2000, Biochemical and Biophysical Research Communications 267: 262-270). Consistent with this prediction, expression of rat SCN11A (NaN/SNS2) in HEK293T cells produced a tetrodotoxin resistant sodium current (Tate el al, 1998, Nature Neurosci 1: 653-655).

Significantly, cockroach SCN8A splice variants differ from one another with respect to their gating properties and sensitivity to deltamethrin, a pyrethroid insecticide (Tan, et al., 2002, J. Neurosci. 22(13): 5300-5309). As these examples illustrate, the effectiveness of small molecule effectors on sodium channels is largely dependent on the specific amino acid sequence and structure of the sodium channel. It is therefore likely that these small molecules will modulate the activity of splice variants with alternative structures and sequences differently than the reference protein.

Because of the multiple therapeutic values of drugs targeting sodium channels, including SCN11A, there is a need in the art for compounds that selectively bind to isoforms of SCN11A. The present invention is directed towards novel SCN11A isoforms (SCN11Asv1 and SCN11Asv2) and uses thereof.

### BRIEF DESCRIPTION OF THE FIGURES

FIG. 1A illustrates the exon structure of human SCN11A mRNA corresponding to the known reference form of SCN11A mRNA (labeled AF188679) and the exon structure corresponding to the inventive splice variant transcripts (labeled SCN11Asv1 and SCN11Asv2). FIG. 1B depicts the nucleotide sequences of the exon junctions resulting from the splicing of exon 15 to exon 17 in the case of SCN11Asv1 mRNA [SEQ ID NO 1], and the splicing of exon 6 to intron 6A [SEQ ID NO 2], and intron 6A to exon 7 [SEQ ID NO 3], where intron 6A is defined as the portion of intron 6 consisting of the polynucleotides 5′ AGTCTCTTAGCAGC AAGGGAGGACTTCATCCATCCAGGGTGCTCCTGTGGGCAGATGCATTTCCTAGAGAATTGCC TCATTCTTTGGCTTCCTATGGAGAAG 3′ [SEQ ID NO 4], in the case of SCN11Asv2 mRNA. In FIG. 1B, in the case of the SCN11Asv1 splice junction sequence [SEQ ID NO 1], the nucleotides shown in italics represent the 20 nucleotides at the 3′ end of exon 15 and the nucleotides shown in underline represent the 20 nucleotides at the 5′ end of exon 17. In FIG. 1B, in the case of the SCN11Asv2, exon 6-intron 6A splice junction sequence [SEQ ID NO 2], the nucleotides shown in italics represent the 20 nucleotides at the 3′ end of exon 6 and the nucleotides shown in underline represent the 20 nucleotides at the 5′ end of intron 6A; and in the case of the SCN11Asv2, intron 6A-exon 7 splice junction sequence SEQ ID NO 3, the nucleotides shown in italics represent the 20 nucleotides at the 3′ end of intron 6A and the nucleotides shown in underline represent the 20 nucleotides at the 5′ end of exon 7.

### SUMMARY OF THE INVENTION

RT-PCR and DNA sequence analysis, and real-time quantitative PCR have been used to identify and confirm the presence of novel splice variants of human SCN11A mRNA. More specifically, the present invention features polynucleotides encoding different protein isoforms of SCN11A. A polynucleotide sequence encoding SCN11Asv1 is provided by SEQ ID NO 5. An amino acid sequence for SCN11Asv1 is provided by SEQ ID NO 6. A polynucleotide sequence encoding SCN11Asv2 is provided by SEQ ID NO 7. An amino acid sequence for SCN11Asv2 is provided by SEQ ID NO 8.

Thus, a first aspect of the present invention describes a purified SCN11Asv1 encoding nucleic acid and a purified SCN11Asv2 encoding nucleic acid. The SCN11Asv1 encoding nucleic acid comprises SEQ ID NO 5 or the complement thereof. The SCN11Asv2 encoding nucleic acid comprises SEQ ID NO 7 or the complement thereof. Reference to the presence of one region does not indicate that another region is not present. For example, in different embodiments the inventive nucleic acid can comprise, consist, or consist essentially of an encoding nucleic acid sequence of SEQ ID NO 5, or can comprise, consist, or consist essentially of the nucleic acid sequence of SEQ ID NO 7.

Another aspect of the present invention describes a purified SCN11Asv1 polypeptide that can comprise, consist or consist essentially of the amino acid sequence of SEQ ID NO 6. An additional aspect describes a purified SCN11Asv2 polypeptide that can comprise, consist, or consist essentially of the amino acid sequence of SEQ ID NO 8.

Another aspect of the present invention describes expression vectors. In one embodiment of the invention, the inventive expression vector comprises a nucleotide sequence encoding a polypeptide comprising, consisting, or consisting essentially of SEQ ID NO 6, wherein the nucleotide sequence is transcriptionally coupled to an exogenous promoter. In another embodiment, the inventive expression vector comprises a nucleotide sequence encoding a polypeptide comprising, consisting, or consisting essentially of SEQ ID NO 8, wherein the nucleotide sequence is transcriptionally coupled to an exogenous promoter.

Alternatively, the nucleotide sequence comprises, consists, or consists essentially of SEQ ID NO 5, and is transcriptionally coupled to an exogenous promoter. In another embodiment, the nucleotide sequence comprises, consists, or consists essentially of SEQ ID NO 7, and is transcriptionally coupled to an exogenous promoter.

Another aspect of the present invention describes recombinant cells comprising expression vectors comprising, consisting, or consisting essentially of the above-described sequences and the promoter is recognized by an RNA polymerase present in the cell. Another aspect of the present invention describes a recombinant cell made by a process comprising the step of introducing into the cell an expression vector comprising a nucleotide sequence comprising, consisting, or consisting essentially of SEQ ID NO 5 or SEQ ID NO 7, or a nucleotide sequence encoding a polypeptide comprising, consisting, or consisting essentially of an amino acid sequence of SEQ ID NO 6 or SEQ ID NO 8, wherein the nucleotide sequence is transcriptionally coupled to an exogenous promoter. The expression vector can be used to insert recombinant nucleic acid into the host genome or can exist as an autonomous piece of nucleic acid.

Another aspect of the present invention describes a method of producing SCN11Asv1 or SCN11Asv2 polypeptide comprising SEQ ID NO 6 or SEQ ID NO 8 respectively. The method involves the step of growing a recombinant cell containing an inventive expression vector under conditions wherein the polypeptide is expressed from the expression vector.

Another aspect of the present invention features a purified antibody preparation comprising an antibody that binds selectively to SCN11Asv1 as compared to one or more sodium channel isoform polypeptides that are not SCN11Asv1. In another embodiment, a purified antibody preparation is provided comprising antibody that binds selectively to SCN11Asv2 as compared to one or more sodium channel isoform polypeptides that are not SCN11Asv2.

Another aspect of the present invention provides a method of screening for a compound that binds to SCN11Asv1, SCN11Asv2, or fragments thereof. In one embodiment, the method comprises the steps of: (a) expressing a polypeptide comprising the amino acid sequence of SEQ ID NO 6 or a fragment thereof from recombinant nucleic acid; (b) providing to said polypeptide a labeled SCN11A ligand that binds to said polypeptide and a test preparation comprising one or more test compounds; (c) and measuring the effect of said test preparation on binding of said test preparation to said polypeptide comprising SEQ ID NO 6. Alternatively, this method could be performed using SEQ ID NO 8, instead of SEQ ID NO 6.

In another embodiment of the method, a compound is identified that binds selectively to SCN11Asv1 polypeptide as compared to one or more sodium channel isoform polypeptides that are not SCN11Asv1. This method comprises the steps of: providing a SCN11Asv1 polypeptide comprising SEQ ID NO 6; providing a sodium channel isoform polypeptide that is not SCN11Asv1; contacting said SCN11Asv1 polypeptide and said sodium channel isoform polypeptide that is not SCN11Asv1 with a test preparation comprising one or more test compounds; and determining the binding of said test preparation to said SCN11Asv1 polypeptide and to said sodium channel isoform polypeptide that is not SCN11Asv1, wherein a test preparation that binds to said SCN11Asv1 polypeptide but does not bind to said sodium channel isoform polypeptide that is not SCN11Asv1 contains a compound that selectively binds said SCN11Asv1 polypeptide. Alternatively, the same method can be performed using SCN11Asv2 polypeptide comprising, consisting, or consisting essentially of SEQ ID NO 8.

In another embodiment of the invention, a method is provided for screening for a compound able to bind to or interact with a SCN11Asv1 protein or a fragment thereof comprising the steps of: expressing a SCN11Asv1 polypeptide comprising SEQ ID NO 6 or a fragment thereof from a recombinant nucleic acid; providing to said polypeptide a labeled SCN11A ligand that binds to said polypeptide and a test preparation comprising one or more compounds; and measuring the effect of said test preparation on binding of said labeled SCN11A ligand to said polypeptide, wherein a test preparation that alters the binding of said labeled SCN11A ligand to said polypeptide contains a compound that binds to or interacts with said polypeptide. In an alternative embodiment, the method is performed using SCN11Asv2 polypeptide comprising, consisting, or consisting essentially of SEQ ID NO 8 or a fragment thereof.

Another aspect of the present invention provides a method of screening for a compound that binds to one or more sodium channel isoform polypeptides that are not SCN11Asv1. This method comprises the steps of: providing a SCN11Asv1 polypeptide comprising SEQ ID NO 6; providing a sodium channel isoform polypeptide that is not SCN11Asv1; contacting said SCN11Asv1 polypeptide and sodium channel isoform polypeptide that is not SCN11Asv1 with a test preparation comprising one or more test compounds; and determining the binding of said test preparation to said SCN11Asv1 polypeptide and to said sodium channel isoform polypeptide that is not SCN11Asv1, wherein a test preparation that binds to said sodium channel isoform polypeptide that is not SCN11Asv1 but not to said SCN11Asv1 polypeptide contains a compound that selectively binds said sodium channel isoform polypeptide. Alternatively, the same method can be performed using SCN11Asv2 polypeptide comprising, consisting, or consisting essentially of SEQ ID NO 8.

Other features and advantages of the present invention are apparent from the additional descriptions provided herein, including the different examples. The provided examples illustrate different components and methodology useful in practicing the present invention. The examples do not limit the claimed invention. Based on the present disclosure the skilled artisan can identify and employ other components and methodology useful for practicing the present invention.

### DEFINITIONS

Unless defined otherwise, all technical and scientific terms used herein have the meaning commonly understood by one of ordinary skill in the art to which this invention belongs.

As used herein, “SCN11A” refers to a sodium channel, voltage gated, type XI, alpha (AAF17480). In contrast, reference to an SCN11A isoform includes AAF17480 and other polypeptide isoform variants of SCN11A.

As used herein, “SCN11Asv1 ” and “SCN11Asv2” refer to splice variant isoforms of human SCN11A protein, wherein the splice variants have the amino acid sequence set forth in SEQ ID NO 6 (for SCN11Asv1) and SEQ ID NO 8 (for SCN11Asv2).

As used herein, “SCN11A” refers to polynucleotides encoding SCN11A.

As used herein, “SCN11Asv1” refers to polynucleotides that are identical to SCN11A encoding polynucleotides, except that the sequences represented by exon 16 of the SCN11A messenger RNA is not present in SCN11Asv1. As used herein, “SCN11Asv2” refers to polynucleotides that are identical to SCN11A encoding polynucleotides, except that the sequences represented by a portion of intron 6 of the SCN11A messenger RNA are retained in SCN11Asv2. “Intron 6A” refers to the polynucleotides encoding the portion of intron 6 retained in SCN11Asv2. The polynucleotide sequence of intron 6 is set forth in SEQ ID NO 4.

As used herein, “SCN11Asv1” refers to polynucleotides encoding SCN11Asv1 having an amino acid sequence set forth in SEQ ID NO 6. As used herein, “SCN11Asv2” refers to polynucleotides encoding SCN11Asv2 having an amino acid sequence set forth in SEQ ID NO 8.

As used herein, a “sodium channel isoform” is any isoform of any sodium channel from any organism, including but not limited to human SCN1A (Naᵥ1.1), SCN2A (Naᵥ1.2), SCN3A (Naᵥ1.3), SCN4A (Naᵥ1.4), SCN5A (Naᵥ1.5), SCN6A/SCN7A (Naₓ), SCN11A (Naᵥ1.6), SCN9A (Naᵥ1.7), SCN10A (Naᵥ1.8), and SCN11A (Naᵥ1.9).

As used herein, an “isolated nucleic acid” is a nucleic acid molecule that exists in a physical form that is nonidentical to any nucleic acid molecule of identical sequence as found in nature; “isolated” does not require, although it does not prohibit, that the nucleic acid so described has itself been physically removed from its native environment. For example, a nucleic acid can be said to be “isolated” when it includes nucleotides and/or internucleoside bonds not found in nature. When instead composed of natural nucleosides in phosphodiester linkage, a nucleic acid can be said to be “isolated” when it exists at a purity not found in nature, where purity can be adjudged with respect to the presence of nucleic acids of other sequence, with respect to the presence of proteins, with respect to the presence of lipids, or with respect to the presence of any other component of a biological cell, or when the nucleic acid lacks sequence that flanks an otherwise identical sequence in an organism's genome, or when the nucleic acid possesses sequence not identically present in nature. As so defined, “isolated nucleic acid” includes nucleic acids integrated into a host cell chromosome at a heterologous site, recombinant fusions of a native fragment to a heterologous sequence, recombinant vectors present as episomes or as integrated into a host cell chromosome.

A “purified nucleic acid” represents at least 10% of the total nucleic acid present in a sample or preparation. In preferred embodiments, the purified nucleic acid represents at least about 50%, at least about 75%, or at least about 95% of the total nucleic acid in a isolated nucleic acid sample or preparation. Reference to “purified nucleic acid” does not require that the nucleic acid has undergone any purification and may include, for example, chemically synthesized nucleic acid that has not been purified.

The phrases “isolated protein”, “isolated polypeptide”, “isolated peptide” and “isolated oligopeptide” refer to a protein (or respectively to a polypeptide, peptide, or oligopeptide) that is nonidentical to any protein molecule of identical amino acid sequence as found in nature; “isolated” does not require, although it does not prohibit, that the protein so described has itself been physically removed from its native environment. For example, a protein can be said to be “isolated” when it includes amino acid analogues or derivatives not found in nature, or includes linkages other than standard peptide bonds. When instead composed entirely of natural amino acids linked by peptide bonds, a protein can be said to be “isolated” when it exists at a purity not found in nature—where purity can be adjudged with respect to the presence of proteins of other sequence, with respect to the presence of non-protein compounds, such as nucleic acids, lipids, or other components of a biological cell, or when it exists in a composition not found in nature, such as in a host cell that does not naturally express that protein.

As used herein, a “purified polypeptide” (equally, a purified protein, peptide, or oligopeptide) represents at least 10% of the total protein present in a sample or preparation, as measured on a weight basis with respect to total protein in a composition. In preferred embodiments, the purified polypeptide represents at least about 50%, at least about 75%, or at least about 95% of the total protein in a sample or preparation. A “substantially purified protein” (equally, a substantially purified polypeptide, peptide, or oligopeptide) is an isolated protein, as above described, present at a concentration of at least 70%, as measured on a weight basis with respect to total protein in a composition. Reference to “purified polypeptide” does not require that the polypeptide has undergone any purification and may include, for example, chemically synthesized polypeptide that has not been purified.

As used herein, the term “antibody” refers to a polypeptide, at least a portion of which is encoded by at least one immunoglobulin gene, or fragment thereof, and that can bind specifically to a desired target molecule. The term includes naturally-occurring forms, as well as fragments and derivatives. Fragments within the scope of the term “antibody” include those produced by digestion with various proteases, those produced by chemical cleavage and/or chemical dissociation, and those produced recombinantly, so long as the fragment remains capable of specific binding to a target molecule. Among such fragments are Fab, Fab′, Fv, F(ab)′₂, and single chain Fv (scFv) fragments. Derivatives within the scope of the term include antibodies (or fragments thereof) that have been modified in sequence, but remain capable of specific binding to a target molecule, including: interspecies chimeric and humanized antibodies; antibody fusions; heteromeric antibody complexes and antibody fusions, such as diabodies (bispecific antibodies), single-chain diabodies, and intrabodies (see, e.g., Marasco (ed.), Intracellular Antibodies: Research and Disease Applications, Springer-Verlag New York, Inc. (1998) (ISBN: 3540641513). As used herein, antibodies can be produced by any known technique, including harvest from cell culture of native B lymphocytes, harvest from culture of hybridomas, recombinant expression systems, and phage display.

As used herein, a “purified antibody preparation” is a preparation where at least 10% of the antibodies present bind to the target ligand. In preferred embodiments, antibodies binding to the target ligand represent at least about 50%, at least about 75%, or at least about 95% of the total antibodies present. Reference to “purified antibody preparation” does not require that the antibodies in the preparation have undergone any purification.

As used herein, “specific binding” refers to the ability of two molecular species concurrently present in a heterogeneous (inhomogeneous) sample to bind to one another in preference to binding to other molecular species in the sample. Typically, a specific binding interaction will discriminate over adventitious binding interactions in the reaction by at least two-fold, more typically by at least 10-fold, often at least 100-fold; when used to detect analyte, specific binding is sufficiently discriminatory when determinative of the presence of the analyte in a heterogeneous (inhomogeneous) sample. Typically, the affinity or avidity of a specific binding reaction is least about 1 μM.

The term “antisense”, as used herein, refers to a nucleic acid molecule sufficiently complementary in sequence, and sufficiently long in that complementary sequence, as to hybridize under intracellular conditions to (i) a target mRNA transcript or (ii) the genomic DNA strand complementary to that transcribed to produce the target mRNA transcript.

The term “subject”, as used herein refers to an organism and to cells or tissues derived therefrom. For example the organism may be an animal, including but not limited to animals such as cows, pigs, horses, chickens, cats, dogs, etc., and is usually a mammal, and most commonly human.

### DETAILED DESCRIPTION OF THE INVENTION

This section presents a detailed description of the present invention and its applications. This description is by way of several exemplary illustrations, in increasing detail and specificity, of the general methods of this invention. These examples are non-limiting, and related variants that will be apparent to one of skill in the art are intended to be encompassed by the appended claims.

The present invention relates to the nucleic acid sequences encoding human SCN11Asv1 and SCN11Asv2 that are alternatively spliced isoforms of SCN11A, and to the amino acid sequences encoding these proteins. SEQ ID NO 5 and SEQ ID NO 7 are polynucleotide sequences representing exemplary open reading frames that encode the SCN11Asv1 and SCN11Asv2 proteins, respectively. SEQ ID NO 6 shows the polypeptide sequence of SCN11Asv1. SEQ ID NO 8 shows the polypeptide sequence of SCN11Asv2.

SCN11Asv1 and SCN11Asv2 polynucleotide sequences encoding SCN11Asv1 and SCN11Asv2 proteins, as exemplified and enabled herein include a number of specific, substantial and credible utilities. For example, SCN11Asv1 and SCN11Asv2 encoding nucleic acids were identified in an mRNA sample obtained from a human source (see Example 1). Such nucleic acids can be used as hybridization probes to distinguish between cells that produce SCN11Asv1 and SCN11Asv2 transcripts from human or non-human cells (including bacteria) that do not produce such transcripts. Similarly, antibodies specific for SCN11Asv1 or SCN11Asv2 can be used to distinguish between cells that express SCN11Asv1 or SCN11Asv2 from human or non-human cells (including bacteria) that do not express SCN11Asv1 or SCN11Asv2.

The importance of SCN11A as a drug target for neurological disorders including acute or chronic pain, or other hyperexcitabililty phenomena, is evidenced by attenuation of SCN11A sodium channel currents after axonal injury, which is associated with abnormal excitability of dorsal root ganglions (Cummins and Waxman, 1997, J. Neurosci. 17: 3503-3514; Sleeper et al., 2000, J. Neurosci. 20: 7279-7289; reviewed in Dib-Hajj et al., 2002, TRENDS in Neurosci. 25: 253-259). The hyperexcitability in these cells may result in pain. Given the potential importance of SCN11A activity to the therapeutic management of acute and/or chronic pain pathologies, it is of value to identify SCN11A isoforms and identify SCN11A-ligand compounds that are isoform specific, as well as compounds that are effective ligands for two or more different SCN11A isoforms or sodium channel isoforms. In particular, it may be important to identify compounds that are effective inhibitors of a specific SCN11A isoform activity, yet do not bind to or interact with a plurality of different SCN11A isoforms or sodium channel isoforms. Compounds that bind to or interact with multiple SCN11A isoforms may require higher drug doses to saturate multiple SCN11A-isoform binding sites and thereby result in a greater likelihood of secondary non-therapeutic side effects. Furthermore, biological effects could also be caused by the interaction of a drug with the SCN11Asv1 or SCN11Asv2 isoforms specifically. For the foregoing reasons, SCN11Asv1 and SCN11Asv2 proteins represent useful compound binding targets and have utility in the identification of new SCN11A-ligands and sodium channel isoform-ligands exhibiting a preferred specificity profile and having greater efficacy for their intended use.

In some embodiments, SCN11Asv1 and SCN11Asv2 activity is modulated by a ligand compound to achieve one or more of the following: prevent or reduce the risk of occurrence, or recurrence of neurological disorders including acute and chronic pain.

Compounds modulating SCN11Asv1 or SCN11Asv2 include agonists, antagonists, and allosteric modulators. While not wishing to be limited to any particular theory of therapeutic efficacy, generally, but not always, SCN11Asv1 or SCN11Asv2 compounds will be used to modulate the activity of the SCN11A voltage gated sodium channel. These compounds may act as pore blockers and inhibit the passage of sodium ions across the cellular membrane. Compounds that uncouple sodium channel activation from inactivation by preventing the normal gating movement of the extracellular end of the IVS4 segment may also be utilized (Rogers et al., 1996; Sheets et al., 1999). Peptides with sequences corresponding to the intracellular loop connecting Domains III and IV may act as pore blockers and may restore activation to sodium channels that are defective in inactivation (Eaholtz et al., 1994). Sodium channel blocking drugs have been used as anti-arrhythmic drugs and anti-convulsants (reviewed in Catterall, W. A., 2000). Therefore, agents that modulate SCN11A activity may be used to achieve a therapeutic benefit for any disease or condition due to, or exacerbated by, SCN11A ion channel activity.

SCN11Asv1 or SCN11Asv2 activity can also be affected by modulating the cellular abundance of transcripts encoding SCN11Asv1 or SCN11Asv2, respectively. Compounds modulating the abundance of transcripts encoding SCN11Asv1 or SCN11Asv2 include a cloned polynucleotide encoding SCN11Asv1 or SCN11Asv2, respectively, that can express SCN11Asv1 or SCN11Asv2 in vivo, antisense nucleic acids targeted to SCN11Asv1 or SCN11Asv2 transcripts, enzymatic nucleic acids, such as ribozymes, and RNAi nucleic acids, such as shRNAs or siRNAs,,targeted to SCN11Asv1 or SCN11Asv2 transcripts.

In some embodiments, SCN11Asv1 or SCN11Asv2 activity is modulated to achieve a therapeutic effect upon diseases in which regulation of SCN11A is desirable. For example, neurological disorders such as chronic and acute pain may be treated by modulating SCN11Asv1 or SCN11Asv2 sodium channel activity.

SCN11Asv1 and SCN11Asv2 Nucleic Acids

SCN11Asv1 nucleic acids contain regions that encode for polypeptides comprising, consisting, or consisting essentially of SEQ ID NO 6. SCN11Asv2 nucleic acids contain regions that encode for polypeptides comprising, consisting, or consisting essentially or SEQ ID NO 8. The SCN11Asv1 and SCN11Asv2 nucleic acids have a variety of uses, such as use as a hybridization probe or PCR primer to identify the presence of SCN11Asv1 or SCN11Asv2 nucleic acids, respectively; use as a hybridization probe or PCR primer to identify nucleic acids encoding for proteins related to SCN11Asv1 or SCN11Asv2, respectively; and/or use for recombinant expression of SCN11Asv1 or SCN11Asv2 polypeptides. In particular, SCN11Asv1 polynucleotides do not have the polynucleotide region that consists of exon 16 of the SCN11A gene. SCN11Asv2 polynucleotides have an additional polynucleotide region that comprises intron 6A [SEQ ID NO 4] of the SCN11A gene.

Regions in SCN11Asv1 or SCN11Asv2 nucleic acid that do not encode for SCN11Asv1 or SCN11Asv2, or are not found in SEQ ID NO 5, or SEQ ID NO 7, if present, are preferably chosen to achieve a particular purpose. Examples of additional regions that can be used to achieve a particular purpose include: a stop codon that is effective at protein synthesis termination; capture regions that can be used as part of an ELISA sandwich assay; reporter regions that can be probed to indicate the presence of the nucleic acid; expression vector regions; and regions encoding for other polypeptides.

The guidance provided in the present application can be used to obtain the nucleic acid sequence encoding SCN11Asv1 or SCN11Asv2 related proteins from different sources. Obtaining nucleic acids encoding SCN11Asv1 or SCN11Asv2 related proteins from different sources is facilitated by using sets of degenerative probes and primers and the proper selection of hybridization conditions. Sets of degenerative probes and primers are produced taking into account the degeneracy of the genetic code. Adjusting hybridization conditions is useful for controlling probe or primer specificity to allow for hybridization to nucleic acids having similar sequences.

Techniques employed for hybridization detection and PCR cloning are well known in the art. Nucleic acid detection techniques are described, for example, in Sambrook, et al., in Molecular Cloning, A Laboratory Manual, 2^{nd} Edition, Cold Spring Harbor Laboratory Press, 1989. PCR cloning techniques are described, for example, in White, Methods in Molecular Cloning, volume 67, Humana Press, 1997.

SCN11Asv1 or SCN11Asv2 probes and primers can be used to screen nucleic acid libraries containing, for example, cDNA. Such libraries are commercially available, and can be produced using techniques such as those described in Ausubel, Current Protocols in Molecular Biology, John Wiley, 1987-1998.

Starting with a particular amino acid sequence and the known degeneracy of the genetic code, a large number of different encoding nucleic acid sequences can be obtained. The degeneracy of the genetic code arises because almost all amino acids are encoded for by different combinations of nucleotide triplets or “codons”. The translation of a particular codon into a particular amino acid is well known in the art (see, e.g., Lewin GENES IV, p. 119, Oxford University Press, 1990). Amino acids are encoded for by codons as follows:
A=Ala=Alanine: codons GCA, GCC, GCG, GCU
C=Cys=Cysteine: codons UGC, UGU
D=Asp=Aspartic acid: codons GAC, GAU
E=Glu=Glutamic acid: codons GAA, GAG
F=Phe=Phenylalanine: codons UWC, UUU
G=Gly=Glycine: codons GGA, GGC, GGG, GGU
H=His=Histidine: codons CAC, CAU
I=Ile=Isoleucine: codons AUA, AUC, AUU
K=Lys=Lysine: codons AAA, AAG
L=Leu=Leucine: codons UUA, UUG, CUA, CUC, CUG, CUU
M=Met=Methionine: codon AUG
N=Asn=Asparagine: codons AAC, AAU
P=Pro=Proline: codons CCA, CCC, CCG, CCU
Q=Gln=Glutamine: codons CAA, CAG
R=Arg=Arginine: codons AGA, AGG, CGA, CGC, CGG, CGU
S=Ser=Serine: codons AGC, AGU, UCA, UCC, UCG, UCU
T=Thr=Threonine: codons ACA, ACC, ACG, ACU
V=Val=Valine: codons GUA, GUC, GUG, GUU
W=Trp=Tryptophan: codon UGG
Y=Tyr=Tyrosine: codons UAC, UAU

Nucleic acid having a desired sequence can be synthesized using chemical and biochemical techniques. Examples of chemical techniques are described in Ausubel, Current Protocols in Molecular Biology, John Wiley, 1987-1998, and Sambrook et al., in Molecular Cloning, A Laboratory Manual, 2^{nd} Edition, Cold Spring Harbor Laboratory Press, 1989. In addition, long polynucleotides of a specified nucleotide sequence can be ordered from commercial vendors, such as Blue Heron Biotechnology, Inc. (Bothell, Wash.).

Biochemical synthesis techniques involve the use of a nucleic acid template and appropriate enzymes such as DNA and/or RNA polymerases. Examples of such techniques include in vitro amplification techniques such as PCR and transcription based amplification, and in vivo nucleic acid replication. Examples of suitable techniques are provided by Ausubel, Current Protocols in Molecular Biology, John Wiley, 1987-1998, Sambrook et al., in Molecular Cloning, A Laboratory Manual, 2^{nd} Edition, Cold Spring Harbor Laboratory Press, 1989, and U.S. Pat. No. 5,480,784.

SCN11Asv1 and SCN11Asv2 Probes

Probes for SCN11Asv1 or SCN11Asv2 contain a region that can specifically hybridize to SCN11Asv1 or SCN11Asv2 target nucleic acids, respectively, under appropriate hybridization conditions and can distinguish SCN11Asv1 or SCN11Asv2 nucleic acids from each other and from non-target nucleic acids, in particular SCN11A polynucleotides containing exon 16 and SCN11A polynucleotides lacking intron 6A [SEQ ID NO 4]. Probes for SCN11Asv1 or SCN11Asv2 can also contain nucleic acid regions that are not complementary to SCN11Asv1 or SCN11Asv2 nucleic acids.

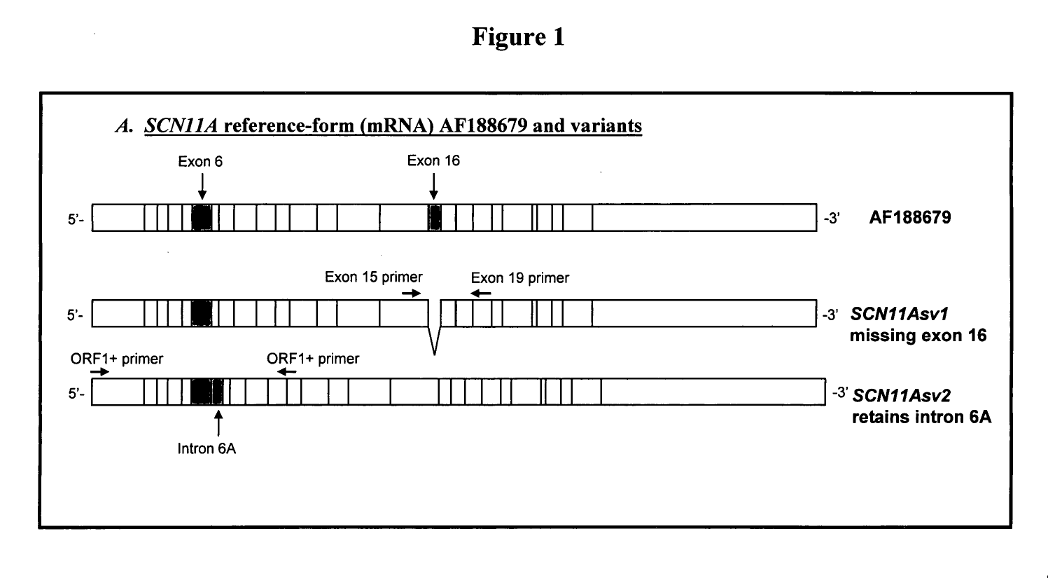

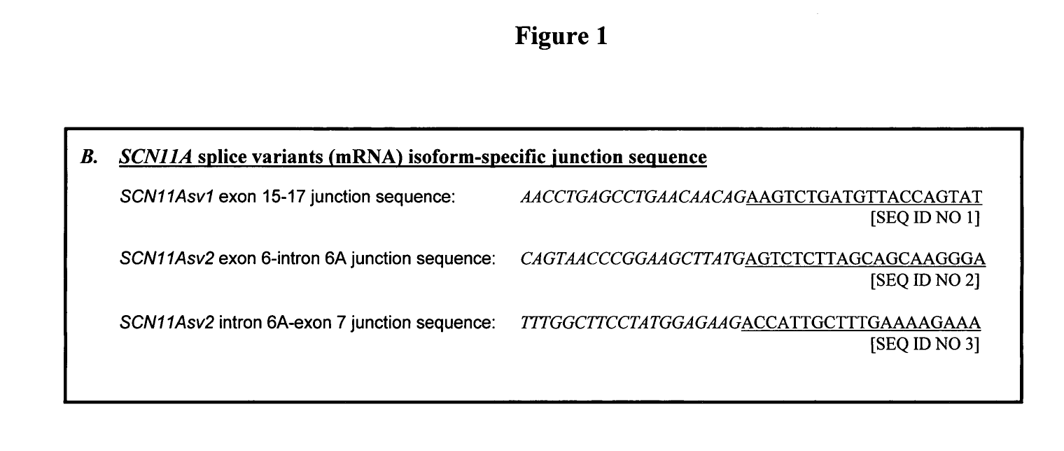

In embodiments where, for example, SCN11Asv1 or SCN11Asv2 polynucleotide probes are used in hybridization assays to specifically detect the presence of SCN11Asv1 or SCN11Asv2 polynucleotides in samples, the SCN11Asv1 or SCN11Asv2 polynucleotides comprise at least 20 nucleotides of the SCN11Asv1 or SCN11Asv2 sequence that correspond to the respective novel exon junction or novel polynucleotide regions. In particular, for detection of SCN11Asv1, the probe comprises at least 20 nucleotides of the SCN11Asv1 sequence that corresponds to an exon junction polynucleotide created by the alternative splicing of exon 15 to exon 17 of the primary transcript of the SCN11A gene (see FIGS. 1A and 1B). For example, the polynucleotide sequence: 5′ TGAACAACAGAAGTCTGATG 3′ [SEQ ID NO 9] represents one embodiment of such an inventive SCN11Asv1 polynucleotide wherein a first 10 nucleotide region is complementary and hybridizable to the 3′ end of exon 15 of the SCN11A gene and a second 10 nucleotide region is complementary and hybridizable to the 5′ end of exon 17 of the SCN11A gene (see FIG. 1B).

In another embodiment, for detection of SCN11Asv2, the probe comprises at least 20 nucleotides of the SCN11Asv2 sequence that corresponds to an exon junction polynucleotide created by the alternative splicing of exon 6 to intron 6A of the primary transcript of the SCN11A gene (see FIGS. 1A and 1B). For example, the polynucleotide sequence: 5′ GAAGCTTATGA GTCTCTTAG 3′ [SEQ ID NO 10] represents one embodiment of such an inventive SCN11Asv2 polynucleotide wherein a first 10 nucleotide region is complementary and hybridizable to the 3′ end of exon 6 of the SCN11A gene and a second 10 nucleotide region is complementary and hybridizable to the 5′ end of intron 6A of the SCN11A gene (see FIG. 1B).

In another example, the polynucleotide sequence: 5′ TATGGAGAAGACCATT GCTT 3′ [SEQ ID NO 11] represents one embodiment of such an inventive SCN11Asv2 polynucleotide wherein a first 10 nucleotides region is complementary and hybridizable to the 3′ end of intron 6A of the SCN11A gene and a second 10 nucleotide region is complementary and hybridizable to the 5′ end of exon 7 of the SCN11A gene (see FIG. 1B).

In some embodiments, the first 20 nucleotides of a SCN11Asv1 probe comprise a first continuous region of 5 to 15 nucleotides that is complementary and hybridizable to the 3′ end of exon 15 and a second continuous region of 5 to 15 nucleotides that is complementary and hybridizable to the 5′ end of exon 17. In some embodiments, the first 20 nucleotides of a SCN11Asv2 probe comprise a first continuous region of 5 to 15 nucleotides that is complementary and hybridizable to the 5′ end of exon 6 and a second continuous region of 5 to 15 nucleotides that is complementary and hybridizable to the 5′ end of intron 6A of the SCN11A gene, or alternatively, the first 20 nucleotides of a SCN11Asv2 probe comprise a first continous region of 5 to 15 nucleotides that is complementary and hybridizable to the 3′ end of intron 6A and a second continuous region of 5 to 15 nucleotides that is complementary and hybridizable to the 5′ end of exon 7.

In other embodiments, the SCN11Asv1 or SCN11Asv2 polynucleotide comprises at least 40, 60, 80 or 100 nucleotides of the SCN11Asv1 or SCN11Asv2 sequence, respectively, that correspond to a junction polynucleotide region created by the alternative splicing of exon 15 to exon 17 in the case of SCN11Asv1, or in the case of SCN11Asv2, the lack of splicing of exon 6 to exon 7 resulting in the retention of intron 6A of the primary transcript of the SCN11A gene. In embodiments involving SCN11Asv1, the SCN11Asv1 polynucleotide is selected to comprise a first continuous region of at least 5 to 15 nucleotides that is complementary and hybridizable to the 3′ end of exon 15 and a second continuous region of at least 5 to 15 nucleotides that is complementary and hybridizable to the 5′ end of exon 17. Similarly, in embodiments involving SCN11Asv2, the SCN11Asv2 polynucleotide is selected to comprise a first continuous region of at least 5 to 15 nucleotides that is complementary and hybridizable to the 3′ end of exon 6 and a second continuous region of at least 5 to 15 nucleotides that is complementary and hybridizable to the 5′ end of intron 6A, or the SCN11Asv2 polynucleotide is selected to comprise a first continuous region of at least 5 to 15 nucleotides that is complementary and hybridizable to the 3′ end of intron 6A and a second continuous region of at least 5 to 15 nucleotides that is complementary and hybridizable to the 5′ end of exon 7 of the SCN11A gene. As will be apparent to a person of skill in the art, a large number of different polynucleotide sequences from the region of the exon 15 to exon 17 splice junction, the exon 6 to intron 6A, or intron 6A to exon 7 splice junction may be selected which will, under appropriate hybridization conditions, have the capacity to detectably hybridize to SCN11Asv1 or SCN11Asv2 polynucleotides and yet will hybridize to a much less extent or not at all to SCN11A isoform polynucleotides wherein exon 15 is not spliced to exon 17, wherein exon 6 is not spliced to intron 6A or wherein intron 6A is not spliced to exon 7.

Preferably, non-complementary nucleic acid that is present has a particular purpose such as being a reporter sequence or being a capture sequence. However, additional nucleic acid need not have a particular purpose as long as the additional nucleic acid does not prevent the SCN11Asv1 or SCN11Asv2 nucleic acid from distinguishing between target polynucleotides, e.g., SCN11Asv1 or SCN11Asv2 polynucleotides, and non-target polynucleotides, including, but not limited to SCN11A polynucleotides not comprising the exon 15 to exon 17 splice junction, or exon 6 to intron 6A or intron 6A to exon 7 splice junctions found in SCN11Asv1 or SCN11Asv2, respectively.

Hybridization occurs through complementary nucleotide bases. Hybridization conditions determine whether two molecules, or regions, have sufficiently strong interactions with each other to form a stable hybrid.

The degree of interaction between two molecules that hybridize together is reflected by the melting temperature (Tₘ) of the produced hybrid. The higher the Tₘ the stronger the interactions and the more stable the hybrid. Tₘ is effected by different factors well known in the art such as the degree of complementarity, the type of complementary bases present (e.g., A-T hybridization versus G-C hybridization), the presence of modified nucleic acid, and solution components (e.g., Sambrook, et al., in Molecular Cloning, A Laboratory Manual, 2^{nd} Edition, Cold Spring Harbor Laboratory Press, 1989).

Stable hybrids are formed when the Tₘ of a hybrid is greater than the temperature employed under a particular set of hybridization assay conditions. The degree of specificity of a probe can be varied by adjusting the hybridization stringency conditions. Detecting probe hybridization is facilitated through the use of a detectable label. Examples of detectable labels include luminescent, enzymatic, and radioactive labels.

Examples of stringency conditions are provided in Sambrook, et al., in Molecular Cloning, A Laboratory Manual, 2^{nd} Edition, Cold Spring Harbor Laboratory Press, 1989. An example of high stringency conditions is as follows: Prehybridization of filters containing DNA is carried out for 2 hours to overnight at 65° C. in buffer composed of 6×SSC, 5× Denhardt's solution, and 100 μg/ml denatured salmon sperm DNA. Filters are hybridized for 12 to 48 hours at 65° C. in prehybridization mixture containing 100 μg/ml denatured salmon sperm DNA and 5-20×10⁶ cpm of ³²P-labeled probe. Filter washing is done at 37° C. for 1 hour in a solution containing 2×SSC, 0.1% SDS. This is followed by a wash in 0.1× SSC, 0.1% SDS at 50° C. for 45 minutes before autoradiography. Other procedures using conditions of high stringency would include, for example, either a hybridization step carried out in 5×SSC, 5× Denhardt's solution, 50% formamide at 42° C. for 12 to 48 hours or a washing step carried out in 0.2×SSPE, 0.2% SDS at 65° C. for 30 to 60 minutes.

Recombinant Expression

SCN11Asv1 or SCN11Asv2 polynucleotides, such as those comprising SEQ ID NO 5 or SEQ ID NO 7, respectively, can be used to make SCN11Asv1 or SCN11Asv2 polypeptides, respectively. In particular, SCN11Asv1 or SCN11Asv2 polypeptides can be expressed from recombinant nucleic acids in a suitable host or in vitro using a translation system. Recombinantly expressed SCN11Asv1 or SCN11Asv2 polypeptides can be used, for example, in assays to screen for compounds that bind SCN11Asv1 or SCN11Asv2, respectively. Alternatively, SCN11Asv1 or SCN11Asv2 polypeptides can also be used to screen for compounds that bind to one or more SCN11A or sodium channel isoforms, but do not bind to SCN11Asv1 or SCN11Asv2, respectively.

In some embodiments, expression is achieved in a host cell using an expression vector. An expression vector contains recombinant nucleic acid encoding a polypeptide along with regulatory elements for proper transcription and processing. The regulatory elements that may be present include those naturally associated with the recombinant nucleic acid and exogenous regulatory elements not naturally associated with the recombinant nucleic acid. Exogenous regulatory elements such as an exogenous promoter can be useful for expressing recombinant nucleic acid in a particular host.

Generally, the regulatory elements that are present in an expression vector include a transcriptional promoter, a ribosome binding site, a terminator, and an optionally present operator. Another preferred element is a polyadenylation signal providing for processing in eukaryotic cells. Preferably, an expression vector also contains an origin of replication for autonomous replication in a host cell, a selectable marker, a limited number of useful restriction enzyme sites, and a potential for high copy number. Examples of expression vectors are cloning vectors, modified cloning vectors, and specifically designed plasmids and viruses.

Expression vectors providing suitable levels of polypeptide expression in different hosts are well known in the art. Mammalian expression vectors well known in the art include, but are not restricted to, pcDNA3 (Invitrogen, Carlsbad Calif.), pSecTag2 (Invitrogen), pMC1neo (Stratagene, La Jolla Calif.), pXT1 (Stratagene), pSG5 (Stratagene), pCMVLacl (Stratagene), pCI-neo (Promega), EBO-pSV2-neo (ATCC 37593), pBPV-1(8-2) (ATCC 37110), pdBPV-MMTneo(342-12) (ATCC 37224), pRSVgpt (ATCC 37199), pRSVneo (ATCC 37198), pSV2-dhfr (ATCC 37146) and pUCTag (ATCC 37460). Bacterial expression vectors well known in the art include pET11a (Novagen), pBluescript SK (Stratagene, La Jolla), pQE-9 (Qiagen Inc., Valencia), lambda gt11 (Invitrogen), pcDNAII (Invitrogen), and pKK223-3 (Pharmacia). Fungal cell expression vectors well known in the art include pRS416 (ATCC 87521), pPICZ (Invitrogen), pYES2 (Invitrogen), and Pichia expression vector (Invitrogen). Insect cell expression vectors well known in the art include Blue Bac III (Invitrogen), pBacPAK8 (CLONTECH, Inc., Palo Alto) and PfastBacHT (Invitrogen, Carlsbad, Calif.).

Recombinant host cells may be prokaryotic or eukaryotic. Examples of recombinant host cells include the following: bacteria such as E. coli; fungal cells such as yeast; mammalian cells such as human, bovine, porcine, monkey and rodent; and insect cells such as Drosophila and silkworm derived cell lines. Commercially available mammalian cell lines include L cells L-M(TK⁻) (ATCC CCL 1.3), L cells L-M (ATCC CCL 1.2), 293 (ATCC CRL 1573), Raji (ATCC CCL 86), CV-1 (ATCC CCL 70), COS-1 (ATCC CRL 1650), COS-7 (ATCC CRL 1651), CHO-K1 (ATCC CCL 61), 3T3 (ATCC CCL 92), NIH/3T3 (ATCC CRL 1658), HeLa (ATCC CCL 2), C127I (ATCC CRL 1616), BS-C-1 (ATCC CCL 26) MRC-5 (ATCC CCL 171), and HEK 293 cells (ATCC CRL-1573).

Recombinant DNA molecules that feature precise fusions of polynucleotide sequences can also be assembled using standard recombinational subcloning techniques. Recombination-mediated, PCR-directed, or PCR-independent plasmid construction in yeast is well known in the art (see Hua et al., 1997, Plasmid 38:91-96; Hudson et al., 1997, Genome Res. 7(12):1169-1173; Oldenburg et al., 1997, Nucleic Acids Res. 25(2):451-452; Raymond et al., 1999, BioTechniques 26(1)134-8, 140-1). Overlapping sequences between the donor DNA fragments and the acceptor plasmid permit recombination in yeast. An example of recombination-mediated plasmid construction in Saccharomyces cerevisiae is described in Oldenburg et al., 1997, Nucleic Acids Res. 25(2):451-452: a DNA segment of interest was amplified by PCR so that the PCR product had 20-40 bp of homology at each end to the region of the plasmid at which recombination was to occur. The PCR product and linearized plasmid were co-transformed into yeast, and recombination resulted in replacement of the region between the homologous sequences on the plasmid with the region carried by the PCR fragment. The recombinational method of plasmid construction bypasses the need for extensive modification and ligation steps and does not rely on available restriction sites. These cloning vectors can then be utilized for protein expression in multiple systems.

To enhance expression in a particular host it may be useful to modify the sequence provided in SEQ ID NO 5 or SEQ ID NO 7 to take into account codon usage of the host. Codon usages of different organisms are well known in the art (see, Ausubel, Current Protocols in Molecular Biology, John Wiley, 1987-1998, Supplement 33 Appendix IC).

Expression vectors may be introduced into host cells using standard techniques. Examples of such techniques include transformation, transfection, lipofection, protoplast fusion, and electroporation.

Nucleic acids encoding for a polypeptide can be expressed in a cell without the use of an expression vector employing, for example, synthetic mRNA or native mRNA. Additionally, mRNA can be translated in various cell-free systems such as wheat germ extracts and reticulocyte extracts, as well as in cell based systems, such as frog oocytes. Introduction of mRNA into cell based systems can be achieved, for example, by microinjection or electroporation.

SCN11Asv1 and SCN11Asv2 Polypeptides

SCN11Asv1 polypeptides contain an amino acid sequence comprising, consisting or consisting essentially of SEQ ID NO 6. SCN11Asv2 polypeptides contain an amino acid sequence comprising, consisting, or consisting essentially of SEQ ID NO 8. SCN11Asv1 [SEQ ID NO 6] has 97.9% amino acid sequence identity as compared to the SCN11A reference protein (AAF17480). SCN11Asv2 [SEQ ID NO 8] has 98.1% amino acid sequence identity as compared to the SCN11A reference protein (AAF17480). SCN11Asv1 or SCN11Asv2 polypeptides have a variety of uses, such as providing a marker for the presence of SCN11Asv1 or SCN11Asv2, respectively; use as an immunogen to produce antibodies binding to SCN11Asv1 or SCN11Asv2, respectively; use as a target to identify compounds binding selectively to SCN11Asv1 or SCN11Asv2, respectively; or use in an assay to identify compounds that bind to one or more SCN11A or sodium channel isoforms but do not bind to or interact with SCN11Asv1 or SCN11Asv2, respectively.

In chimeric polypeptides containing one or more regions from SCN11Asv1 or SCN11Asv2 and one or more regions not from SCN11Asv1 or SCN11Asv2, respectively, the region(s) not from SCN11Asv1 or SCN11Asv2 can be used, for example, to achieve a particular purpose or to produce a polypeptide that can substitute for SCN11Asv1, or SCN11Asv2, or fragments thereof. Particular purposes that can be achieved using chimeric SCN11Asv1 or SCN11Asv2 polypeptides include providing a marker for SCN11Asv1 or SCN11Asv2 activity, respectively and altering the activity and regulation of the SCN11A sodium channel.

Polypeptides can be produced using standard techniques including those involving chemical synthesis and those involving biochemical synthesis. Techniques for chemical synthesis of polypeptides are well known in the art (see e.g., Vincent, in Peptide and Protein Drug Delivery, New York, N.Y., Dekker, 1990).

Biochemical synthesis techniques for polypeptides are also well known in the art. Such techniques employ a nucleic acid template for polypeptide synthesis. The genetic code providing the sequences of nucleic acid triplets coding for particular amino acids is well known in the art (see, e.g., Lewin GENES IV, p. 119, Oxford University Press, 1990). Examples of techniques for introducing nucleic acid into a cell and expressing the nucleic acid to produce protein are provided in references such as Ausubel, Current Protocols in Molecular Biology, John Wiley, 1987-1998, and Sambrook, et al., in Molecular Cloning, A Laboratory Manual, 2^{nd} Edition, Cold Spring Harbor Laboratory Press, 1989.

Functional SCN11Asv1 and SCN11Asv2

Functional SCN11Asv1 and SCN11Asv2 are different protein isoforms of SCN11A. The identification of the amino acid and nucleic acid sequences of SCN11Asv1 or SCN11Asv2 provides tools for obtaining functional proteins related to SCN11Asv1 or SCN11Asv2, respectively, from other sources, for producing SCN11Asv1 or SCN11Asv2 chimeric proteins, and for producing functional derivatives of SEQ ID NO 6 or SEQ ID NO 8.

SCN11Asv1 or SCN11Asv2 polypeptides can be readily identified and obtained based on their sequence similarity to SCN11Asv1 [SEQ ID NO 6] or SCN11Asv2 [SEQ ID NO 8], respectively. In particular, SCN11Asv1 lacks the amino acids encoded by exon 16 of the SCN11A gene. The deletion of exon 16 and the splicing of exon 15 to exon 17 of the SCN11A heteronuclear RNA (hnRNA) transcript do not alter the protein reading frame at the exon 15 to exon 17 splice junction. Thus, the SCN11Asv1 polypeptide is lacking the amino acids encoded by the nucleotides corresponding to exon 16 of the SCN11A hnRNA transcript as compared to the SCN11A reference sequence. The SCN11Asv2 polypeptides contain additional amino acids, encoded by nucleotides located after the splice junction that results from the retention of intron 6A [SEQ ID NO 4] of the SCN11A gene. The addition of intron 6A and the splicing of exon 6 to intron 6A, and intron 6A to exon 7 of the SCN11A hnRNA transcript do not alter the protein reading frame at the exon 6 to intron 6A and intron 6A to exon 7 splice junctions. Thus, the SCN11Asv2 polypeptide contains 34 additional amino acids encoded by nucleotides corresponding to intron 6A [SEQ ID NO 4] of the SCN11A hnRNA as compared to the SCN11A reference sequence.

Both the amino acid and nucleic acid sequences of SCN11Asv1 or SCN11Asv2 can be used to help identify and obtain SCN11Asv1 or SCN11Asv2 polypeptides, respectively. For example, SEQ ID NO 5 can be used to produce degenerative nucleic acid probes or primers for identifying and cloning nucleic acid polynucleotides encoding for a SCN11Asv1 polypeptide. In addition, polynucleotides comprising, consisting, or consisting essentially of SEQ ID NO 5 or fragments thereof, can be used under conditions of moderate stringency to identify and clone nucleic acids encoding SCN11Asv1 polypeptides from a variety of different organisms. The same methods can also be performed with polynucleotides comprising, consisting, or consisting essentially of SEQ ID NO 7, or fragments thereof, to identify and clone nucleic acids encoding SCN11Asv2.

The use of degenerative probes and moderate stringency conditions for cloning is well known in the art. Examples of such techniques are described by Ausubel, Current Protocols in Molecular Biology, John Wiley, 1987-1998, and Sambrook, et al., in Molecular Cloning, A Laboratory Manual, 2^{nd} Edition, Cold Spring Harbor Laboratory Press, 1989.

Starting with SCN11Asv1 or SCN11Asv2 obtained from a particular source, derivatives can be produced. Such derivatives include polypeptides with amino acid substitutions, additions and deletions. Changes to SCN11Asv1 or SCN11Asv2 to produce a derivative having essentially the same properties should be made in a manner not altering the tertiary structure of SCN11Asv1 or SCN11Asv2, respectively.

Differences in naturally occurring amino acids are due to different R groups. An R group affects different properties of the amino acid such as physical size, charge, and hydrophobicity. Amino acids are can be divided into different groups as follows: neutral and hydrophobic (alanine, valine, leucine, isoleucine, proline, tryptophan, phenylalanine, and methionine); neutral and polar (glycine, serine, threonine, tryosine, cysteine, asparagine, and glutamine); basic (lysine, arginine, and histidine); and acidic (aspartic acid and glutamic acid).

Generally, in substituting different anino acids it is preferable to exchange amino acids having similar properties. Substituting different amino acids within a particular group, such as substituting valine for leucine, arginine for lysine, and asparagine for glutamine are good candidates for not causing a change in polypeptide functioning.

Changes outside of different amino acid groups can also be made. Preferably, such changes are made taking into account the position of the amino acid to be substituted in the polypeptide. For example, arginine can substitute more freely for nonpolar amino acids in the interior of a polypeptide then glutamate because of its long aliphatic side chain (See, Ausubel, Current Protocols in Molecular Biology, John Wiley, 1987-1998, Supplement 33 Appendix IC).

SCN11Asv1 and SCN11Asv2 Antibodies

Antibodies recognizing SCN11Asv1 or SCN11Asv2 can be produced using a polypeptide containing SEQ ID NO 6 in the case of SCN11Asv1 or SEQ ID NO 8 in the case of SCN11Asv2, respectively, or a fragment thereof as an immunogen. Preferably, a SCN11Asv1 polypeptide used as an immunogen consists of a polypeptide of SEQ ID NO 6 or a SEQ ID NO 6 fragment having at least 10 contiguous amino acids in length corresponding to the polynucleotide region representing the junction resulting from the splicing of exon 15 to exon 17 of the SCN11A gene. When a SCN11Asv2 polypeptide is used as an immunogen, preferably it consists of a polypeptide derived from SEQ ID NO 8 or a SEQ ID NO 8 fragment, having at least 10 contiguous amino acids in length corresponding to a polynucleotide region representing the junction from exon 6 to intron 6A of the SCN11A gene. Alternatively, when a SCN11Asv2 polypeptide is used as an immunogen, preferably it consists of a polypeptide derived from SEQ ID NO 8 or a SEQ ID NO 8 fragment, having at least 10 contiguous amino acids in length corresponding to a polynucleotide region representing the junction from intron 6A to exon 7 of the SCN11A gene.

In some embodiments where, for example, SCN11Asv1 polypeptides are used to develop antibodies that bind specifically to SCN11Asv1 and not to other isoforms of SCN11A, the SCN11Asv1 polypeptides comprise at least 10 amino acids of the SCN11Asv1 polypeptide sequence corresponding to a junction polynucleotide region created by the alternative splicing of exon 15 to exon 17 of the primary transcript of the SCN11A gene (see FIG. 1). For example, the amino acid sequence: amino terminus-EPEQQKSDVT-carboxy terminus [SEQ ID NO 12] represents one embodiment of such an inventive SCN11Asv1 polypeptide wherein a first 5 amino acid region is encoded by a nucleotide sequence at the 3′ end of exon 15 of the SCN11A gene and a second 5 amino acid region is encoded by the nucleotide sequence directly after the novel splice junction. Preferably, at least 10 amino acids of the SCN11Asv1 polypeptide comprise a first continuous region of 2 to 8 amino acids that is encoded by nucleotides at the 3′ end of exon 15 and a second continuous region of 2 to 8 amino acids that is encoded by nucleotides at the 5′ end of exon 17.

In other embodiments where, for example, SCN11Asv2 polypeptides are used to develop antibodies that bind specifically to SCN11Asv2 and not to other SCN11A isoforms, the SCN11Asv2 polypeptides comprise at least 10 amino acids of the SCN11Asv2 polypeptide sequences corresponding to a junction polynucleotide region created by the retention of intron 6A of the primary transcript of the SCN11A gene (see FIG. 1). For example, the amino acid sequence: amino terminus-NPEAYESLSS-carboxy terminus [SEQ ID NO 13], represents one embodiment of such an inventive SCN11Asv2 polypeptide wherein a first 5 amino acid region is coded by a nucleotide sequence at the 3′ end of exon 6 of the SCN11A gene and a second 5 amino acid region is coded by a nucleotide sequence at the 5′ end of intron 6A. Preferably, at least 10 amino acids of the SCN11Asv2 polypeptide comprises a first continuous region of 2 to 8 amino acids that is coded by nucleotides at the 3′ end of exon 6 and a second continuous region of 2 to 8 amino acids that is coded by nucleotides at the 5′ end intron 6A. Alternatively, in the case of SCN11Asv2 [SEQ ID NO 8], the amino acid sequence: amino terminus-ASYGEDHCFE-carboxy terminus [SEQ ID NO 14], represents one embodiment of such an inventive SCN11Asv2 polypeptide wherein a first 5 amino acid region is coded by a nucleotide sequence at the 3′ end of intron 6A of the SCN11A gene and a second 5 amino acid region is coded by a nucleotide sequence at the 5′ end of exon 7. Preferably, at least 10 amino acids of the SCN11Asv2 polypeptide comprises a first continuous region of 2 to 8 amino acids that is coded by nucleotides at the 3′ end of intron 6A and a second continuous region of 2 to 8 amino acids that is coded by nucleotides at the 5′ end exon 7.

In other embodiments, SCN11Asv1-specific antibodies are made using a SCN11Asv1 polypeptide that comprises at least 20, 30, 40 or 50 amino acids of the SCN11Asv1 sequence that corresponds to a junction polynucleotide region created by the alternative splicing of exon 15 to exon 17 of the primary transcript of the SCN11A gene. In each case the SCN11Asv1 polypeptides are selected to comprise a first continuous region of at least 5 to 15 amino acids that is encoded by nucleotides at the 3′ end of exon 15 and a second continuous region of 5 to 15 amino acids that is encoded by nucleotides directly after the novel splice junction.

In other embodiments, SCN11Asv2-specific antibodies are made using a SCN11Asv2 polypeptide that comprises at least 20, 30, 40 or 50 amino acids of the SCN11Asv2 sequence that corresponds to a junction polynucleotide region created by the retention of intron 6A [SEQ ID NO 4] of the primary transcript of the SCN11A gene. In one case the SCN11Asv2 polypeptides are selected to comprise a first continuous region of at least 5 to 15 amino acids that is coded by nucleotides at the 3′ end of exon 6 and a second continuous region of 5 to 15 amino acids that is encoded by nucleotides directly after the novel junction in intron 6A of the SCN11A gene. Alternatively, SCN11Asv2 polypeptides are selected to comprise a first continuous region of at least 5 to 15 amino acids that is coded by nucleotides at the 3′ end of intron 6A and a second continuous region of 5 to 15 amino acids that is encoded by nucleotides directly after the novel junction created by splicing of intron 6A to exon 7 of the SCN11A gene.

Antibodies to SCN11Asv1 or SCN11Asv2 have different uses, such as to identify the presence of SCN11Asv1 or SCN11Asv2, respectively, and to isolate SCN11Asv1 or SCN11Asv2 polypeptides, respectively. Identifying the presence of SCN11Asv1 can be used, for example, to identify cells producing SCN11Asv1. Such identification provides an additional source of SCN11Asv1 and can be used to distinguish cells known to produce SCN11Asv1 from cells that do not produce SCN11Asv1. For example, antibodies to SCN11Asv1 can distinguish human cells expressing SCN11Asv1 from human cells not expressing SCN11Asv1 or non-human cells (including bacteria) that do not express SCN11Asv1. Such SCN11Asv1 antibodies can also be used to determine the effectiveness of SCN11Asv1 ligands, using techniques well known in the art, to detect and quantify changes in the protein levels of SCN11Asv1 in cellular extracts, and in situ immunostaining of cells and tissues. In addition, the same above-described utilities also exist for SCN11Asv2-specific antibodies.

Techniques for producing and using antibodies are well known in the art. Examples of such techniques are described in Ausubel, Current Protocols in Molecular Biology, John Wiley, 1987-1998; Harlow, et al., Antibodies, A Laboratory Manual, Cold Spring Harbor Laboratory, 1988; and Kohler, et al., 1975 Nature 256:495-7.

SCN11Asv1 and SCN11Asv2 Binding Assay

A number of compounds known to modulate sodium channel activity have been disclosed including tetrodotoxin, saxitoxin, cadmium, and other neurotoxins such as α-scorpion toxin and sea anemone toxin (Akopian et al., 1996; Satin et al., 1992; Backz et al., 1992; reviewed in Catterall, W. A., 2000). Peptide sequences corresponding to the intracellular loop connecting Domains III and IV of the sodium channel α subunit have also been reported to have efficacy as pore blockers and can restore activation to sodium channels that are defective in inactivation (Eaholtz et al., 1994). The human SCN11A reference protein (AAF17480) has a serine in the SS2 segment of Domain I and is therefore expected to be resistant to tetrodotoxin. This is consistent with the finding that the rat SCN11A sodium channel expressed in HEK293T cells produced a tetrodotoxin resistant current (Tate et al, 1998, Nature Neurosci 1:653-655). Splice variants of the cockroach SCN8A sodium channel exhibit different gating properties and different sensitivities to deltamethrin, a pyrethroid insecticide (Tan et al., 2002, J. Neurosci. 22(13): 5300-5309), indicating that splice variant isoforms may have different sensitivities to ligands. Methods for expressing sodium channels in Xenopus oocytes and monitoring the activity of these channels, including analyzing the effect of compounds on the activity of sodium channel activity, have been described previously (Dietrich et al., 1998, J. Neurochem. 70(6): 2262-72; Keller et al., 2000, J. Pharmacol. Exp. Ther. 295(1): 367-72; Spampanato et al., 2001, J. Neurosci. 21(19): 7481-7490; Tan et al., 2002, J. Neurosci. 22(13): 5300-5309). Methods for screening compounds for their effects on sodium channel activity have also been disclosed (see for example US 2002/0025568; US 2002/0045159; WO 03/006103; Gonzales et al., 1999, Drug Discov. Today 4: 431-439). A person skilled in the art should be able to use these methods to screen SCN11Asv1 or SCN11Asv2 polypeptides for compounds that bind to, and in some cases functionally alter, each respective SCN11A isoform protein.

SCN11Asv1, SCN11Asv2 or fragments thereof, can be used in binding studies to identify compounds binding to or interacting with SCN11Asv1, SCN11Asv2, or fragments thereof, respectively. In one embodiment, SCN11Asv1, or a fragment thereof, can be used in binding studies with a sodium channel isoform protein, or a fragment thereof, to identify compounds that: bind to or interact with SCN11Asv1 and other sodium channel isoforms; bind to or interact with one or more other sodium channel isoforms and not with SCN11Asv1; bind to or interact with SCN11Asv1 and not with one or more other sodium channel isoforms. A similar series of compound screens can, of course, also be performed using SCN11Asv2 rather than, or in addition to, SCN11Asv1. Such binding studies can be performed using different formats including competitive and non-competitive formats. Further competition studies can be carried out using additional compounds determined to bind to SCN11Asv1, SCN11Asv2, other SCN11A isoforms, or other sodium channel isoforms.

The particular SCN11Asv1 or SCN11Asv2 sequence involved in ligand binding can be identified using labeled compounds that bind to the protein and different protein fragments. Different strategies can be employed to select fragments to be tested to narrow down the binding region. Examples of such strategies include testing consecutive fragments about 15 amino acids in length starting at the N-terminus, and testing longer length fragments. If longer length fragments are tested, a fragment binding to a compound can be subdivided to further locate the binding region. Fragments used for binding studies can be generated using recombinant nucleic acid techniques.

In some embodiments, binding studies are performed using SCN11Asv1 expressed from a recombinant nucleic acid. Alternatively, recombinantly expressed SCN11Asv1 consists of the SEQ ID NO 6 amino acid sequence. In addition, binding studies are performed using SCN11Asv2 expressed from a recombinant nucleic acid. Alternatively, recombinantly expressed SCN11Asv2 consists of the SEQ ID NO 8 amino acid sequence.

Binding assays can be performed using individual compounds or preparations containing different numbers of compounds. A preparation containing different numbers of compounds having the ability to bind to SCN11Asv1 or SCN11Asv2 can be divided into smaller groups of compounds that can be tested to identify the compound(s) binding to SCN11Asv1.

Binding assays can be performed using recombinantly produced SCN11Asv1 or SCN11Asv2 present in different environments. Such environments include, for example, cell extracts and purified cell extracts containing a SCN11Asv1 or SCN11Asv2 recombinant nucleic acid; and also include, for example, the use of a purified SCN11Asv1 or SCN11Asv2 polypeptide produced by recombinant means which is introduced into different environments.

In one embodiment of the invention, a binding method is provided for screening for a compound able to bind selectively to SCN11Asv1. The method comprises the steps: providing a SCN11Asv1 polypeptide comprising SEQ ID NO 6; providing a sodium channel isoform polypeptide that is not SCN11Asv1; contacting the SCN11Asv1 polypeptide and the sodium channel isoform polypeptide that is not SCN11Asv1 with a test preparation comprising one or more test compounds; and then determining the binding of the test preparation to the SCN11Asv1 polypeptide and to the sodium channel isoform polypeptide that is not SCN11Asv1, wherein a test preparation that binds to the SCN11Asv1 polypeptide, but does not bind to the sodium channel isoform polypeptide that is not SCN11Asv1, contains one or more compounds that selectively bind to SCN11Asv1.

In one embodiment of the invention, a binding method is provided for screening for a compound able to bind selectively to SCN11Asv2. The method comprises the steps: providing a SCN11Asv2 polypeptide comprising SEQ ID NO 8; providing a sodium channel isoform polypeptide that is not SCN11Asv2; contacting the SCN11Asv2 polypeptide and the sodium channel isoform polypeptide that is not SCN11Asv2 with a test preparation comprising one or more test compounds; and then determining the binding of the test preparation to the SCN11Asv2 polypeptide and to the sodium channel isoform polypeptide that is not SCN11Asv2, wherein a test preparation that binds to the SCN11Asv2 polypeptide, but does not bind to the sodium channel isoform polypeptide that is not SCN11Asv2, contains one or more compounds that selectively bind to SCN11Asv2.

In another embodiment of the invention, a binding method is provided for screening for a compound able to bind selectively to a sodium channel isoform polypeptide that is not SCN11Asv1. The method comprises the steps: providing a SCN11Asv1 polypeptide comprising SEQ ID NO 6; providing a sodium channel isoform polypeptide that is not SCN11Asv1; contacting the SCN11Asv1 polypeptide and the sodium channel isoform polypeptide that is not SCN11Asv1 with a test preparation comprising one or more test compounds; and then determining the binding of the test preparation to the SCN11Asv1 polypeptide and the sodium channel isoform polypeptide that is not SCN11Asv1, wherein a test preparation that binds the sodium channel isoform polypeptide that is not SCN11Asv1, but does not bind SCN11Asv1, contains a compound that selectively binds the sodium channel isoform polypeptide that is not SCN11Asv1. Alternatively, the above method can be used to identify compounds that bind selectively to a sodium channel polypeptide that is not SCN11Asv2 by performing the method with SCN11Asv2 protein comprising SEQ ID NO 8.

The above-described selective binding assays can also be performed with a polypeptide fragment of SCN11Asv1 or SCN11Asv2, wherein the polypeptide fragment comprises at least 10 consecutive amino acids that are coded by a nucleotide sequence that bridges the junction created by the splicing of the 3′ end of exon 15 to the 5′ end of exon 17 in the case of SCN11Asv2, by the splicing of the 3′ end of exon 6 to the 5′ end of intron 6A or by the splicing of intron 6A to exon 7 in the case of SCN11Asv2. Similarly, the selective binding assays may also be performed using a polypeptide fragment of a sodium channel isoform polypeptide that is not SCN11Asv1 or SCN11Asv2, wherein the polypeptide fragment comprises at least 10 consecutive amino acids that are coded by: a) a nucleotide sequence that is contained within exon 16 of the SCN11A gene; b) a nucleotide sequence that is contained within intron 6A [SEQ ID NO 4] of the SCN11A gene or c) a nucleotide sequence that bridges the junction created by the splicing of the 3′ end of exon 15 to the 5′ end of exon 16 of the SCN11A gene, or the splicing of the 3′ end of exon 6 to the 5′ end of exon 7 of the SCN11A gene.

SCN11A Functional Assays

SCN11A encodes the alpha subunit of a highly conserved voltage gated sodium channel that is implicated in neurological disorders such as chronic and acute pain. Splice variants of sodium channels may exhibit different voltage gate activity and different binding affinities for compounds, peptides and other small molecules. The identification of SCN11Asv1 and SCN11Asv2 as splice variants of SCN11A provides a means of screening for compounds that bind to SCN11Asv1 and/or SCN11Asv2 protein thereby altering the activity or regulation of SCN11Asv1 and/or SCN11Asv2 sodium channels. Assays involving a functional SCN11Asv1 or SCN11Asv2 polypeptide can be employed for different purposes, such as selecting for compounds active at SCN11Asv1 or SCN11Asv2; evaluating the ability of a compound to affect the ion channel activity of each respective splice variant; and mapping the activity of different SCN11Asv1 and SCN11Asv2 regions. SCN11Asv1 and SCN11Asv2 activity can be measured using different techniques such as: detecting a change in the intracellular conformation of SCN11Asv1 or SCN11Asv2; detecting a change in the intracellular location of SCN11Asv1 or SCN11Asv2; or measuring the ion channel activity of SCN11Asv1 or SCN11Asv2.

Recombinantly expressed SCN11Asv1 and SCN11Asv2 can be used to facilitate the determination of whether a compound is active at SCN11Asv1 and SCN11Asv2. For example, SCN11Asv1 and SCN11Asv2 can be expressed by an expression vector in a cell line and used in a co-culture growth assay, such as described in WO 99/59037, to identify compounds that bind to SCN11Asv1 and SCN11Asv2. For example, SCN11Asv1 can be expressed by an expression vector in a human kidney cell line 293 and used in a co-culture growth assay, such as described in U.S. patent application Ser. No. 20020061860, to identify compounds that bind to SCN11Asv1 or SCN11ASv2.

Techniques for measuring voltage gated ion channel activity are well known in the art. Methods for expressing sodium channels in Xenopus oocytes and monitoring the activity of these channels, including analyzing the effect of compounds on the activity of sodium channel activity, have been described previously (Dietrich et al., 1998, J. Neurochem. 70(6): 2262-72; Keller et al., 2000, J. Pharmacol. Exp. Ther. 295(1): 367-72; Spampanato et al., 2001, J. Neurosci. 21(19): 7481-7490; Tan et al., 2002, J. Neurosci. 22(13): 5300-5309). The patch clamp technique measures ion current through ion channel proteins and can be used to analyze the effect of drugs on ion channel function. The activity of these channels can be measured electrically (single channel recording) or, alternatively, the patch can be ruptured allowing measurements of the combined channel activity of the entire cell membrane (whole cell recording) (Neher et al., 1978, Pflugers Arch. 375(2): 219-28; Sakman et al., 1984, Annu Rev Physiol. 46: 455-72; Neher et al., 1992, Sci. Am. 266(3): 44-51). Other methods for measuring ion channel activity include optical reading of voltage-sensitive dyes (Cohen et al., 1978, Annual Reviews of Neuroscience 1: 171-82) and extracellular recording of fast events using metal (Thomas et al., 1972, Exp. Cell Res. 74: 61-66) or field effect transistor (Fromherz et al., 1991, Science 252: 1290-1293) electrodes. High throughput methods for assaying ion channel activity have also been described (see WO 03/006103A2 and US 2002/0028480). A variety of other assays has been used to investigate the properties of sodium channels and therefore would also be applicable to the measurement of SCN11Asv1.1, SCN11Asv1.2, or SCN11Asv2 function.

SCN11Asv1 or SCN11Asv2 functional assays can be performed using cells expressing SCN11Asv1 or SCN11Asv2 at a high level. These proteins will be contacted with individual compounds or preparations containing different compounds. A preparation containing different compounds where one or more compounds affect SCN11Asv1 or SCN11Asv2 in cells over-producing SCN11Asv1 or SCN11Asv2 as compared to control cells containing an expression vector lacking SCN11Asv1 or SCN11Asv2 coding sequences, can be divided into smaller groups of compounds to identify the compound(s) affecting SCN11Asv1 or SCN11Asv2 activity, respectively.

SCN11Asv1 or SCN11Asv2 functional assays can be performed using recombinantly produced SCN11Asv1 or SCN11Asv2present in different environments. Such environments include, for example, cell extracts and purified cell extracts containing the SCN11Asv1 or SCN11Asv2 expressed from recombinant nucleic acid; and the use of purified SCN11Asv1 or SCN11Asv2 produced by recombinant means that is introduced into a different environment suitable for measuring ion channel activity.

### MODULATING SCN11Asv1 and SCN11Asv2 EXPRESSION

SCN11Asv1 or SCN11Asv2 expression can be modulated as a means for increasing or decreasing SCN11Asv1 SCN11Asv2 activity, respectively. Such modulation includes inhibiting the activity of nucleic acids encoding the SCN11A isoform target to reduce SCN11A isoform protein or polypeptide expression, or supplying SCN11A nucleic acids to increase the level of expression of the SCN11A target polypeptide thereby increasing SCN11A activity.

Inhibition of SCN11Asv1 and SCN11Asv2 Activity

SCN11Asv1 or SCN11Asv2 nucleic acid activity can be inhibited using nucleic acids recognizing SCN11Asv1 or SCN11Asv2 nucleic acid and affecting the ability of such nucleic acid to be transcribed or translated. Inhibition of SCN11Asv1 or SCN11Asv2 nucleic acid activity can be used, for example, in target validation studies.

A preferred target for inhibiting SCN11Asv1 or SCN11Asv2 is mRNA stability and translation. The ability of SCN11Asv1 or SCN11Asv2 mRNA to be translated into a protein can be effected by compounds such as anti-sense nucleic acid, RNA interference (RNAi) and enzymatic nucleic acid.

Anti-sense nucleic acid can hybridize to a region of a target mRNA. Depending on the structure of the anti-sense nucleic acid, anti-sense activity can be brought about by different mechanisms such as blocking the initiation of translation, preventing processing of mRNA, hybrid arrest, and degradation of mRNA by RNAse H activity.

RNA inhibition (RNAi) using shRNA or siRNA molecules can also be used to prevent protein expression of a target transcript. This method is based on the interfering properties of double-stranded RNA derived from the coding region of a gene that disrupts the synthesis of protein from transcribed RNA.

Enzymatic nucleic acids can recognize and cleave other nucleic acid molecules. Preferred enzymatic nucleic acids are ribozymes.

General structures for anti-sense nucleic acids, RNAi and ribozymes, and methods of delivering such molecules, are well known in the art. Methods for using RNAi to modify sodium channel activity have been described previously (Keller et al., 2000, J. Pharmacol. Exp. Ther. 295(1): 367-72). Modified and unmodified nucleic acids can be used as anti-sense molecules, RNAi and ribozymes. Different types of modifications can affect certain RNA activities such as the ability to be cleaved by RNAse H, and can affect nucleic acid stability. Examples of references describing different anti-sense molecules, and ribozymes, and the use of such molecules, are provided in U.S. Pat. Nos. 5,849,902; 5,859,221; 5,852,188; and 5,616,459. Examples of organisms in which RNAi has been used to inhibit expression of a target gene include: C. elegans (Tabara, et al., 1999, Cell 99, 123-32; Fire, et al., 1998, Nature 391, 806-11), plants (Hamilton and Baulcombe, 1999, Science 286, 950-52), Drosophila (Hammond, et al., 2001, Science 293, 1146-50; Misquitta and Patterson, 1999, Proc. Nat. Acad. Sci. 96, 1451-56; Kennerdell and Carthew, 1998, Cell 95, 1017-26), and mammalian cells (Bernstein, et al., 2001, Nature 409, 363-6; Elbashir, et al., 2001, Nature 411, 494-8).

Increasing SCN11Asv1 and SCN11Asv2 Expression

Nucleic acids encoding for SCN11Asv1 or SCN11Asv2 can be used, for example, to cause an increase in SCN11A activity or to create a test system (e.g., a transgenic animal) for screening for compounds affecting SCN11Asv1 or SCN11Asv2 expression, respectively. Nucleic acids can be introduced and expressed in cells present in different environments.

Guidelines for pharmaceutical administration in general are provided in, for example, Remington's Pharmaceutical Sciences, 18^{th} Edition, supra, and Modern Pharmaceutics, 2^{nd} Edition, supra Nucleic acid can be introduced into cells present in different environments using in vitro, in vivo, or ex vivo techniques. Examples of techniques useful in gene therapy are illustrated in Gene Therapy & Molecular Biology: From Basic Mechanisms to Clinical Applications, Ed. Boulikas, Gene Therapy Press, 1998.

### EXAMPLES

Examples are provided below to further illustrate different features and advantages of the present invention. The examples also illustrate useful methodology for practicing the invention. These examples do not limit the claimed invention.

### Example 1

### Identification of SCN11 sv1 and SCN11Asv2 Using RT-PCR

Publicly available SCN11A mRNA transcripts varied from each other at one or more single nucleotide positions. The exon sequences of SCN11A mRNA (AF188679) exhibited a perfect match to the corresponding regions of the human genome sequence, so AF188679 and its protein product, AAF17480, were used as reference sequences. No evidence of alternative splicing of the SCN11A gene was observed in publicly available mRNA and EST databases. Alternatively spliced isoforms of TTX-resistant SCN5A sodium channel, resulting from an exon drop in the intracellular loop 2 between Domains II and III, were identified in mouse (Zimmer et al, 2002, Am. J. Physio. Heart Circ. Physiol. 282:H1007-H1017). The shorter of the SCN5A isoforms (mH1-3) did not express functional sodium channels. Alternatively spliced isoforms of human SCN11A were sought using information on the paralogous SCN5A.

The structure of SCN11A mRNA in the region corresponding to exons 15 to 19, which corresponds to loop 2 between Domains II and III of SCN11A, was determined for human dorsal root ganglia (DRG) using an RT-PCR based assay. Total RNA isolated from DRG was obtained from BD Biosciences Clontech (Palo Alto, Calif.). RT-PCR primers were selected that were complementary to sequences in exon 15 and exon 19 of the reference exon coding sequences in SCN11A (AF188679). Based upon the nucleotide sequence of SCN11A mRNA, the SCN11A exon 15 and exon 19 primer set (hereafter SCN11A₁₅₋₁₉ primer set) was expected to amplify a 574 base pair amplicon representing the “reference” SCN11A mRNA region. The SCN11A exon 15 forward primer has the sequence: 5′ GATGACGTTGAATTTTCTGGTGAAG ATA 3′ [SEQ ID NO 15]; and the SCN11A exon 19 reverse primer has the sequence: 5° CAAAT CCGAAGGCTACCCATTTTAGTA 3′ [SEQ ID NO 16].

Twenty-five ng of total RNA from DRG was subjected to a one-step reverse transcription-PCR amplification protocol using the Qiagen, Inc. (Valencia, Calif.), One-Step RT-PCR kit, using the following cycling conditions:
50° C. for 30 minutes;
95° C. for 15 minutes;
35 cycles of:
   94° C. for 30 seconds;
   63.5° C. for 40 seconds;
   72° C. for 50 seconds; then
   72° C. for 10 minutes.

RT-PCR amplification products (amplicons) were size fractionated on a 2% agarose gel. Selected amplicon fragments were manually extracted from the gel and purified with a Qiagen Gel Extraction Kit. Purified amplicon fragments were cloned into an Invitrogen pCR2.1 vector using the reagents and instructions provided with the TOPO TA cloning kit (Invitrogen, Carlsbad, Calif.). Clones were then sequenced from each end (using the same primers used for RT-PCR) by Qiagen Genomics, Inc. (Bothell, Wash.).

At least one different RT-PCR amplicon was obtained from human DRG RNA samples using the SCN11A₁₅₋₁₉ primer set (data not shown). The DRG sample assayed exhibited the expected amplicon size of 574 base pairs for normally spliced SCN11A mRNA. In addition, the DRG sample assayed also exhibited an amplicon of about 460 base pairs.

Sequence analysis of the about 460 base pair amplicon revealed that this amplicon form results from the splicing of exon 15 of the SCN11A hnRNA to exon 17; that is, the coding sequence of exon 16 is completely absent. This splice variant form was designated SCN11Asv1 [SEQ ID NO 5].

Additionally, RT-PCR primers were selected that were complementary to sequences with coordinates 23-45 and 1501-1528 of the reference exon coding sequences in SCN11A (AF188679). Based upon the nucleotide sequence of SCN11A mRNA, the SCN11A primer set with the coordinates 23-45 and 1501-1528 in SCN11A (AF188679) (hereafter ORF1+ primer set) was expected to amplify a 1548 base pair amplicon representing the “reference” SCN11A mRNA region. The ORF1+ forward primer at coordinates 23-45 in the SCN11A mRNA reference sequence has the sequence: 5′ TA TCGAAA TTAA TACGACTCACTATAGGGAG ACCCAAGCTGAGGGTGAAGATGGATGACAGATGC 3′ [SEQ ID NO 17]; and the ORF1+ reverse primer at coordinates 1501-1528 in the SCN11A mRNA reference sequence has the sequence: 5′ GTTTGGTTTGCTCTAGGAGCTGTGGCTT 3′ [SEQ ID NO 18]. The sequences in italics in the ORF1+ forward primer at coordinates 23-45 of the SCN11A mRNA are tail segments homologous to the cloning vector, used to facilitate subsequent recombination-based cloning steps (see Example 3).

The above described RT-PCR amplification was also performed on human DRG RNA using the ORF1+ primer set [SEQ ID NOs 17 and 18]. RT-PCR amplicons were size fractionated on a 2% agarose gel. Selected amplicon fragments were manually extracted from the gel and purified with a Qiagen Gel Extraction Kit. Purified amplicon fragments were cloned into an Invitrogen pCR2.1 vector using the reagents and instructions provided with the TOPO TA cloning kit (Invitrogen, Carlsbad, Calif.). Using the ORF1+ primers [SEQ ID NOs 17 and 18], the clones were screened by PCR and size fractionated on a 2% agarose gel to identify any variant insert sizes.

At least one different PCR amplicon was obtained from the clones using the ORF1+ primer set (data not shown). Most of the clones assayed exhibited the expected amplicon size of 1548 base pairs for normally spliced SCN11A mRNA. In addition, one clone assayed also exhibited an amplicon of about 1650 base pairs. Clones were then sequenced from each end (using the same primers used for RT-PCR) by Qiagen Genomics, Inc. (Bothell, Wash.).

Sequence analysis of the about 1650 base pair amplicon of SCN11A revealed that this amplicon form is due to the retention of a portion of intron 6, called intron 6A [SEQ ID NO 4] of the SCN11A hnRNA. That is, the longer form SCN11A amplicon is due to the insertion of intron 6A polynucleotide sequence [SEQ ID NO 4]. Thus, the RT-PCR results suggested that SCN11A mRNA in some tissue samples is composed of a mixed population of molecules wherein in at least two of the SCN11A mRNA splice junctions are altered.

### Example 2

### Cloning of SCN11Asv1 and SCN11Asv2

RT-PCR and sequencing data indicate that in addition to the normal SCN11A reference mRNA sequence, AF188679, encoding SCN11A protein, AAF17480, two novel splice variant forms of SCN11A mRNA also exist in DRG tissue.

Clones having a nucleotide sequence comprising the splice variants identified in Example 1 (hereafter referred to as SCN11Asv1 or SCN11Asv2) are isolated using recombination-mediated, PCR-directed plasmid construction in yeast. A set of four primer pairs were used to amplify the SCN11A coding region (AF188679) in four, sequential, overlapping segments (hereafter ORF1+, ORF2, ORF3, and ORF4). A 5′ “forward” ORF1+primer and a 3′ “reverse” ORF1+primer, a 5′ “forward” ORF2 primer and a 3′ “reverse” ORF2 primer, a 5′ “forward” ORF3 primer and a 3′ “reverse” ORF3 primer, and a 5′ “forward” ORF4 primer and a 3′ “reverse” ORF4 primer were made and used to amplify and clone the SCN11A mRNA (AF188679) coding sequences corresponding to ORF1+, ORF2, ORF3, and ORF4, respectively.

The 5′“forward” ORF1+ primer was designed to have the nucleotide sequence of 5′ TATCGAAATTAATACGACTCACTATAGGGAGACCCAAGCTGAGGGTGAAGATGGATG ACAGATGC 3′ [SEQ ID NO 17] and to have coordinates 23-45 relative to the SCN11A mRNA (AF188679). The 3′ “reverse” ORF1+ primer was designed to have the nucleotide sequence of 5′ GTTTGGTTTGCTCTAGGAGCTGTGGCTT 3′ [SEQ ID NO 18] and to have coordinates 1501-1528 relative to the SCN11A mRNA (AF188679).

The 5′ “forward” ORF2 primer was designed to have the nucleotide sequence of 5′ GTTGCCATGGGAATTGACAGAAGTTCAC 3′ [SEQ ID NO 19] and to have coordinates 1336-1363 relative to the SCN11A mRNA (AF1 88679). The 3′ “reverse” ORF2 primer was designed to have the nucleotide sequence of 5′ ATAGGCCTGTTGTTCAGGCTCAGGTTGT 3′ [SEQ ID NO 20] and to have coordinates 2844-2871 relative to the SCN11A mRNA (AF188679).

The 5′ “forward” ORF3 primer was designed to have the nucleotide sequence of 5′ ACTAACCTCTGTACCAAAGACCCTGGGC 3′ [SEQ ID NO 21] and to have coordinates 2727-2754 relative to the SCN11A mRNA (AF188679). The 3′ “reverse” ORF3 primer was designed to have the nucleotide sequence of 5′ CGTAAAGATGACCACAAAGACCCAGT TGA 3′ [SEQ ID NO 22] and to have coordinates 4232-4260 relative to the SCN11A mRNA (AF188679).

The 5′ “forward” ORF4 primer was designed to have the nucleotide sequence of 5′ AAAATGAGAAGAGTTAAACAGGTACGTAGCATGCCCGGGCGTGTTCGACATAGTCA CAAGCCAGA 3′ [SEQ ID NO 23] and to have coordinates 4104-4129 relative to the SCN11A mRNA (AF188679). The 3 ′ “reverse” ORF4 primer was designed to have the nucleotide sequence of 5′ GCATATTCAGATCCTCTTCTGAGATGAGTTTTTGTTCGAAAAGGCTGTGAA GCTATGAGGTAGGC 3′ [SEQ ID NO 24] and to have coordinates 5421-5445 relative to the SCN11A mRNA (AF188679).

The above-described primer sequences, primer coordinates relative to SCN11A mRNA (AF1 88679), and amplicon sizes are listed in Table 1. The primer sequences in italics are “tails” that were incorporated into the PCR amplicons and facilitate subsequent plasmid recombination events in yeast.

TABLE 1
PCR primers used to amplify SCN11A ORF13⁺ −4 segments

| | | | AF188679 | Amplicon |
|---|---|---|---|---|
| Primer name | SEQ ID NO | Primer sequence | coordinates | size |
| ORF1 + forward | SEQ ID NO 17 | TATCGAAATTAATACGACTCACTATAGGGAGAC | 23-45 | 1548 bp |
| | | CCAAGCTGAGGGTGAAGATGGATGACAGATGC | | |
| ORF1 + reverse | SEQ ID NO 18 | GTTTGGTTTGCTCTAGGAGCTGTGGCTT | 1501-1528 | 1548 bp |
| ORF2 forward | SEQ ID NO 19 | GTTGCCATGGGAATTGACAGAAGTTCAC | 1336-1363 | 1536 bp |
| ORF2 reverse | SEQ ID NO 20 | ATAGGCCTGTTGTTCAGGCTCAGGTTGT | 2844-2871 | 1536 bp |
| ORF3 forward | SEQ ID NO 21 | ACTAACCTCTGTACCAAAGACCCTGGGC | 2727-2754 | 1534 bp |
| ORF3 reverse | SEQ ID NO 22 | CGTAAAGATGACCACAAAGACCCAGTTGA | 4232-4260 | 1534 bp |
| ORF 4 forward | SEQ ID NO 23 | AAAATGAGAAGAGTTAAACAGGTACGTAGCAT | 4104-4129 | 1341 bp |
| | | GCCCGGGCGTGTTCGACATAGTCACAAGCCAGA | | |
| ORF4 reverse | SEQ ID NO 24 | GCATATTCAGATCCTCTTCTGAGATGAGTTTTT | 5421-5445 | 1341 bp |
| | | GTTCGAAAAGGCTGTGAAGCTATGAGGTAGGC | | |

RT-PCR

Four segments of SCN11A cDNA sequence corresponding to ORF1+, ORF2, ORF3, and ORF4 were cloned using a combination of reverse transcription (RT) and polymerase chain reaction (PCR). More specifically, about 25 ng of human dorsal root total RNA (BD Biosciences Clontech, Palo alto, Calif.) was reverse transcribed using Superscript II (Gibco/Invitrogen, Carlsbad, Calif.) and oligo d(T) primer (RESGEN/Invitrogen, Huntsville, Ala.) according to the Superscript II manufacturer's instructions. For PCR, 1 μl of the completed RT reaction was added to 40 μl of water, 5 μl of 10× buffer, 1 μl of dNTPs and 1 μl of enzyme from the Clontech (Palo Alto, Calif.) Advantage 2 PCR kit. PCR was done in a Gene Amp PCR System 9700 (Applied Biosystems, Foster City, Calif.) using the SCN11A ORF1+, ORF2, ORF3, or ORF4 “forward” and “reverse” primers [SEQ ID NOs 17-24]. After an initial 94° C. denaturation of 1 minute, 35 cycles of amplification were performed using a 30 second denaturation at 94° C. followed by a 40 second annealing at 63.5° C. and a 50 second synthesis at 72° C. The 35 cycles of PCR were followed by a 10 minute extension at 72° C. The 50 μl reaction was then chilled to 4° C. 10 μl of the resulting reaction product was run on a 1% agarose (Invitrogen, Ultra pure) gel stained with 0.3 μg/ml ethidium bromide (Fisher Biotech, Fair Lawn, N.J.). Nucleic acid bands in the gel were visualized and photographed on a UV light box to determine if the PCR had yielded products of the expected size, in the case of the predicted ORF1+ mRNA, a product of about 1548 base pairs, in the case of the predicted ORF2 mRNA, a product of about 1536 base pairs, in the case of ORF3 mRNA, a product of about 1534 base pairs, and in the case of ORF4, a product of about 1341 base pairs. The remainder of the 50 μl PCR reactions from DRG was purified using the QIAquik Gel extraction Kit (Qiagen, Valencia, Calif.) following the QIAquik PCR Purification Protocol provided with the kit. About 50 μl of product obtained from the purification protocol was concentrated to about 6 μl by drying in a Speed Vac Plus (SC110A, from Savant, Holbrook, N.Y.) attached to a Universal Vacuum System 400 (also from Savant) for about 30 minutes on medium heat.

Cloning of RT-PCR Products

About 4 μl of the 6 μl of purified ORF1+, ORF2, ORF3, and ORF4 RT-PCR product from dorsal root ganglia were cloned into an Invitrogen pCR2.1 vector using the reagents and instructions provided with the TOPO TA cloning kit (Invitrogen, Carlsbad, Calif.). About 2 μl of the cloning reaction was used following the manufacturer's instructions to transform TOP10 chemically competent E. coli provided with the cloning kit. After the 1 hour recovery of the cells in SOC medium (provided with the TOPO TA cloning kit), 200 μl of the mixture was plated on LB medium plates (Sambrook, et al., in Molecular Cloning, A Laboratory Manual, 2^{nd} Edition, Cold Spring Harbor Laboratory Press, 1989) containing 100 μg/ml Ampicillin (Sigma, St. Louis, Mo.) and 80 μg/ml X-GAL (5-Bromo-4-chloro-3-indoyl B-D-galactoside, Sigma, St. Louis, Mo.). Plates were incubated overnight at 37° C. White colonies were picked from the plates into 2 ml of 2×LB medium. These liquid cultures were incubated overnight on a roller at 37° C. Plasmid DNA was extracted from these cultures using the Qiagen (Valencia, Calif.) Qiaquik Spin Miniprep kit.

Four putative ORF1+, ORF2, and ORF3 clones were identified and prepared for a PCR reaction to confirm the presence of the expected ORF1+, ORF2, and ORF3 structure. A 25 μl PCR reaction was performed as described above (RT-PCR section) to detect the presence of ORF1+, ORF2, and ORF3, except that the reaction included miniprep DNA from the TOPO TA/ORF1+, TOPO TA/ORF2, and TOPO TA/ORF3 ligation, respectively, as a template. About 10 μl of each 25 μl PCR reaction was run on a 1% Agarose gel and the DNA bands generated by the PCR reaction were visualized and photographed on a UV light box to determine which minipreps samples have PCR product of the size predicted for the corresponding ORF1+, ORF2, and ORF3 mRNA. Clones having the ORF1+, ORF2, and ORF3 structure were identified based upon amplification of an amplicon band of 1548, 1536, and 1534 base pairs, respectively. DNA sequence analysis of the ORF1+, ORF2, and ORF3 cloned DNA confirmed a polynucleotide sequence representing the ORF1+, ORF2, and ORF3 based upon SCN11A mRNA (AF188679). TOPO TA/ORF4 clones could not be isolated, so the purified RT-PCR ORF4 product was cloned directly into the yeast plasmid as described in subsequent paragraphs.

The TOPO TA/ORF1+, TOPO TA/ORF2, and TOPO TA/ORF3 ligations were used as template for a brief (≦10 cycle) PCR as described above (RT-PCR section) using ORF1+ primers [SEQ ID NOs 17 and 18], ORF2 primers [SEQ ID NOs 19 and 20], and ORF3 primers [SEQ ID NOs 21 and 22], respectively, to create sufficient quantities of the amplicons for recombination.

Construction of Cycloheximide-Resistant Saccharomyces cerevisiae Strain

A cycloheximide-based counterselection was used to increase specificity of cloning by homologous recombination relative to nonspecific vector background (Raymond et al, 2002, Genome Res. 12:190-197). Plasmid re-circularization is the primary source of background in recombinational cloning experiments (Boulton and Jackson, 1996, Nucleic Acids Res. 24:4639-4648; Raymond et al., 1999, Biotechniques 26:134-8, 140-1). The yeast strain used in this study, CMY1-5, is cycloheximide resistant (CYH2^{R}). The introduction of the wild-type CYH2 allele into this yeast strain confers dominant sensitivity to cycloheximide. The position of the CYH2 gene in the cloning vector relative to the targeted recombination sites was designed such that the CYH2 gene was lost in recombinant clones but retained in most end-joined plasmids. Therefore, yeast cells containing recombinant plasmids were selected in the presence of cycloheximide, while yeast cells containing non-recombinant plasmids were sensitive to the drug. However, any marker that confers dominant sensitivity could be used in a counterselection experiment, such as URA3, 5-fluoroorotic acid, LYS2 and α-amino adipic acid, or CAN1.

A cycloheximide resistant strain was generated from the cycloheximide sensitive yeast strain BY4709 (Brachmann et al, 1998, Yeast 14:155-132). Two overlapping 1 kb products from the CYH2 gene were amplified from BY4709 yeast strain using two sets of tailed primers (hereafter Set 1 and Set 2) listed in Table 2, which introduce a critical Q→E amino acid change at residue 38 in the Cyh2p protein, thereby conferring drug resistance. The 5′ “forward” Set 1 primer was designed to have the nucleotide sequence of 5′ ATACGACTCACTATAGGG AGACCCAAGCTGGCTAGTTAAGTATGTTTATATATGGATTTTGAAA 3′ [SEQ ID NO 25]. The 3′ “reverse” Set 1 primer was designed to have the nucleotide sequence of 5′ GTA GAGGTATGGCCGGTGGTGAACATCACCACAGAATTAAC 3′ [SEQ ID NO 26]. The 5′ “forward” Set 2 primer was designed to have the nucleotide sequence of 5′ TCCACCACACTG GACTAGTGGATCCGAGCTCGGTACCAAGGCCCGGGCATGCTACGTACCTGTTTAACTCTTC 3′ [SEQ ID NO 27]. The 3′ “reverse” Set 2 primer was designed to have the nucleotide sequence of 5′ GTTAATTCTGTGGTGATGTTCACCACCGGCCATACCTCTAC 3′ [SEQ ID NO 28].

TABLE 2

Tailed PCR primer sets used to amplify CYH2
and introduce a drug-resistant mutation

| | | |
|---|---|---|
| Primer Name | SEQ ID NO | Primer Sequence |

| | | |
|---|---|---|
| Set 1 Forward | SEQ ID NO 25 | ATACGACTCACTATAGGGAGACCC |
| | | AAGCTGGCTAGTTAAGTATGTTTA |
| | | TATATGGATTTTGAAA |
| Set 1 Reverse | SEQ ID NO 26 | GTAGAGGTATGGCCGGTGGTGAAC |
| | | ATCACCACAGAATTAAC |
| Set 2 Forward | SEQ ID NO 27 | TCCACCACACTGGACTAGTGGATC |
| | | CGAGCTCGGTACCAAGGCCCGGGC |
| | | ATGCTACGTACCTGTTT |
| | | AACTCTTC |
| Set 2 Reverse | SEQ ID NO 28 | GTTAATTCTGTGGTGATGTTCACC |
| | | ACCGGCCATACCTCTAC |

Recombination of the Set 1 and Set 2 CYH2 amplicons into the endogenous yeast CYH2 gene confers cycloheximide resistance. 1 μg each of the Set 1 and Set 2 PCR amplicons, which incorporate the Q38E mutation, were cotransformed by electroporation as described in Raymond et al. (2002, Genome Res. 12:190-197) with yeast strain BY4709 (Brachmann et al., 1998 Yeast, 14:115-132), and cycloheximide resistant colonies were selected on media containing 1 μg/ml cycloheximide (Sigma, St. Louis, Mo.). Yeast transformation methods, including electroporation, lithium acetate treatment, and spheroplasting, are also discussed in Gietz and Woods (2001 Biotechniques, 30:816-820, 822-826, 828). One cycloheximide resistant strain, CMY1-5 (Mato, ura3Δ, cyh2^{R}) was used for all subsequent studies.

Construction of Yeast Plasmids

Plasmids that are assembled in yeast by recombination typically include sequence elements that allow their selection in yeast (e.g., CYH2 or URA3 resistance). Sequence elements that permit plasmid replication include a yeast centromere sequence and yeast DNA autonomously replicating sequence. DNA sequences for selection (e.g., ampicillin, kanamycin, or chloramphenicol resistance) and replication (e.g., colE1 or mini F′) in Escherichia coli are typically included in the plasmid to allow transformation of E. coli for the preparation of large quantities of recombinant plasmid. Isolation of yeast plasmid for bacterial transformation is described in Hoffman and Winston (1987, Gene 57:267-72). One such embodiment of a yeast plasmid is the sequences found in the vector pRS316 (Sikorski and Hieter, 1989, Genetics 122:19-27). Additional elements including promoters, terminators, and selectable markers for recombinant expression in mammalian cells can be found in commercially available plasmid vectors and incorporated into yeast cloning vectors.

Overlapping sequences are used to target recombinational cloning of DNA fragments into specific sites in the target yeast plasmid. The region of overlap can be as short as 20 bp, but is optimally 40 bp or longer (Hudson et al, 1997, Genome Res. 7:1169-1173; Oldenburg et al., 1997 Nucleic Acids Res. 25:451-452; Hua et al., 1997, Plasmid 38:91-96; Raymond et al, 1999, Biotechniques, 26:134-8, 140-1). Sequence homology for these overlapping regions may be provided by amplification of target sequences with PCR primers that include varying lengths of base pair extensions (“tails”) that become incorporated into the amplicons. Alternatively, synthetic oligonucleotide recombination linkers that provide sequence homology at each end to the two unrelated DNA molecules to be joined may be used (Raymond et al, 2002, Genome Res. 12:190-197; DeMarini et al., 2001, Biotechniques 30:520-523).

To create the cloning vectors used for this study, plasmid pRS416 (ATCC No. 87521) (Sikorski and Hieter, 1989, Genetics, 122:19-27) was digested with SspI; and plasmid pENTR11 (InVitrogen, Carlsbad, Calif.) was digested with NheI. BY4709 yeast strain was cotransformed with the two plasmid vector fragments and the recombinational linkers listed in Table 3 [SEQ ID NOs 29-32] by electroporation (Raymond et al., 2002 Genome Res. 12:190-197) to form the resulting pCMR2 plasmid [SEQ ID NO 33]. All subsequent transformation steps for plasmid construction were performed with yeast strain BY4709 using the referenced electroporation method unless otherwise indicated.

TABLE 3

Linkers used to joining pRS416 (SspI)
and pENTR11 (NheI).

| | |
|---|---|
| SEQ ID NO | Linker Sequence |

| | |
|---|---|
| SEQ ID NO 29 | TGCGTTATCCCCTGATTCTGTGGATAACCGTATTACC |
| | GCTGGGTAATAACTGATATAATTAAATTGAAG |
| | CTCTAATTTGT |
| SEQ ID NO 30 | ACAAATTAGAGCTTCAATTTAATTATATCAGTTATTA |
| | CCCAGCGGTAATACGGTTATCCACAGAATCAGG |
| | GGATAACGCA |
| SEQ ID NO 31 | AATTTAAATTATAATTATTTTTATAGCACGTGATGAA |
| | AAGAGCATGGATCTCGGGGACGTCTAACTACTA |
| | AGCGAGAGTA |
| SEQ ID NO 32 | TACTCTCGCTTAGTAGTTAGACGTCCCCCGAGATCCA |
| | TGCTCTTTTCATCACGTGCTATAAAAATAATTAT |
| | AATTTAAATT |

pCMR2 plasmid [SEQ ID NO 33] was cut with SmaI and then recombined with the linkers in Table 4 [SEQ ID NO 34, 35] to produce pCMR3 [SEQ ID NO 36].

TABLE 4
Linkers used to convert pCMR2 to pCMR3.

| | |
|---|---|
| SEQ ID NO | Linker Sequence |

| | |
|---|---|
| SEQ ID NO 34 | ACTTTGTACAAAAAAGCAGGCTTCGAAGGAGATAGAA |
| | CCAGCCCGGGCGCCGCACTCGAGATATCTAGACCCAG |
| | CTTTCTTGTACAAA |
| SEQ ID NO 35 | TTTGTACAAGAAAGCTGGGTCTAGATATCTCGAGTGC |
| | GGCGCCCGGGCTGGTTCTATCTCCTTCGAAGCCTGCT |
| | TTTTTGTACAAAGT |

pCMR3 [SEQ ID NO 36] was digested with SspI, and pCCFOS1 (EpiCentre Technologies, Madison, Wis.) was cut with SalI. The vector fragments were then joined with the linkers shown in Table 5 [SEQ ID NOs 37-40], resulting in plasmid pCMR7 [SEQ ID NO 41].

TABLE 5
Linkers used to join pCMR3 and pCCFOS1.

| | |
|---|---|
| SEQ ID NO | Linker Sequence |

| | |
|---|---|
| SEQ ID NO 37 | GTTAACCGGGCTGCATCCGATGCAAGTGTGTCGCTGT |
| | CGAGGGTAATAACTGATATAATTAAATTGAAGCTCT |
| | AATTTTGT |
| SEQ ID NO 38 | ACAAATTAGAGCTTCAATTTAATTATATCAGTTATTA |
| | CCCTCGACAGCGACACACTTGCATCGGATGCAG |
| | CCCGGTTAAC |
| SEQ ID NO 39 | ATAAAATCATTATTTGCCATCCAGCTGCAGCTCTGGC |
| | CCGTCGAATTTCTGCCATTCATCCGCTTATTAT |
| | CACTTATTCA |
| SEQ ID NO 40 | TGAATAAGTGATAATAAGCGGATGAATGGCAGAAATT |
| | CGACGGGCCAGAGCTGCAGCTGGATGGCAAATA |
| | ATGATTTTAT |

Plasmid pCMR7 [SEQ ID NO 41] was cut with SrfI; plasmid pcDNA3.1 mycHIS A (InVitrogen) was cut with SspI. The resulting plasmid fragments were joined with the linkers shown in Table 6 [SEQ ID NOs 42-45], yielding plasmid pCMR9 [SEQ ID NO 46].

TABLE 6
Linkers used to join pCMR7 and pcDNA3.1 mycHIS A.

| | |
|---|---|
| SEQ ID NO | Liner Sequence |

| | |
|---|---|
| SEQ ID NO 42 | ACGGTTATCCACAGAATCAGGGGATAACGCAGGAAAG |
| | AACATAAAATCATTATTTGCCATCCAGCTGCAG |
| | CTCTGGCCCG |
| SEQ ID NO 43 | CGGGCCAGAGCTGCAGCTGGATGGCAAATAATGATTT |
| | TATGTTCTTTCCTGCGTTATCCCCTGATTCTGT |
| | GGATAACCGT |
| SEQ ID NO 44 | AATTTAAATTATAATTATTTTTATAGCACGTGATGAA |
| | AAGTCCGCGCACATTTCCCCGAAAAGTGCCACC |
| | TGACGTCGAC |
| SEQ ID NO 45 | GTCGACGTCAGGTGGCACTTTTCGGGGAAATGTGCGC |
| | GGACTTTTCATCACGTGCTATAAAAATAATTAT |
| | AATTTAAATT |

Construct pCMR9 [SEQ ID NO 46] was cut with HindIII. The yeast CYH2 gene was amplified as two 1 kb overlapping pieces from yeast strain BY4709 (Brachmann et al, 1998, Yeast 14:155-132), using the two sets of tailed primers listed in Table 7. The sequences of one set of CYH2 primers are set forth in SEQ ID NOs 25 and 47; the sequences of the second set of primers are set forth in SEQ ID NOs 27 and 48. The full length CYH2 gene was assembled into pCMR9 by cotransformation of CMY1-5 yeast strain to produce pCMR11 [SEQ ID NO 49] (see Table 8).

TABLE 7

Tailed PCR primers used to amplify
CYH2 for recombination into pCMR9.

| | |
|---|---|
| SEQ ID NO | Primer Sequence |

| | |
|---|---|
| SEQ ID NO 25 | ATACGACTCACTATAGGGAGACCCAAGCTGGCTAGTT |
| | AAGTATGTTTATATATGGATTTTGAAA |
| SEQ ID NO 47 | GTAGAGGTATGGCCGGTGGTCAACATCACCA |
| | CAGAATTAAC |
| SEQ ID NO 27 | TCCACCACACTGGACTAGTGGATCCCAGCTCGGTACC |
| | AAGGCCCGGGCATGCTACGTACCTGTTTAACTCTTC |
| SEQ ID NO 28 | GTTAATTCTGTGGTGATGTTGACCACCGGCC |
| | ATACCTCTAC |

TABLE 8
Composition of pCMR10 and pCMR11 plasmids

| Nucleotide | |
|---|---|
| coordinates | Functional description of sequence |
| 1-6013 | Copy-control ™ E. coli origin of replication from |
| | pCC1FOS (Epicentre Technologies, Madison, WI). |
| 6014-7884 | Yeast URA3 gene, ARS4 autonomously replicating |
| | sequence and CEN6 centromere |
| | from pRS316 (Sikorski and Heiter, 1989). |
| 7885-8825 | Mammalian CMV promoter from InVitrogen |
| | (Carlsbad, CA) vector pcDNA3.1/myc- |
| | HIS A. |
| 8826-10,774 | Yeast CYH2 gene amplified from strain |
| | BY4709 (Brachmann et al. 1998) |
| 10,775-10,782 | Engineered SrfI restriction site. |
| 10,783-13,556 | Mammalian poly-adenylation sites, selectable |
| | markers, SV40 origin, etc. from |
| | pcDNA3.1/myc-HIS A. |
| 13,557-13,596 | DNA sequence from InVitrogen vector pENTR11. |
| 13,597-14,567 | pCMR10 - specific; kanamycin resistance |
| | gene from pENTR11. |
| 13,597-14,561 | pCMR11 - specific; chloramphenicol resistance |
| | gene from pCC1FOS. |

Plasmid pCMR11 [SEQ ID NO 49] carries a chloramphenicol resistance gene. To convert this resistance gene to a kanamycin resistance marker, pCMR11 [SEQ ID NO 49] was digested with BspEI. The kanamycin resistance gene was amplified from pENTR11 (InVitrogen) with the tailed primers shown in Table 9 [SEQ ID NOs 50 and 51]. The digested pCMR11 [SEQ ID NO 49] and kanamycin amplicon were joined by recombination in yeast strain CMY1-5. The resulting plasmid was called pCMR10 [SEQ ID NO 52] (see Table 8).

TABLE 9

Tailed PCR primers used to amplify
the kanamycin resistance marker from
pENTR11 for recombination into pCMR11.

| | |
|---|---|
| SEQ ID NO | Primer sequence |

| | |
|---|---|
| SEQ ID NO 50 | ATAAAATCATTATTTGCCATCCAGCTGCAGCTCTGGC |
| | CCGTGTCTCAAAATCTCTGATGTTACAT |
| SEQ ID NO 51 | TTTCTCTGTCCTTGCCTGTGCGACGGTTACGCCGCTC |
| | CATGGTCTGACGCTCAGTGGAACGGGGCC |

Assembly of SCN1Asv1 and SCN11Asv2 Full-Length Clones and Yeast Transformation

In addition to sequence homology between the two sequences to be joined, the quantities of acceptor vector and donor PCR fragments are critical for efficient recombination. As described in Raymond et al. (2002, Genome Res. 12:190-197), 100 ng of acceptor vector and 1 μg of DNA donor fragment were used to transform yeast cells. Assembly of the full-length SCN11Asv1 and SCN11Asv2 full length clones by homologous recombination between overlapping pieces of ORF1+, ORF2, ORF3, ORF4, a DNA fragment representing the region of the alternative splicing event, and the expression vector was performed by simultaneous transformation of these pieces into yeast cells. All yeast transformation steps described in subsequent paragraphs were performed by electroporation (Raymond et al., 2002 Genome Res. 12:190-197). Yeast transformation methods (electroporation, lithium acetate treatment, or spheroplasting) are also compared by Gietz and Woods (2001, Biotechniques 30:816-820, 822-826, 828).

1 μg ORF4 amplicon was cloned directly into 100 ng of pCMR11 [SEQ ID NO 49] by cotransformation of 100 μl of yeast strain CMY1-5 (Mata, URA3Δ, CYH2R). Ura+, cycloheximide resistant colonies were selected on Ura-deficient media plates containing 1 μg/ml cycloheximide (Sigma, St. Louis, Mo.). Standard yeast media were used (Sherman, 1991, Methods Enzymol. 194:3-21).

To construct the SCN11Asv1 clone, 1 μg each of ORF130 , ORF2, and SacI-digested ORF3 fragments that had undergone a 10-cycle PCR, as described previously, 1 μg of an EcoRI-XmnI DNA fragment that spans the region of the alternative splicing of exon 15 to exon 17 [SEQ ID NO 53], and 100 ng of SrfI-digested ORF4/pCMR11 clone are used to cotransform 100 μl of CMY1-5 yeast strain. The EcoRI-XmnI DNA fragment that spans the exon 16 drop event is made by restriction digestion of the TOPO TA clone identified in Example 1. The first 12 polynucleotides of SEQ ID NO 53 are vector encoded. The overlapping DNA between the sequential DNA fragments dictates that most yeast transformants will possess the correctly assembled construct. Ura+, cycloheximide resistant colonies are selected for subsequent preparation and transformation of E. coli. Plasmid DNA extracted from E. coli is analyzed by restriction digest to confirm the presence of the alternative splicing of exon 15 to exon 17 in the SCN11Asv1 clone. Eight SCN11Asv1 clones are sequenced to confirm identity, and the clones corresponding to pCMR17 [SEQ ID NO 54] are used for protein expression in multiple systems, including mammalian cells.

To construct the SCN11Asv2 clone, 1 μg each of NciI-digested ORF1+, ORF2, ORF3 fragments that had undergone a 10 cycle PCR, as described previously, 1 μg of an amplicon that spans the region of the intron 6A insertion, and 100 ng of SrfI-digested ORF4/pCMR11 clone are used to cotransform 100 μl of CMY1-5 yeast strain. The DNA fragment containing the intron 6A insertion is amplified from the intron 6A insertion clone from Example 1 using intron 6A insertion forward and reverse primers [SEQ ID NOs 55 and 56] shown in Table 10. Ura+, cycloheximide resistant colonies are selected for subsequent preparation and transformation of E. coli. Plasmid DNA extracted from E. coli is analyzed by restriction digest to confirm the presence of the intron 6A insertion in the SCN11Asv2 clone. Eight SCN11Asv2 clones are sequenced to confirm identity, and the clones corresponding to pCMR18 [SEQ ID NO 57] are used for protein expression in multiple systems, including mammalian cells.

TABLE 10

Primers used to amplify the alternative
splicing of SCN11Asv2.

| | | |
|---|---|---|
| Primer name | SEQ ID NO | Primer sequence |

| | | |
|---|---|---|
| Intron 6A | SEQ ID NO 55 | TATCGAAATTAATACGACT |
| insertion forward | | CACTATAGGGAGACCCAAG |
| | | CTGAGGGTGAAGATGGATG |
| | | ACAGATGC |
| Intron 6A | SEQ ID NO 56 | CCATTGCTTTGAAAAGAAA |
| insertion reverse | | GAAAATTCACCTG |
| | | AATTCAAA |

The polynucleotide sequence of SCN11Asv1 mRNA [SEQ ID NO 5] lacks a 114 base pair region corresponding to exon 16 of the full length coding sequence of the reference SCN11A mRNA (AF188679). Deletion of the 114 base pair region does not alter the protein translation reading frame. Therefore, the SCN11Asv1 polypeptide is lacking an internal 38 amino acid region corresponding to exon 16 of the full length coding sequence of the reference SCN11A mRNA (AF188679).

The polynucleotide sequence of SCN11Asv2 mRNA [SEQ ID NO 7] contains an additional 102 nucleic acids region encoded within intron 6 of the full length SCN11A gene, referred to as intron 6A [SEQ ID NO 4]. The 102 nucleotide insertion does not alter the protein translation reading frame. Therefore, the SCN11Asv2 polypeptide contains 34 additional amino acids that are not present in the full length coding sequence of the reference SCN11A mRNA (AF188679).

### Example 3

### Real-Time Quantitative PCR/TAQman

To determine the relative mRNA abundances of SCN11Asv1 alternatively spliced isoform to the SCN11A reference protein (AAF17480), a real-time quantitative PCR assay was used. Materials and methods for quantification of splice variants using real-time PCR, using boundary specific probes are known in the art (Kafert et al., 1999 Anal. Biochem. 269:210-213; Vandenbroucke et al, 2001 Nucleic Acids Res. 29:E68-8; Taveau et al., 2002 Anal. Biochem. 305:227-235).

Reverse Transcription

RNA samples from human DRG (ClonTech, Palo Alto, Calif.) were reverse transcribed using the Applied Biosystems (Foster City, Calif.) TAQman reverse transcription kit N808-0234 following manufacturer's instructions. A 50 μl reaction contained:

| | |
|---|---|
| 5 | μl 10X RT buffer |
| 11 | μl MgCl₂ solution |
| 10 | μl dNTP solution |
| 2.5 | μl random hexamer primer |
| 1 | μl RNAse OUT |
| 3 | μl Multiscribe reverse transcriptase |
| 1 | μg of RNA |

H₂O to a final volume of 50 μl.

To convert RNA to single-stranded cDNA, the reaction mixture was incubated at the following conditions: 25° C. for 10 minutes, 37° C. for 60 minutes, 95° C. for 5 minutes. The cDNA sample was then placed on ice prior to use.

Plasmid Construction and Standard Curve

Plasmids carrying the reference SCN11A sequence and alternatively spliced isoform SCN11Asv1 were constructed in order to prepare a standard curve. The SCN11A cDNA region spanning nucleotides from exon 15 to exon 19 was amplified with exon 15 primer 5′ GATGACGTTGAATTTTCTGGTGAAGATA 3′ [SEQ ID NO 15] and exon 19 primer 5′ CAAATCCGAAGGCTACCCATTTTAGTA 3′ [SEQ ID NO 16] from DRG cDNA. The PCR products were cloned into pCR2.1 vector (Invitrogen). The cloning reaction was used to transform TOP10 chemically competent E. coli cells, and plasmid DNA was extracted using the Qiagen (Valencia, Calif.) Qiaquick Spin Miniprep kit. DNA was quantified using a UV spectrometer. Sequence identities of plasmid clones containing the SCN11A reference sequence and alternatively spliced SCN11Asv1 sequence, which lacks exon 16, were verified.

To construct a standard curve with the plasmid clones carrying the SCN11A reference sequence and SCN11Asv1 sequence, ten-fold serial dilutions of the plasmids were used to obtain a range of five orders of magnitude. Final plasmid concentrations of 100 pg, 10 pg, 1 pg, 0.1 pg, and 0.01 pg were amplified using real-time PCR. Fluorescence emission values were plotted onto a standard curve, permitting quantification of the experimental samples compared to the standard curve.

Real-Time PCR

TAQman primers and probes used to quantify the SCN11Asv1 isoform were designed and synthesized as pre-set mixtures (Applied Biosystems, Foster City, Calif.). The sequences of the TAQman primers and probes used to quantify the SCN11A reference form [SEQ ID NOs 58, 59 and 60] and SCN11Asv1 isoform [SEQ ID NOs 61, 62 and 63] are shown in Table 11. Splice junction specific probes were labeled with the 6-FAM fluorphore at the 5′ end (FAM) and a non-fluorescent quencher at the 3′ end (NFQ). Real-time PCR was performed on human DRG cDNA using the TaqMan Universal PCR Master Mix (Applied Biosystems, Foster City, Calif.). The TAQman reaction contained:

| 96-well format | 384-well format | |
|---|---|---|
| 12.5 μl | 5 μl | TAQman Universal MasterMix |
| 1.25 μl | 0.5 μl | Primer-probe mix |
| 6.25 μl | 2.5 μl | H₂O |
| 5 μl | 2 μl | cDNA or plasmid DNA. |

TABLE 11
Primers and probes used to quantify SCN11A isoforms.

| | | | |
|---|---|---|---|
| Name | SEQ ID NO | Sequence | Specificity |

| | | | |
|---|---|---|---|
| SCN11A reference forward primer | SEQ ID NO 58 | TCACACAACCTGAGCCTGAAC | SCN11A reference |
| SCN11A reference reverse primer | SEQ ID NO 59 | GTGGGCTTCTTGTTCTCCTGAT | SCN11A reference |
| SCN11A reference probe | SEQ ID NO 60 | FAM-AACAGGCCTATGAGCTCC-NFQ | SCN11A reference |
| SCN11Asv1 forward primer | SEQ ID NO 61 | ACAGCGCATCACACAACCT | SCN11Asv1 |
| SCN11Asv1 reverse primer | SEQ ID NO 62 | CTGAAGATCAATGGTGCTACATTCTG | SCN11Asv1 |
| SCN11Asv1 probe | SEQ ID NO 63 | FAM-CCTGAACAACAGAAGTCT-NFQ | SCN11Asv1 |

The TAQman reactions were performed on an ABI Prism 7900HT Sequence Detection System (Applied Biosystems, Foster City, Calif.). The thermocycling conditions were 50° C. for 2 minutes, 95° C. for 10 minutes, and 40 cycles of 95° C. for 15 seconds and 60° C. for 1 minute. Data analysis of the fluorescence emission was performed by the Sequence Detector Software (SDS) (Applied Biosystems, Foster City, Calif.). Briefly, an amplification plot was generated for each sample, which showed cycle number on the x axis vs. ΔRn on the y axis. Rn is the fluorescence emission intensity of the reporter dye normalized to a passive reference, and ΔRn is the Rn value of the reaction minus the Rn of an un-reacted sample. A threshold cycle (C_{T}) value, the cycle at which a statistically significant increase in ΔRn is first detected, was calculated from the amplification plot. The threshold was automatically calculated by the SDS as the 10-fold standard deviation of the Rn in the first 15 cycles. The obtained C_{T} values were exported Microsoft Excel for analysis as recommended by the manufacturer (Applied Biosystems, Foster City, Calif.). Standard curve plots showing the log₁₀ [input cDNA] vs. C_{T} values were constructed. Referring to the standard curve, C_{T} values for the experimental samples were then used to calculate the input amount of the SCN11A isoform cDNA. The most highly expressed isoform, in this case the reference form of SCN11A, was assigned the arbitrary value of 100%, and other isoforms were presented as percentages the most highly expressed isoform. Quantitative analysis of the real-time PCR data indicated that the SCN11Asv1 isoform is relatively rare in human DRG, at a ratio of 1:59 compared to the reference SCN11A.

### Example 4

### Analyzing Ion Channel Activity of SCN11A and Other Sodium Channel Isoforms

To express SCN11A and other sodium channel isoform channels in Xenopus oocytes, cDNA encoding the appropriate channel is cloned into pCMR11 [SEQ ID NO 49] by recombination in yeast and subcloned into standard expression vectors, such as pClneo (Promega) or pcDNA3 (Invitrogen), or into a modified pGEM vector (Promega) containing the Xenopus β-globin 5′ and 3′ untranslated region to enhance expression levels as described in Goldin, A. L. (1991, Methods Cell Biol. 36: 487-509). Plasmids are linearized, and RNA is transcribed using the T7 (or SP6) RNA polymerase and the mMessage mMachine kit from Ambion. RNA is either directly injected or diluted prior to injection to obtain maximum current amplitudes of between 0.5 and 5 μA at test potentials 2-7 days after mRNA injection. Current amplitude is measured using a Dagan two-electrode voltage-clamp (TEVC) amplifier (Dagan Instruments Minneapolis, Minn.) and the pCLAMP data acquisition software (Axon Instruments, Foster City, Calif.). Leak current is subtracted using the scaled current observed with a P/n protocol (Benzanilla and Armstrong, 1977 J. Gen. Physiol. 70:549-566). The capacitance and resistance compensation feature on the Dagan TEVC amplifier is used to minimize the capacitance transient measured at a voltage where Nav channels are not opened. Glass microelectrodes are pulled to achieve resistances of 0.5-1.0MΩ after filling with 1 M KCl measured in the recording solution. Oocytes are prepared by standard techniques and recording is done in ND-96 (96 mM NaCl, 2 mM KCl, 1 mM MgCl₂, 1.8 mM CaCl₂, 5 mM HEPES-Na, pH 7.5). Oocyte membrane potential is held at −80 or −100 mV, and a current-voltage relationship is measured to determine the voltage that produced the maximum inward current. Typically, oocytes are depolarized to 0 mV for 40-100 ms. After a stable peak current is obtained, the indicated concentration of venom, toxin, or compound is added by perfusing the 1 mL recording chamber with ND 96 containing the sample, typically at 2-3 mL/min until stable block is achieved. The oocyte is subsequently perfused with fresh ND-96 to assess reversibility of inhibition.

Cell Culture and HEK Cell Electrophysioloy:

For whole-cell voltage-clamp recordings, stably transfected HEK-293 cells or other eukaryotic cell lines expressing SCN11A or other sodium channel isoforms are used. Retro-virus vectors may be used to generate cell lines expressing SCN11A or other sodium channel isoform cell lines. The SCN11A or other sodium channel isoform is cloned in a retroviral expression vector (pLCNX, Clontech). Subsequently virus particles are used to infect HEK-293 cells, and a cell line stably expressing the SCN11A channel is selected. Cells are maintained in either MEM (Minimum Essential Medium) supplemented with 10% fetal bovine serum, 2 mM L-glutamine, 1 unit/mL penicillin/streptomycin or DMEM (Dulbecco's modified Eagles medium) supplemented with 10% fetal bovine serum, 4 mM L-glutamine, 2 units/mL penicillin/streptomycin, 0.02 mg/mL G-418, as appropriate. Cells are plated on poly-D-lysine coated cover slips 16 hours prior to recording and are then washed with mammalian Ringer's solution immediately prior to recording.

Single cell recordings of currents through voltage-activated SCN11A or other sodium channel isoforms are performed at room temperature (20-22° C.) using the whole cell patch-clamp technique (Hamill et al., 1981, Pflugers Archives 391: 85-100). Currents are recorded using a Dagan 3900A (Dagan Instruments, Minneapolis, Minn.) or HEKA EPC-9 *HEKA Electronics, Lambrecht, Germany) patch clamp amplifier. Data are stored on a personal computer equipped with HEKA Pulse 8.5 and analyzed using Pulsefit (HEKA Electronics, Lambrecht, Germany), Igor Pro 4.0 (Wavemetrics, Lake Oswego, Oreg.), or Origin 6.0 (Microcal, Northampton, Mass.). Patch pipets are made from borosilicate glass tubing (World Precision Instruments, Sarasota, Fla.), fire-polished, and coated with Sylgard and have a resistance of 1-3MΩ when filled with an internal solution (below) measured in the recording medium. Series resistance and capacitance are compensated using the amplifier's circuitry. Leak resistance is corrected by subtracting the scaled current observed with a P/n protocol. For sodium current measurements, the following solutions are used (in mM): 1) Pipet Solution A: 2 NaCl, 101 Cs·gluconate, 20 CsF, 20 CsCl, 11 BAPTA, pH 7.4 adjusted with CsOH; or 2) Pipet Solution B: 140 CsF, 10 NaCl, 1 EGTA, 10 HEPES, pH 7.3 adjusted with CsOH; and 3)Bath Solution A: 15 NaCl, 135 N-methyl-D-glucamine-Cl, 1.8 CaCl₂, 0.5 MgCl₂, pH 7.4 with HEPES; or 4) Bath Solution B: 15 NaCl, 135 choline-Cl, 1 MgCl₂, 10 HEPES, pH 7.3 adjusted with TEA-OH. Purified or synthetic peptides are dissolved in 100 mM KCl and 10 mM HEPES-K, pH 7.5, stored at −20° C., and diluted in the bath solution containing 0.05% BSA (essentially fatty acid free, Sigma, St. Louis, Mo.) to prevent adsorption of the peptide to tubing and perfusion chamber. Data are given as mean±standard error.

Tetrodotoxin (TTX)-resistant and TTX-sensitive Na⁺ currents are measured by exposure to appropriate concentrations of TTX and/or by pre-pulse protocols which distinguish between TTX-sensitive and TTX-resistant currents on the basis of their distinct steady-state inactivation properties (Cummins and Waxman, 1997 J. Neurosci. 17:3503-3514; Sontheimer and Waxman, 1992 J. Neurophysiol. 68:1001-1011).

Data are collected using standard pulse protocols and are analyzed to measure sodium current properties that include voltage-dependence, steady-state characteristics, kinetics, and re-priming. Measurements of current amplitude and cell capacitance provides an estimate of Na⁺ current density, thereby permitting comparisons of channel density under different conditions (Cummins and Waxman, 1997 J. Neurosci. 17:3503-3514; Rizzo et al., 1994 J. Neurophysiol. 72:2796-2815). Cells are studied in the current clamp mode to study patterns of spontaneous and evoked action potential generation, threshold for firing, frequency response characteristics, and response to de- and hyperpolarization, and other aspects of electrogenesis (Sontheimer and Waxman, 1992 J. Neurophysiol. 68:1001-1011). These measurements are carried out both in control cells expressing SCN11A or other sodium channel isoforms, and in cells expressing SCN11A or other sodium channel isoforms that also have been exposed to the compound to be tested.

VIPR Assay:

Cells expressing SCN11A or other sodium channel isoforms are plated at approximately 100,000 cells/well in poly-D-lysine coated black-wall clear-bottom 96 well plates (Costar # 3667) and are incubated overnight at 37° C. in a 10% CO₂ atmosphere in growth medium. Cells are stained with voltage-sensitive dyes (described in Gonzalez et al., 1997, Chem. Biol. 4: 269-277; Gonzalez et al., 1995, Biophys. J. 69: 1272-1280) by washing twice with 100 μL of Dulbecco's phosphate buffered saline (DPBS) and then incubating in 100 μL of DPBS supplemented with 10 mM glucose, 10 mM HEPES-Na (pH 7.5), and 10 μM CC2-DMPE for 0.7 hours at 27° C. Cells are rinsed twice with 100 μL of Na-free medium (in mM: 160 tetramethylammonium.Cl, 0.1 CaCl₂, 1 MgCl₂, 11 glucose, 10 HEPES-K, pH 7.5, [K] approximately 4.5 mM) and then incubated in 100 μL of that medium supplemented with 10 μM DiSBAC₂(3), 20 μM veratridine, 20 nM PbTx-3 (brevetoxin), and test sample at the indicated concentration for 0.7 hours at 27° C. At the end of this incubation, the plate is placed in the VIPR reader, illuminated at 400 nm, and fluorescence emissions at 460 and 580 nm are recorded at 1 Hz. After a 7 second baseline reading, 100 μL of Na solution is added (in mM: 165 NaCl, 4.5 KCl, 2 CaCl₂, 1 MgCl₂, 11 glucose, 10 HEPES-Na, pH 7.5). The change in fluorescence resonance energy transfer (FRET) ratio is recorded as F/F₀ or more explicitly as
F/F₀=((S₄₆₀/S₅₈₀)/(I₄₆₀/I₅₈₀))
where S and I are stimulated and initial fluorescence emissions measurements at the indicated wavelengths. The initial second through seventh readings are averaged for the denominator and the stimulated response is picked as the average of the 12th through the 15th readings. Background fluorescence (˜16-20% of the initial signal) is not subtracted.

1. A purified human nucleic acid comprising SEQ ID NO 5, or the complement thereof.

2. The purified nucleic acid of claim 1, wherein said nucleic acid comprises a sequence encoding SEQ ID NO 6.

3. The purified nucleic acid of claim 1, wherein said nucleic acid encodes a polypeptide consisting of SEQ ID NO 6.

4. A purified polypeptide comprising SEQ ID NO 6.

5. The polypeptide of claim 4, wherein said polypeptide consists of SEQ ID NO 6.

6. A method of screening for a compound able to bind to SCN11Asv1 comprising the steps of:
(a) expressing a polypeptide comprising SEQ ID NO 6 from recombinant nucleic acid;
(b) providing to said polypeptide a test preparation comprising one or more test compounds; and
(c) measuring the ability of said test preparation to bind to said polypeptide.

7. The method of claim 6, wherein said steps (b) and (c) are performed in vitro.

8. The method of claim 6, wherein said steps (a), (b), and (c) are performed using a whole cell.

9. The method of claim 6, wherein said polypeptide is expressed from an expression vector comprising a polynucleotide encoding SEQ ID NO 6.

10. A method of screening for compounds able to bind selectively to SCN11Asv1 comprising the steps of:
(a) providing a SCN11Asv1 polypeptide comprising SEQ ID NO 6;
(b) providing one or more sodium channel isoform polypeptides that are not SCN11Asv1;
(c) contacting said SCN11Asv1 polypeptide and said sodium channel isoform polypeptide that is not SCN11Asv1 with a test preparation comprising one or more compounds; and
(d) determining the binding of said test preparation to said SCN11Asv1 polypeptide and to said sodium channel isoform polypeptide that is not SCN11Asv1, wherein a test preparation which binds to said SCN11Asv1 polypeptide, but does not bind to said sodium channel isoform polypeptide that is not SCN11Asv1, contains a compound that selectively binds said SCN11Asv1 polypeptide.

11. The method of claim 10, wherein said SCN11Asv1 polypeptide is obtained by expression of said polypeptide from an expression vector comprising a polynucleotide encoding SEQ ID NO 6.

12. The method of claim 11, wherein said polypeptide consists of SEQ ID NO 6.

13. A method for screening for a compound able to bind to or interact with a SCN11Asv1 protein or a fragment thereof comprising the steps of:
(a) expressing a SCN11Asv1 polypeptide comprising SEQ ID NO 6 or fragment thereof from a recombinant nucleic acid;
(b) providing to said polypeptide a labeled SCN11A ligand that binds to said polypeptide and a test preparation comprising one or more compounds; and
(c) measuring the effect of said test preparation on binding of said labeled SCN11A ligand to said polypeptide, wherein a test preparation that alters the binding of said labeled SCN11A ligand to said polypeptide contains a compound that binds to or interacts with said polypeptide.

14. The method of claim 13, wherein said steps (b) and (c) are performed in vitro.

15. The method of claim 13, wherein said steps (a), (b) and (c) are performed using a whole cell.

16. The method of claim 13, wherein said polypeptide is expressed from an expression vector.

17. The method of claim 13, wherein said SCN11Asv1 ligand is an SCN11A inhibitor.

18. The method of claim 16, wherein said expression vector comprises SEQ ID NO 5 or a fragment of SEQ ID NO 5.

19. The method of claim 16, wherein said polypeptide comprises SEQ ID NO 6 or a fragment of SEQ ID NO 6.

20. A method of screening for SCN11Asv1 activity comprising the steps of:
(a) contacting a cell expressing a recombinant nucleic acid encoding SCN11Asv1 comprising SEQ ID NO 6 with a test preparation comprising one or more test compounds; and
(b) measuring the effect of said test preparation on ion channel activity.


SEQUENCE LISTING (63 sequences)

SEQ ID NO: 1 - DNA, 40 bp, Homo sapiens
aacctgagcctgaacaacagaagtctgatgttaccagtat

SEQ ID NO: 2 - DNA, 40 bp, Homo sapiens
cagtaacccggaagcttatgagtctcttagcagcaaggga

SEQ ID NO: 3 - DNA, 40 bp, Homo sapiens
tttggcttcctatggagaagaccattgctttgaaaagaaa

SEQ ID NO: 4 - DNA, 102 bp, Homo sapiens
agtctcttagcagcaagggaggacttcatccatccagggtgctcctgtgggcagatgcatttcctagagaattgcctcattctttggcttcctatggagaag

SEQ ID NO: 5 - DNA, 5262 bp, Homo sapiens
atggatgacagatgctacccagtaatctttccagatgagcggaatttccgccccttcacttccgactctctggctgcaattgagaagcggattgccatccaaaaggagaaaaagaagtctaaagaccagacaggagaagtaccccagcctcggcctcagcttgacctaaaggcctccaggaagttgcccaagctctatggcgacattcctcgtgagctcataggaaagcctctggaagacttggacccattctaccgaaatcataagacatttatggtgttaaacagaaagaggacaatctaccgcttcagtgccaagcatgccttgttcatttttgggcctttcaattcaatcagaagtttagccattagagtctcagtccattcattgttcagcatgttcattatcggcaccgttatcatcaactgcgtgttcatggctacagggcctgctaaaaacagcaacagtaacaatactgacattgcagagtgtgtcttcactgggatttatatttttgaagctttgattaaaatattggcaagaggtttcattctggatgagttttctttccttcgagatccatggaactggctggactccattgtcattggaatagcgattgtgtcatatattccaggaatcaccatcaaactattgcccctgcgtaccttccgtgtgttcagagctttgaaagcaatttcagtagtttcacgtctgaaggtcatcgtgggggccttgctacgctctgtgaagaagctggtcaacgtgattatcctcaccttcttttgcctcagcatctttgccctggtaggtcagcagctcttcatgggaagtctgaacctgaaatgcatctcgagggactgtaaaaatatcagtaacccggaagcttatgaccattgctttgaaaagaaagaaaattcacctgaattcaaaatgtgtggcatctggatgggtaacagtgcctgttccatacaatatgaatgtaagcacaccaaaattaatcctgactataattatacgaattttgacaactttggctggtcttttcttgccatgttccggctgatgacccaagattcctgggagaagctttatcaacagaccctgcgtactactgggctctactcagtcttcttcttcattgtggtcattttcctgggctccttctacctgattaacttaaccctggctgttgttaccatggcatatgaggagcagaacaagaatgtagctgcagagatagaggccaaggaaaagatgtttcaggaagcccagcagctgttaaaggaggaaaaggaggctctggttgccatgggaattgacagaagttcacttacttcccttgaaacatcatattttaccccaaaaaagagaaagctctttggtaataagaaaaggaagtccttctttttgagagagtctgggaaagaccagcctcctgggtcagattctgatgaagattgccaaaaaaagccacagctcctagagcaaaccaaacgactgtcccagaatctatcactggaccactttgatgagcatggagatcctctccaaaggcagagagcactgagtgctgtcagcatcctcaccatcaccatgaaggaacaagaaaaatcacaagagccttgtctcccttgtggagaaaacctggcatccaagtacctcgtgtggaactgttgcccccagtggctgtgcgttaagaaggtcctgagaactgtgatgactgacccgtttactgagctggccatcaccatctgcatcatcatcaacactgtcttcttggccatggagcatcacaagatggaggccagttttgagaagatgttgaatatagggaatttggttttcactagcatttttatagcagaaatgtgcctaaaaatcattgcgctcgatccctaccactactttcgccgaggctggaacatttttgacagcattgttgctcttctgagttttgcagatgtaatgaactgtgtacttcaaaagagaagctggccattcttgcgttccttcagagtgctcagggtcttcaagttagccaaatcctggccaactttgaacacactaattaagataatcggcaactctgtcggagcccttggaagcctgactgtggtcctggtcattgtgatctttattttctcagtagttggcatgcagctttttggccgtagcttcaattcccaaaagagtccaaaactctgtaacccgacaggcccgacagtctcatgtttacggcactggcacatgggggatttctggcactccttcctagtggtattccgcatcctctgcggggaatggatcgaaaatatgtgggaatgtatgcaagaagcgaatgcatcatcatcattgtgtgttattgtcttcatattgatcacggtgataggaaaacttgtggtgctcaacctcttcattgccttactgctcaattcctttagcaatgaggaaagaaatggaaacttagaaggagaggccaggaaaactaaagtccagttagcactggatcgattccgccgggctttttgttttgtgagacacactcttgagcatttctgtcacaagtggtgcaggaagcaaaacttaccacagcaaaaagaggtggcaggaggctgtgctgcacaaagcaaagacatcattcccctggtcatggagatgaaaaggggctcagagacccaggaggagcttggtatactaacctctgtaccaaagaccctgggcgtcaggcatgattggacttggttggcaccacttgcggaggaggaagatgacgttgaattttctggtgaagataatgcacagcgcatcacacaacctgagcctgaacaacagaagtctgatgttaccagtatactatcagaatgtagcaccattgatcttcaggatggctttggatggttacctgagatggttcccaaaaagcaaccagagagatgtttgcccaaaggctttggttgctgctttccatgctgtagcgtggacaagagaaagcctccctgggtcatttggtggaacctgcggaaaacctgctaccaaatagtgaaacacagctggtttgagagctttattatctttgtgattctgctgagcagtggggcactgatatttgaagatgttcaccttgagaaccaacccaaaatccaagaattactaaattgtactgacattatttttacacatatttttatcctggagatggtactaaaatgggtagccttcggatttggaaagtatttcaccagtgcctggtgctgccttgatttcatcattgtgattgtctctgtgaccaccctcattaacttaatggaattgaagtccttccggactctacgagcactgaggcctcttcgtgcgctgtcccagtttgaaggaatgaaggtggtggtcaatgctctcataggtgccatacctgccattctgaatgttttgcttgtctgcctcattttctggctcgtattttgtattctgggagtatacttcttttctggaaaatttgggaaatgcattaatggaacagactcagttataaattataccatcattacaaataaaagtcaatgtgaaagtggcaatttctcttggatcaaccagaaagtcaactttgacaatgtgggaaatgcttacctcgctctgctgcaagtggcaacatttaagggctggatggatattatatatgcagctgttgattccacagagaaagaacaacagccagagtttgagagcaattcactcggttacatttacttcgtagtctttatcatctttggctcattcttcactctgaatctcttcattggcgttatcattgacaacttcaaccaacagcagaaaaagttaggtggccaagacatttttatgacagaagaacagaagaaatactataatgcaatgaaaaaattaggatccaaaaaacctcaaaaacccattccacggcctctgaacaaatgtcaaggtctcgtgttcgacatagtcacaagccagatctttgacatcatcatcataagtctcattatcctaaacatgattagcatgatggctgaatcatacaaccaacccaaagccatgaaatccatccttgaccatctcaactgggtctttgtggtcatctttacgttagaatgtctcatcaaaatctttgctttgaggcaatactacttcaccaatggctggaatttatttgactgtgtggtcgtgcttctttccattgttagtacaatgatttctaccttggaaaatcaggagcacattcctttccctccgacgctcttcagaattgtccgcttggctcggattggccgaatcctgaggcttgtccgggctgcacgaggaatcaggactctcctctttgctctgatgatgtcgcttccttctctgttcaacattggtcttctactctttctgattatgtttatctatgccattctgggtatgaactggttttccaaagtgaatccagagtctggaatcgatgacatattcaacttcaagacttttgccagcagcatgctctgtctcttccagataagcacatcagcaggttgggattccctgctcagccccatgctgcgatcaaaagaatcatgtaactcttcctcagaaaactgccacctccctggcatagccacatcctactttgtcagttacattatcatctcctttctcattgttgtcaacatgtacattgctgtgattttagagaacttcaatacagccactgaagaaagtgaggaccctttgggtgaagatgactttgacatattttatgaagtgtgggaaaagtttgacccagaagcaacacaatttatcaaatattctgccctttctgactttgctgatgccttgcctgagcctttgcgtgtcgcaaagccaaataaatatcaatttctagtaatggacttgcccatggtgagtgaagatcgcctccactgcatggatattcttttcgccttcaccgctagggtactcggtggctctgatggcctagatagtatgaaagcaatgatggaagagaagttcatggaagccaatcctctcaagaagttgtatgaacccatagtcaccaccaccaagagaaaggaagaggaaagaggtgctgctattattcaaaaggcctttcgaaagtacatgatgaaggtgaccaagggtgaccaaggtgaccaaaatgacttggaaaacgggcctcattcaccactccagactctttgcaatggagacttgtctagctttggggtggccaagggcaaggtccactgtgactga

SEQ ID NO: 6 - protein, 1753 aa, Homo sapiens
MDDRCYPVIFPDERNFRPFTSDSLAAIEKRIAIQKEKKKSKDQTGEVPQPRPQLDLKASRKLPKLYGDIPRELIGKPLEDLDPFYRNHKTFMVLNRKRTIYRFSAKHALFIFGPFNSIRSLAIRVSVHSLFSMFIIGTVIINCVFMATGPAKNSNSNNTDIAECVFTGIYIFEALIKILARGFILDEFSFLRDPWNWLDSIVIGIAIVSYIPGITIKLLPLRTFRVFRALKAISVVSRLKVIVGALLRSVKKLVNVIILTFFCLSIFALVGQQLFMGSLNLKCISRDCKNISNPEAYDHCFEKKENSPEFKMCGIWMGNSACSIQYECKHTKINPDYNYTNFDNFGWSFLAMFRLMTQDSWEKLYQQTLRTTGLYSVFFFIVVIFLGSFYLINLTLAVVTMAYEEQNKNVAAEIEAKEKMFQEAQQLLKEEKEALVAMGIDRSSLTSLETSYFTPKKRKLFGNKKRKSFFLRESGKDQPPGSDSDEDCQKKPQLLEQTKRLSQNLSLDHFDEHGDPLQRQRALSAVSILTITMKEQEKSQEPCLPCGENLASKYLVWNCCPQWLCVKKVLRTVMTDPFTELAITICIIINTVFLAMEHHKMEASFEKMLNIGNLVFTSIFIAEMCLKIIALDPYHYFRRGWNIFDSIVALLSFADVMNCVLQKRSWPFLRSFRVLRVFKLAKSWPTLNTLIKIIGNSVGALGSLTVVLVIVIFIFSVVGMQLFGRSFNSQKSPKLCNPTGPTVSCLRHWHMGDFWHSFLVVFRILCGEWIENMWECMQEANASSSLCVIVFILITVIGKLVVLNLFIALLLNSFSNEERNGNLEGEARKTKVQLALDRFRRAFCFVRHTLEHFCHKWCRKQNLPQQKEVAGGCAAQSKDIIPLVMEMKRGSETQEELGILTSVPKTLGVRHDWTWLAPLAEEEDDVEFSGEDNAQRITQPEPEQQKSDVTSILSECSTIDLQDGFGWLPEMVPKKQPERCLPKGFGCCFPCCSVDKRKPPWVIWWNLRKTCYQIVKHSWFESFIIFVILLSSGALIFEDVHLENQPKIQELLNCTDIIFTHIFILEMVLKWVAFGFGKYFTSAWCCLDFIIVIVSVTTLINLMELKSFRTLRALRPLRALSQFEGMKVVVNALIGAIPAILNVLLVCLIFWLVFCILGVYFFSGKFGKCINGTDSVINYTIITNKSQCESGNFSWINQKVNFDNVGNAYLALLQVATFKGWMDIIYAAVDSTEKEQQPEFESNSLGYIYFVVFIIFGSFFTLNLFIGVIIDNFNQQQKKLGGQDIFMTEEQKKYYNAMKKLGSKKPQKPIPRPLNKCQGLVFDIVTSQIFDIIIISLIILNMISMMAESYNQPKAMKSILDHLNWVFVVIFTLECLIKIFALRQYYFTNGWNLFDCVVVLLSIVSTMISTLENQEHIPFPPTLFRIVRLARIGRILRLVRAARGIRTLLFALMMSLPSLFNIGLLLFLIMFIYAILGMNWFSKVNPESGIDDIFNFKTFASSMLCLFQISTSAGWDSLLSPMLRSKESCNSSSENCHLPGIATSYFVSYIIISFLIVVNMYIAVILENFNTATEESEDPLGEDDFDIFYEVWEKFDPEATQFIKYSALSDFADALPEPLRVAKPNKYQFLVMDLPMVSEDRLHCMDILFAFTARVLGGSDGLDSMKAMMEEKFMEANPLKKLYEPIVTTTKRKEEERGAAIIQKAFRKYMMKVTKGDQGDQNDLENGPHSPLQTLCNGDLSSFGVAKGKVHCD

SEQ ID NO: 7 - DNA, 5475 bp, Homo sapiens
atggatgacagatgctacccagtaatctttccagatgagcggaatttccgccccttcacttccgactctctggctgcaattgagaagcggattgccatccaaaaggagaaaaagaagtctaaagaccagacaggagaagtaccccagcctcggcctcagcttgacctaaaggcctccaggaagttgcccaagctctatggcgacattcctcgtgagctcataggaaagcctctggaagacttggacccattctaccgaaatcataagacatttatggtgttaaacagaaagaggacaatctaccgcttcagtgccaagcatgccttgttcatttttgggcctttcaattcaatcagaagtttagccattagagtctcagtccattcattgttcagcatgttcattatcggcaccgttatcatcaactgcgtgttcatggctacagggcctgctaaaaacagcaacagtaacaatactgacattgcagagtgtgtcttcactgggatttatatttttgaagctttgattaaaatattggcaagaggtttcattctggatgagttttctttccttcgagatccatggaactggctggactccattgtcattggaatagcgattgtgtcatatattccaggaatcaccatcaaactattgcccctgcgtaccttccgtgtgttcagagctttgaaagcaatttcagtagtttcacgtctgaaggtcatcgtgggggccttgctacgctctgtgaagaagctggtcaacgtgattatcctcaccttcttttgcctcagcatctttgccctggtaggtcagcagctcttcatgggaagtctgaacctgaaatgcatctcgagggactgtaaaaatatcagtaacccggaagcttatgagtctcttagcagcaagggaggacttcatccatccagggtgctcctgtgggcagatgcatttcctagagaattgcctcattctttggcttcctatggagaagaccattgctttgaaaagaaagaaaattcacctgaattcaaaatgtgtggcatctggatgggtaacagtgcctgttccatacaatatgaatgtaagcacaccaaaattaatcctgactataattatacgaattttgacaactttggctggtcttttcttgccatgttccggctgatgacccaagattcctgggagaagctttatcaacagaccctgcgtactactgggctctactcagtcttcttcttcattgtggtcattttcctgggctccttctacctgattaacttaaccctggctgttgttaccatggcatatgaggagcagaacaagaatgtagctgcagagatagaggccaaggaaaagatgtttcaggaagcccagcagctgttaaaggaggaaaaggaggctctggttgccatgggaattgacagaagttcacttacttcccttgaaacatcatattttaccccaaaaaagagaaagctctttggtaataagaaaaggaagtccttctttttgagagagtctgggaaagaccagcctcctgggtcagattctgatgaagattgccaaaaaaagccacagctcctagagcaaaccaaacgactgtcccagaatctatcactggaccactttgatgagcatggagatcctctccaaaggcagagagcactgagtgctgtcagcatcctcaccatcaccatgaaggaacaagaaaaatcacaagagccttgtctcccttgtggagaaaacctggcatccaagtacctcgtgtggaactgttgcccccagtggctgtgcgttaagaaggtcctgagaactgtgatgactgacccgtttactgagctggccatcaccatctgcatcatcatcaacactgtcttcttggccatggagcatcacaagatggaggccagttttgagaagatgttgaatatagggaatttggttttcactagcatttttatagcagaaatgtgcctaaaaatcattgcgctcgatccctaccactactttcgccgaggctggaacatttttgacagcattgttgctcttctgagttttgcagatgtaatgaactgtgtacttcaaaagagaagctggccattcttgcgttccttcagagtgctcagggtcttcaagttagccaaatcctggccaactttgaacacactaattaagataatcggcaactctgtcggagcccttggaagcctgactgtggtcctggtcattgtgatctttattttctcagtagttggcatgcagctttttggccgtagcttcaattcccaaaagagtccaaaactctgtaacccgacaggcccgacagtctcatgtttacggcactggcacatgggggatttctggcactccttcctagtggtattccgcatcctctgcggggaatggatcgaaaatatgtgggaatgtatgcaagaagcgaatgcatcatcatcattgtgtgttattgtcttcatattgatcacggtgataggaaaacttgtggtgctcaacctcttcattgccttactgctcaattcctttagcaatgaggaaagaaatggaaacttagaaggagaggccaggaaaactaaagtccagttagcactggatcgattccgccgggctttttgttttgtgagacacactcttgagcatttctgtcacaagtggtgcaggaagcaaaacttaccacagcaaaaagaggtggcaggaggctgtgctgcacaaagcaaagacatcattcccctggtcatggagatgaaaaggggctcagagacccaggaggagcttggtatactaacctctgtaccaaagaccctgggcgtcaggcatgattggacttggttggcaccacttgcggaggaggaagatgacgttgaattttctggtgaagataatgcacagcgcatcacacaacctgagcctgaacaacaggcctatgagctccatcaggagaacaagaagcccacgagccagagagttcaaagtgtggaaattgacatgttctctgaagatgagcctcatctgaccatacaggatccccgaaagaagtctgatgttaccagtatactatcagaatgtagcaccattgatcttcaggatggctttggatggttacctgagatggttcccaaaaagcaaccagagagatgtttgcccaaaggctttggttgctgctttccatgctgtagcgtggacaagagaaagcctccctgggtcatttggtggaacctgcggaaaacctgctaccaaatagtgaaacacagctggtttgagagctttattatctttgtgattctgctgagcagtggggcactgatatttgaagatgttcaccttgagaaccaacccaaaatccaagaattactaaattgtactgacattatttttacacatatttttatcctggagatggtactaaaatgggtagccttcggatttggaaagtatttcaccagtgcctggtgctgccttgatttcatcattgtgattgtctctgtgaccaccctcattaacttaatggaattgaagtccttccggactctacgagcactgaggcctcttcgtgcgctgtcccagtttgaaggaatgaaggtggtggtcaatgctctcataggtgccatacctgccattctgaatgttttgcttgtctgcctcattttctggctcgtattttgtattctgggagtatacttcttttctggaaaatttgggaaatgcattaatggaacagactcagttataaattataccatcattacaaataaaagtcaatgtgaaagtggcaatttctcttggatcaaccagaaagtcaactttgacaatgtgggaaatgcttacctcgctctgctgcaagtggcaacatttaagggctggatggatattatatatgcagctgttgattccacagagaaagaacaacagccagagtttgagagcaattcactcggttacatttacttcgtagtctttatcatctttggctcattcttcactctgaatctcttcattggcgttatcattgacaacttcaaccaacagcagaaaaagttaggtggccaagacatttttatgacagaagaacagaagaaatactataatgcaatgaaaaaattaggatccaaaaaacctcaaaaacccattccacggcctctgaacaaatgtcaaggtctcgtgttcgacatagtcacaagccagatctttgacatcatcatcataagtctcattatcctaaacatgattagcatgatggctgaatcatacaaccaacccaaagccatgaaatccatccttgaccatctcaactgggtctttgtggtcatctttacgttagaatgtctcatcaaaatctttgctttgaggcaatactacttcaccaatggctggaatttatttgactgtgtggtcgtgcttctttccattgttagtacaatgatttctaccttggaaaatcaggagcacattcctttccctccgacgctcttcagaattgtccgcttggctcggattggccgaatcctgaggcttgtccgggctgcacgaggaatcaggactctcctctttgctctgatgatgtcgcttccttctctgttcaacattggtcttctactctttctgattatgtttatctatgccattctgggtatgaactggttttccaaagtgaatccagagtctggaatcgatgacatattcaacttcaagacttttgccagcagcatgctctgtctcttccagataagcacatcagcaggttgggattccctgctcagccccatgctgcgatcaaaagaatcatgtaactcttcctcagaaaactgccacctccctggcatagccacatcctactttgtcagttacattatcatctcctttctcattgttgtcaacatgtacattgctgtgattttagagaacttcaatacagccactgaagaaagtgaggaccctttgggtgaagatgactttgacatattttatgaagtgtgggaaaagtttgacccagaagcaacacaatttatcaaatattctgccctttctgactttgctgatgccttgcctgagcctttgcgtgtcgcaaagccaaataaatatcaatttctagtaatggacttgcccatggtgagtgaagatcgcctccactgcatggatattcttttcgccttcaccgctagggtactcggtggctctgatggcctagatagtatgaaagcaatgatggaagagaagttcatggaagccaatcctctcaagaagttgtatgaacccatagtcaccaccaccaagagaaaggaagaggaaagaggtgctgctattattcaaaaggcctttcgaaagtacatgatgaaggtgaccaagggtgaccaaggtgaccaaaatgacttggaaaacgggcctcattcaccactccagactctttgcaatggagacttgtctagctttggggtggccaagggcaaggtccactgtgac

SEQ ID NO: 8 - protein, 1825 aa, Homo sapiens
MDDRCYPVIFPDERNFRPFTSDSLAAIEKRIAIQKEKKKSKDQTGEVPQPRPQLDLKASRKLPKLYGDIPRELIGKPLEDLDPFYRNHKTFMVLNRKRTIYRFSAKHALFIFGPFNSIRSLAIRVSVHSLFSMFIIGTVIINCVFMATGPAKNSNSNNTDIAECVFTGIYIFEALIKILARGFILDEFSFLRDPWNWLDSIVIGIAIVSYIPGITIKLLPLRTFRVFRALKAISVVSRLKVIVGALLRSVKKLVNVIILTFFCLSIFALVGQQLFMGSLNLKCISRDCKNISNPEAYESLSSKGGLHPSRVLLWADAFPRELPHSLASYGEDHCFEKKENSPEFKMCGIWMGNSACSIQYECKHTKINPDYNYTNFDNFGWSFLAMFRLMTQDSWEKLYQQTLRTTGLYSVFFFIVVIFLGSFYLINLTLAVVTMAYEEQNKNVAAEIEAKEKMFQEAQQLLKEEKEALVAMGIDRSSLTSLETSYFTPKKRKLFGNKKRKSFFLRESGKDQPPGSDSDEDCQKKPQLLEQTKRLSQNLSLDHFDEHGDPLQRQRALSAVSILTITMKEQEKSQEPCLPCGENLASKYLVWNCCPQWLCVKKVLRTVMTDPFTELAITICIIINTVFLAMEHHKMEASFEKMLNIGNLVFTSIFIAEMCLKIIALDPYHYFRRGWNIFDSIVALLSFADVMNCVLQKRSWPFLRSFRVLRVFKLAKSWPTLNTLIKIIGNSVGALGSLTVVLVIVIFIFSVVGMQLFGRSFNSQKSPKLCNPTGPTVSCLRHWHMGDFWHSFLVVFRILCGEWIENMWECMQEANASSSLCVIVFILITVIGKLVVLNLFIALLLNSFSNEERNGNLEGEARKTKVQLALDRFRRAFCFVRHTLEHFCHKWCRKQNLPQQKEVAGGCAAQSKDIIPLVMEMKRGSETQEELGILTSVPKTLGVRHDWTWLAPLAEEEDDVEFSGEDNAQRITQPEPEQQAYELHQENKKPTSQRVQSVEIDMFSEDEPHLTIQDPRKKSDVTSILSECSTIDLQDGFGWLPEMVPKKQPERCLPKGFGCCFPCCSVDKRKPPWVIWWNLRKTCYQIVKHSWFESFIIFVILLSSGALIFEDVHLENQPKIQELLNCTDIIFTHIFILEMVLKWVAFGFGKYFTSAWCCLDFIIVIVSVTTLINLMELKSFRTLRALRPLRALSQFEGMKVVVNALIGAIPAILNVLLVCLIFWLVFCILGVYFFSGKFGKCINGTDSVINYTIITNKSQCESGNFSWINQKVNFDNVGNAYLALLQVATFKGWMDIIYAAVDSTEKEQQPEFESNSLGYIYFVVFIIFGSFFTLNLFIGVIIDNFNQQQKKLGGQDIFMTEEQKKYYNAMKKLGSKKPQKPIPRPLNKCQGLVFDIVTSQIFDIIIISLIILNMISMMAESYNQPKAMKSILDHLNWVFVVIFTLECLIKIFALRQYYFTNGWNLFDCVVVLLSIVSTMISTLENQEHIPFPPTLFRIVRLARIGRILRLVRAARGIRTLLFALMMSLPSLFNIGLLLFLIMFIYAILGMNWFSKVNPESGIDDIFNFKTFASSMLCLFQISTSAGWDSLLSPMLRSKESCNSSSENCHLPGIATSYFVSYIIISFLIVVNMYIAVILENFNTATEESEDPLGEDDFDIFYEVWEKFDPEATQFIKYSALSDFADALPEPLRVAKPNKYQFLVMDLPMVSEDRLHCMDILFAFTARVLGGSDGLDSMKAMMEEKFMEANPLKKLYEPIVTTTKRKEEERGAAIIQKAFRKYMMKVTKGDQGDQNDLENGPHSPLQTLCNGDLSSFGVAKGKVHCD

SEQ ID NO: 9 - DNA, 20 bp, Artificial Sequence
  oligonucleotide probe
tgaacaacagaagtctgatg

SEQ ID NO: 10 - DNA, 20 bp, Artificial Sequence
  oligonucleotide probe
gaagcttatgagtctcttag

SEQ ID NO: 11 - DNA, 20 bp, Artificial Sequence
  oligonucleotide probe
tatggagaagaccattgctt

SEQ ID NO: 12 - protein, 10 aa, Artificial Sequence
  polypeptide
EPEQQKSDVT

SEQ ID NO: 13 - protein, 10 aa, Artificial Sequence
  polypeptide
NPEAYESLSS

SEQ ID NO: 14 - protein, 10 aa, Artificial Sequence
  polypeptide
ASYGEDHCFE

SEQ ID NO: 15 - DNA, 28 bp, Artificial Sequence
  oligonucleotides
gatgacgttgaattttctggtgaagata

SEQ ID NO: 16 - DNA, 27 bp, Artificial Sequence
  oligonucleotides
caaatccgaaggctacccattttagta

SEQ ID NO: 17 - DNA, 65 bp, Artificial Sequence
  oligonucleotide
tatcgaaattaatacgactcactatagggagacccaagctgagggtgaagatggatgacagatgc

SEQ ID NO: 18 - DNA, 28 bp, Artificial Sequence
  oligonucleotide
gtttggtttgctctaggagctgtggctt

SEQ ID NO: 19 - DNA, 28 bp, Artificial Sequence
  oligonucleotide
gttgccatgggaattgacagaagttcac

SEQ ID NO: 20 - DNA, 28 bp, Artificial Sequence
  oligonucleotide
ataggcctgttgttcaggctcaggttgt

SEQ ID NO: 21 - DNA, 28 bp, Artificial Sequence
  oligonucleotide
actaacctctgtaccaaagaccctgggc

SEQ ID NO: 22 - DNA, 29 bp, Artificial Sequence
  oligonucleotide
cgtaaagatgaccacaaagacccagttga

SEQ ID NO: 23 - DNA, 65 bp, Artificial Sequence
  oligonucleotide
aaaatgagaagagttaaacaggtacgtagcatgcccgggcgtgttcgacatagtcacaagccaga

SEQ ID NO: 24 - DNA, 65 bp, Artificial Sequence
  oligonucleotide
gcatattcagatcctcttctgagatgagtttttgttcgaaaaggctgtgaagctatgaggtaggc

SEQ ID NO: 25 - DNA, 64 bp, Artificial Sequence
  oligonucleotide
atacgactcactatagggagacccaagctggctagttaagtatgtttatatatggattttgaaa

SEQ ID NO: 26 - DNA, 41 bp, Artificial Sequence
  oligonucleotide
gtagaggtatggccggtggtgaacatcaccacagaattaac

SEQ ID NO: 27 - DNA, 73 bp, Artificial Sequence
  oligonucleotide
tccaccacactggactagtggatccgagctcggtaccaaggcccgggcatgctacgtacctgtttaactcttc

SEQ ID NO: 28 - DNA, 41 bp, Artificial Sequence
  oligonucleotide
gttaattctgtggtgatgttcaccaccggccatacctctac

SEQ ID NO: 29 - DNA, 80 bp, Artificial Sequence
  linker sequence
tgcgttatcccctgattctgtggataaccgtattaccgctgggtaataactgatataattaaattgaagctctaatttgt

SEQ ID NO: 30 - DNA, 80 bp, Artificial Sequence
  linker sequence
acaaattagagcttcaatttaattatatcagttattacccagcggtaatacggttatccacagaatcaggggataacgca

SEQ ID NO: 31 - DNA, 80 bp, Artificial Sequence
  linker sequence
aatttaaattataattatttttatagcacgtgatgaaaagagcatggatctcggggacgtctaactactaagcgagagta

SEQ ID NO: 32 - DNA, 80 bp, Artificial Sequence
  linker sequence
tactctcgcttagtagttagacgtccccgagatccatgctcttttcatcacgtgctataaaaataattataatttaaatt

SEQ ID NO: 33 - DNA, 4615 bp, Artificial Sequence
  vector
gggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagcgtggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttaggacggatcgcttgcctgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcttctacacagacaagatgaaacaattcggcattaatacctgagagcaggaagagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaagagcatggatctcggggacgtctaactactaagcgagagtagggaactgccaggcatcaaataaaacgaaaggctcagtcggaagactgggcctttcgttttatctgttgtttgtcggtgaacgctctcctgagtaggacaaatccgccgggagcggatttgaacgttgtgaagcaacggcccggagggtggcgggcaggacgcccgccataaactgccaggcatcaaactaagcagaaggccatcctgacggatggcctttttgcgtttctacaaactcttcctgttagttagttacttaagctcgggccccaaataatgattttattttgactgatagtgacctgttcgttgcaacaaattgataagcaatgcttttttataatgccaactttgtacaaaaaagcaggcttcgaaggagatagaaccaattctctaaggaaatacttaaccatggtcgactggatccggtaccgaattcgcttactaaaagccagataacagtatgcgtatttgcgcgctgatttttgcggtataagaatatatactgatatgtatacccgaagtatgtcaaaaagaggtgtgcttctagaatgcagtttaaggtttacacctataaaagagagagccgttatcgtctgtttgtggatgtacagagtgatattattgacacgcccgggcgacggatagtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatatagaattcgcggccgcactcgagatatctagacccagctttcttgtacaaagttggcattataagaaagcattgcttatcaatttgttgcaacgaacaggtcactatcagtcaaaataaaatcattatttgccatccagctgcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacattattcagattgggccccgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgct

SEQ ID NO: 34 - DNA, 88 bp, Artificial Sequence
  linker
actttgtacaaaaaagcaggcttcgaaggagatagaaccagcccgggcgccgcactcgagatatctagacccagctttcttgtacaaa

SEQ ID NO: 35 - DNA, 88 bp, Artificial Sequence
  linker
tttgtacaagaaagctgggtctagatatctcgagtgcggcgcccgggctggttctatctccttcgaagcctgcttttttgtacaaagt

SEQ ID NO: 36 - DNA, 4146 bp, Artificial Sequence
  vector
gggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagcgtggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttaggacggatcgcttgcctgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcttctacacagacaagatgaaacaattcggcattaatacctgagagcaggaagagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaagagcatggatctcggggacgtctaactactaagcgagagtagggaactgccaggcatcaaataaaacgaaaggctcagtcggaagactgggcctttcgttttatctgttgtttgtcggtgaacgctctcctgagtaggacaaatccgccgggagcggatttgaacgttgtgaagcaacggcccggagggtggcgggcaggacgcccgccataaactgccaggcatcaaactaagcagaaggccatcctgacggatggcctttttgcgtttctacaaactcttcctgttagttagttacttaagctcgggccccaaataatgattttattttgactgatagtgacctgttcgttgcaacaaattgataagcaatgcttttttataatgccaactttgtacaaaaaagcaggcttcgaaggagatagaaccagcccgggcgccgcactcgagatatctagacccagctttcttgtacaaagttggcattataagaaagcattgcttatcaatttgttgcaacgaacaggtcactatcagtcaaaataaaatcattatttgccatccagctgcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacattattcagattgggccccgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgct

SEQ ID NO: 37 - DNA, 80 bp, Artificial Sequence
  linker
gttaaccgggctgcatccgatgcaagtgtgtcgctgtcgagggtaataactgatataattaaattgaagctctaatttgt

SEQ ID NO: 38 - DNA, 80 bp, Artificial Sequence
  linker
acaaattagagcttcaatttaattatatcagttattaccctcgacagcgacacacttgcatcggatgcagcccggttaac

SEQ ID NO: 39 - DNA, 80 bp, Artificial Sequence
  linker
ataaaatcattatttgccatccagctgcagctctggcccgtcgaatttctgccattcatccgcttattatcacttattca

SEQ ID NO: 40 - DNA, 80 bp, Artificial Sequence
  linker
tgaataagtgataataagcggatgaatggcagaaattcgacgggccagagctgcagctggatggcaaataatgattttat

SEQ ID NO: 41 - DNA, 9430 bp, Artificial Sequence
  vector
gggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagcgtggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttaggacggatcgcttgcctgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcttctacacagacaagatgaaacaattcggcattaatacctgagagcaggaagagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaagagcatggatctcggggacgtctaactactaagcgagagtagggaactgccaggcatcaaataaaacgaaaggctcagtcggaagactgggcctttcgttttatctgttgtttgtcggtgaacgctctcctgagtaggacaaatccgccgggagcggatttgaacgttgtgaagcaacggcccggagggtggcgggcaggacgcccgccataaactgccaggcatcaaactaagcagaaggccatcctgacggatggcctttttgcgtttctacaaactcttcctgttagttagttacttaagctcgggccccaaataatgattttattttgactgatagtgacctgttcgttgcaacaaattgataagcaatgcttttttataatgccaactttgtacaaaaaagcaggcttcgaaggagatagaaccagcccgggcgccgcactcgagatatctagacccagctttcttgtacaaagttggcattataagaaagcattgcttatcaatttgttgcaacgaacaggtcactatcagtcaaaataaaatcattatttgccatccagctgcagctctggcccgtcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcggctggttgccctcgccgctgggctggcggccgtctatggccctgcaaacgcgccagaaacgccgtcgaagccgtgtgcgagacaccgcggccggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggtcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagtaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgtcga

SEQ ID NO: 42 - DNA, 80 bp, Artificial Sequence
  linker
acggttatccacagaatcaggggataacgcaggaaagaacataaaatcattatttgccatccagctgcagctctggcccg

SEQ ID NO: 43 - DNA, 80 bp, Artificial Sequence
  linker
cgggccagagctgcagctggatggcaaataatgattttatgttctttcctgcgttatcccctgattctgtggataaccgt

SEQ ID NO: 44 - DNA, 80 bp, Artificial Sequence
  linker
aatttaaattataattatttttatagcacgtgatgaaaagtccgcgcacatttccccgaaaagtgccacctgacgtcgac

SEQ ID NO: 45 - DNA, 80 bp, Artificial Sequence
  linker
gtcgacgtcaggtggcacttttcggggaaatgtgcgcggacttttcatcacgtgctataaaaataattataatttaaatt

SEQ ID NO: 46 - DNA, 12604 bp, Artificial Sequence
  vector
gggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagcgtggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttaggacggatcgcttgcctgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcttctacacagacaagatgaaacaattcggcattaatacctgagagcaggaagagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaagtccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagttaagcttggtaccgagctcggatccactagtccagtgtggtggaattctgcagatatccagcacagtggcggccgctcgagtctagagggcccttcgaacaaaaactcatctcagaagaggatctgaatatgcataccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacataaaatcattatttgccatccagctgcagctctggcccgtcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcggctggttgccctcgccgctgggctggcggccgtctatggccctgcaaacgcgccagaaacgccgtcgaagccgtgtgcgagacaccgcggccggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggtcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagtaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgtcga

SEQ ID NO: 47 - DNA, 41 bp, Artificial Sequence
  oligonucleotides
gtagaggtatggccggtggtcaacatcaccacagaattaac

SEQ ID NO: 48 - DNA, 41 bp, Artificial Sequence
  oligonucleotides
gttaattctgtggtgatgttgaccaccggccatacctctac

SEQ ID NO: 49 - DNA, 14561 bp, Artificial Sequence
  vector
catggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcggctggttgccctcgccgctgggctggcggccgtctatggccctgcaaacgcgccagaaacgccgtcgaagccgtgtgcgagacaccgcggccggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggtcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagtaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgtcgagggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagcgtggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttaggacggatcgcttgcctgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcttctacacagacaagatgaaacaattcggcattaatacctgagagcaggaagagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaagtccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagttaagtatgtttatatatggattttgaaaatcttttaaaaaagcgataaaagcgttgggatctgccactggtaacttcaaaatagacaagacgaaaaaacggtgaaaatgggtgatagaaataatacagaagtagatgttgaattagattaaactgaagatatataatttattggaaaatacatagagtttttgttgatgcgcttaagcgatcaattcaacaacaccaccagcagctctgattttttcttcagccaacttggagacgaatctagctttgacgataactggaacatttggaattctacccttacccaagatcttaccgtaaccggctgccaaagtgtcaataactggagcagtttccttagaagcagatttcaagtattggtctctcttgtcttctgggatcaatgtccacaatttgtccaagttcaagactggcttccagaaatgagcttgttgcttgtggaagtatctcataccaaccttaccgaaataacctggatggtatttatccatgttaattctgtggtgatgttcaccaccggccatacctctaccaccggggtgctttctgtgcttaccgatacgacctttaccggctgtacaaaaaaaatattgtaatgaaatacaatttttaaatcgttagtaaaacaggttttcaactggacattttatcgaaacaaaagaaacgtggtaactctttttcataattgcgctctctacaaccatttgatacagcgataattagtgctttccataatctaaatgaaagttaccttttatagccgtaaaatgagtagacagtatgactgttctgaatatcatggtataataaaggagaagtatgacaaaggctatgcacactttctttttgctcagtttgcgatggaagagtacatttactcgagctattacctcaaataagccgtctcaacagtgagatggtaatacaaagttcgcaatcaaaacacaaagaaatatgttgagtacacttagctgattgacattctcctgtaccatgatatacacacgacatattggttgcacaacactaaaaaaaaagtcaccgtcttggttctttcattccctcttccaaatggaactacatacctgagacgtgacctctgtgctttctagtcttagtgaatctggaaggcattcttgattagttggatgattgttctgggatttaatgcaaaaaaatcactaagaaggaaaaaaatcaacggagaaagcaaacgccatcttaaatatacgggatacagatgaaaggtttgaacctatctgggaaaatacgcattaaacaagcgaaaaactgcgaggaaaattgtttgcgtctctgcgggctattcacgcgccagaggaaaaataggaaaaataacaggcattagaaaaataattttgattttggtaatgtgtgggtccctggtgtacagatgttacattggttacagtactcttgtttttgctgtgtttttcgaaagggtttcctgggaactagtgctataatgtcaacatcaagtatcgtagatttcaattcaacttagctcttattacagtttatatataatacgcatttcataaggttcattccacctcgtctcttgtaatcattacaatgctgttgatcaactatatttaacaatattatagcttgtttctttataacattagcttacgctcataggcggcagtaaacgatgattaaccatgaaacgtaaggtaacggtggcataatttggtatttgttttctttttttcattttttgaactattacttggttttttcgagagcaaactacctcatacttatttcttgtgtgttaatattatattggcggcaaagtgaaaggggtgatcattctgttatgaaaaaaataaagaacttaagaggaacggtgaagaaagaaaacggataaaatgagaagagttaaacaggtacgtagcatgcccgggccttggtaccgagctcggatccactagtccagtgtggtggaattctgcagatatccagcacagtggcggccgctcgagtctagagggcccttcgaacaaaaactcatctcagaagaggatctgaatatgcataccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacataaaatcattatttgccatccagctgcagctctggcccgtcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagct

SEQ ID NO: 50 - DNA, 65 bp, Artificial Sequence
  oligonucleotides
ataaaatcattatttgccatccagctgcagctctggcccgtgtctcaaaatctctgatgttacat

SEQ ID NO: 51 - DNA, 65 bp, Artificial Sequence
  oligonucleotides
tttctctgtccttcctgtgcgacggttacgccgctccatggtctgacgctcagtggaacggggcc

SEQ ID NO: 52 - DNA, 14567 bp, Artificial Sequence
  vector
catggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcggctggttgccctcgccgctgggctggcggccgtctatggccctgcaaacgcgccagaaacgccgtcgaagccgtgtgcgagacaccgcggccggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggtcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagtaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgtcgagggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagcgtggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttaggacggatcgcttgcctgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcttctacacagacaagatgaaacaattcggcattaatacctgagagcaggaagagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaagtccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagttaagtatgtttatatatggattttgaaaatcttttaaaaaagcgataaaagcgttgggatctgccactggtaacttcaaaatagacaagacgaaaaaacggtgaaaatgggtgatagaaataatacagaagtagatgttgaattagattaaactgaagatatataatttattggaaaatacatagagtttttgttgatgcgcttaagcgatcaattcaacaacaccaccagcagctctgattttttcttcagccaacttggagacgaatctagctttgacgataactggaacatttggaattctacccttacccaagatcttaccgtaaccggctgccaaagtgtcaataactggagcagtttccttagaagcagatttcaagtattggtctctcttgtcttctgggatcaatgtccacaatttgtccaagttcaagactggcttccagaaatgagcttgttgcttgtggaagtatctcataccaaccttaccgaaataacctggatggtatttatccatgttaattctgtggtgatgttcaccaccggccatacctctaccaccggggtgctttctgtgcttaccgatacgacctttaccggctgtacaaaaaaaatattgtaatgaaatacaatttttaaatcgttagtaaaacaggttttcaactggacattttatcgaaacaaaagaaacgtggtaactctttttcataattgcgctctctacaaccatttgatacagcgataattagtgctttccataatctaaatgaaagttaccttttatagccgtaaaatgagtagacagtatgactgttctgaatatcatggtataataaaggagaagtatgacaaaggctatgcacactttctttttgctcagtttgcgatggaagagtacatttactcgagctattacctcaaataagccgtctcaacagtgagatggtaatacaaagttcgcaatcaaaacacaaagaaatatgttgagtacacttagctgattgacattctcctgtaccatgatatacacacgacatattggttgcacaacactaaaaaaaaagtcaccgtcttggttctttcattccctcttccaaatggaactacatacctgagacgtgacctctgtgctttctagtcttagtgaatctggaaggcattcttgattagttggatgattgttctgggatttaatgcaaaaaaatcactaagaaggaaaaaaatcaacggagaaagcaaacgccatcttaaatatacgggatacagatgaaaggtttgaacctatctgggaaaatacgcattaaacaagcgaaaaactgcgaggaaaattgtttgcgtctctgcgggctattcacgcgccagaggaaaaataggaaaaataacaggcattagaaaaataattttgattttggtaatgtgtgggtccctggtgtacagatgttacattggttacagtactcttgtttttgctgtgtttttcgaaagggtttcctgggaactagtgctataatgtcaacatcaagtatcgtagatttcaattcaacttagctcttattacagtttatatataatacgcatttcataaggttcattccacctcgtctcttgtaatcattacaatgctgttgatcaactatatttaacaatattatagcttgtttctttataacattagcttacgctcataggcggcagtaaacgatgattaaccatgaaacgtaaggtaacggtggcataatttggtatttgttttctttttttcattttttgaactattacttggttttttcgagagcaaactacctcatacttatttcttgtgtgttaatattatattggcggcaaagtgaaaggggtgatcattctgttatgaaaaaaataaagaacttaagaggaacggtgaagaaagaaaacggataaaatgagaagagttaaacaggtacgtagcatgcccgggccttggtaccgagctcggatccactagtccagtgtggtggaattctgcagatatccagcacagtggcggccgctcgagtctagagggcccttcgaacaaaaactcatctcagaagaggatctgaatatgcataccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacataaaatcattatttgccatccagctgcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacattattcagattgggccccgttccactgagcgtcagac

SEQ ID NO: 53 - DNA, 362 bp, Artificial Sequence
  restriction enzyme digested DNA fragment
gaattcgcccttgatgacgttgaattttctggtgaagataatgcacagcgcatcacacaacctgagcctgaacaacagaagtctgatgttaccagtatactatcagaatgtagcaccattgatcttcaggatggctttggatggttacctgagatggttcccaaaaagcaaccagagagatgtttgcccaaaggctttggttgctgctttccatgctgtagcgtggacaagagaaagcctccctgggtcatttggtggaacctgcggaaaacctgctaccaaatagtgaaacacagctggtttgagagctttattatctttgtgattctgctgagcagtggggcactgatatttgaagatgt

SEQ ID NO: 54 - DNA, 17815 bp, Artificial Sequence
  vector
gggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagcgtggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttaggacggatcgcttgcctgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcttctacacagacaagatgaaacaattcggcattaatacctgagagcaggaagagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaagtccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctgagggtgaagatggatgacagatgctacccagtaatctttccagatgagcggaatttccgccccttcacttccgactctctggctgcaattgagaagcggattgccatccaaaaggagaaaaagaagtctaaagaccagacaggagaagtaccccagcctcggcctcagcttgacctaaaggcctccaggaagttgcccaagctctatggcgacattcctcgtgagctcataggaaagcctctggaagacttggacccattctaccgaaatcataagacatttatggtgttaaacagaaagaggacaatctaccgcttcagtgccaagcatgccttgttcatttttgggcctttcaattcaatcagaagtttagccattagagtctcagtccattcattgttcagcatgttcattatcggcaccgttatcatcaactgcgtgttcatggctacagggcctgctaaaaacagcaacagtaacaatactgacattgcagagtgtgtcttcactgggatttatatttttgaagctttgattaaaatattggcaagaggtttcattctggatgagttttctttccttcgagatccatggaactggctggactccattgtcattggaatagcgattgtgtcatatattccaggaatcaccatcaaactattgcccctgcgtaccttccgtgtgttcagagctttgaaagcaatttcagtagtttcacgtctgaaggtcatcgtgggggccttgctacgctctgtgaagaagctggtcaacgtgattatcctcaccttcttttgcctcagcatctttgccctggtaggtcagcagctcttcatgggaagtctgaacctgaaatgcatctcgagggactgtaaaaatatcagtaacccggaagcttatgaccattgctttgaaaagaaagaaaattcacctgaattcaaaatgtgtggcatctggatgggtaacagtgcctgttccatacaatatgaatgtaagcacaccaaaattaatcctgactataattatacgaattttgacaactttggctggtcttttcttgccatgttccggctgatgacccaagattcctgggagaagctttatcaacagaccctgcgtactactgggctctactcagtcttcttcttcattgtggtcattttcctgggctccttctacctgattaacttaaccctggctgttgttaccatggcatatgaggagcagaacaagaatgtagctgcagagatagaggccaaggaaaagatgtttcaggaagcccagcagctgttaaaggaggaaaaggaggctctggttgccatgggaattgacagaagttcacttacttcccttgaaacatcatattttaccccaaaaaagagaaagctctttggtaataagaaaaggaagtccttctttttgagagagtctgggaaagaccagcctcctgggtcagattctgatgaagattgccaaaaaaagccacagctcctagagcaaaccaaacgactgtcccagaatctatcactggaccactttgatgagcatggagatcctctccaaaggcagagagcactgagtgctgtcagcatcctcaccatcaccatgaaggaacaagaaaaatcacaagagccttgtctcccttgcggagaaaacctggcatccaagtacctcgtgtggaactgttgcccccagtggctgtgcgttaagaaggtcctgagaactgtgatgactgacccgtttactgagctggccatcaccatctgcatcatcatcaacactgtcttcttggccatggagcatcacaagatggaggccagttttgagaagatgttgaatatagggaatttggttttcactagcatttttatagcagaaatgtgcctaaaaatcattgcgctcgatccctaccactactttcgccgaggctggaacatttttgacagcattgttgctcttctgagttttgcagatgtaatgaactgtgtacttcaaaagagaagctggccattcttgcgttccttcagagtgctcagggtcttcaagttagccaaatcctggccaactttgaacacactaattaagataatcggcaactctgtcggagcccttggaagcctgactgtggtcctggtcattgtgatctttattttctcagtagttggcatgcagctttttggccgtagcttcaattcccaaaagagtccaaaactctgtaacccgacaggcccgacagtctcatgtttacggcactggcacatgggggatttctggcactccttcctagtggtattccgcatcctctgcggggaatggatcgaaaatatgtgggaatgtatgcaagaagcgaatgcatcatcatcattgtgtgttattgtcttcatattgatcacggtgataggaaaacttgtggtgctcaacctcttcattgccttactgctcaattcctttagcaatgaggaaagaaatggaaacttagaaggagaggccaggaaaactaaagtccagttagcactggatcgattccgccgggctttttgttttgtgagacacactcttgagcatttctgtcacaagtggtgcaggaagcaaaacttaccacagcaaaaagaggtggcaggaggctgtgctgcacaaagcaaagacatcattcccctggtcatggagatgaaaaggggctcagagacccaggaggagcttggtatactaacctctgtaccaaagaccctgggcgtcaggcatgattggacttggttggcaccacttgcggaggaggaagatgacgttgaattttctggtgaagataatgcacagcgcatcacacaacctgagcctgaacaacagaagtctgatgttaccagtatactatcagaatgtagcaccattgatcttcaggatggctttggatggttacctgagatggttcccaaaaagcaaccagagagatgtttgcccaaaggctttggttgctgctttccatgctgtagcgtggacaagagaaagcctccctgggtcatttggtggaacctgcggaaaacctgctaccaaatagtgaaacacagctggtttgagagctttattatctttgtgattctgctgagcagtggggcactgatatttgaagatgttcaccttgagaaccaacccaaaatccaagaattactaaattgtactgacattatttttacacatatttttatcctggagatggtactaaaatgggtagccttcggatttggaaagtatttcaccagtgcctggtgctgccttgatttcatcattgtgattgtctctgtgaccaccctcattaacttaatggaattgaagtccttccggactctacgagcactgaggcctcttcgtgcgctgtcccagtttgaaggaatgaaggtggtggtcaatgctctcataggtgccatacctgccattctgaatgttttgcttgtctgcctcattttctggctcgtattttgtattctgggagtatacttcttttctggaaaatttgggaaatgcattaatggaacagactcagttataaattataccatcattacaaataaaagtcaatgtgaaagtggcaatttctcttggatcaaccagaaagtcaactttgacaatgtgggaaatgcttacctcgctctgctgcaagtggcaacatttaagggctggatggatattatatatgcagctgttgattccacagagaaagaacaacagccagagtttgagagcaattcactcggttacatttacttcgtagtctttatcatctttggctcattcttcactctgaatctcttcattggcgttatcattgacaacttcaaccaacagcagaaaaagttaggtggccaagacatttttatgacagaagaacagaagaaatactataatgcaatgaaaaaattaggatccaaaaaacctcaaaaacccattccacggcctctgaacaaatgtcaaggtctcgtgttcgacatagtcacaagccagatctttgacatcatcatcataagtctcattatcctaaacatgattagcatgatggctgaatcatacaaccaacccaaagccatgaaatccatccttgaccatctcaactgggtctttgtggtcatctttacgttagaatgtctcatcaaaatctttgctttgaggcaatactacttcaccaatggctggaatttatttgactgtgtggtcgtgcttctttccattgttagtacaatgatttctaccttggaaaatcaggagcacattcctttccctccgacgctcttcagaattgtccgcttggctcggattggccgaatcctgaggcttgtccgggctgcacgaggaatcaggactctcctctttgctctgatgatgtcgcttccttctctgttcaacattggtcttctactctttctgattatgtttatctatgccattctgggtatgaactggttttccaaagtgaatccagagtctggaatcgatgacatattcaacttcaagacttttgccagcagcatgctctgtctcttccagataagcacatcagcaggttgggattccctgctcagccccatgctgcgatcaaaagaatcatgtaactcttcctcagaaaactgccacctccctggcatagccacatcctactttgtcagttacattatcatctcctttctcattgttgtcaacatgtacattgctgtgattttagagaacttcaatacagccactgaagaaagtgaggaccctttgggtgaagatgactttgacatattttatgaagtgtgggaaaagtttgacccagaagcaacacaatttatcaaatattctgccctttctgactttgctgatgccttgcctgagcctttgcgtgtcgcaaagccaaataaatatcaatttctagtaatggacttgcccatggtgagtgaagatcgcctccactgcatggatattcttttcgccttcaccgctagggtactcggtggctctgatggcctagatagtatgaaagcaatgatggaagagaagttcatggaagccaatcctctcaagaagttgtatgaacccatagtcaccaccaccaagagaaaggaagaggaaagaggtgctgctattattcaaaaggcctttcgaaagtacatgatgaaggtgaccaagggtgaccaaggtgaccaaaatgacttggaaaacgggcctcattcaccactccagactctttgcaatggagacttgtctagctttggggtggccaagggcaaggtccactgtgactgagccctcacctccacgcctacctcatagcttcacagccttttcgaacaaaaactcatctcagaagaggatctgaatatgcataccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacataaaatcattatttgccatccagctgcagctctggcccgtcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcggctggttgccctcgccgctgggctggcggccgtctatggccctgcaaacgcgccagaaacgccgtcgaagccgtgtgcgagacaccgcggccggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggtcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagtaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgtcga

SEQ ID NO: 55 - DNA, 65 bp, Artificial Sequence; identical to SEQ ID NO: 17
  oligonucleotides

SEQ ID NO: 56 - DNA, 40 bp, Artificial Sequence
  oligonucleotides
ccattgctttgaaaagaaagaaaattcacctgaattcaaa

SEQ ID NO: 57 - DNA, 18031 bp, Artificial Sequence
  vector
gggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagcgtggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttaggacggatcgcttgcctgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcttctacacagacaagatgaaacaattcggcattaatacctgagagcaggaagagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaagtccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctgagggtgaagatggatgacagatgctacccagtaatctttccagatgagcggaatttccgccccttcacttccgactctctggctgcaattgagaagcggattgccatccaaaaggagaaaaagaagtctaaagaccagacaggagaagtaccccagcctcggcctcagcttgacctaaaggcctccaggaagttgcccaagctctatggcgacattcctcgtgagctcataggaaagcctctggaagacttggacccattctaccgaaatcataagacatttatggtgttaaacagaaagaggacaatctaccgcttcagtgccaagcatgccttgttcatttttgggcctttcaattcaatcagaagtttagccattagagtctcagtccattcattgttcagcatgttcattatcggcaccgttatcatcaactgcgtgttcatggctacagggcctgctaaaaacagcaacagtaacaatactgacattgcagagtgtgtcttcactgggatttatatttttgaagctttgattaaaatattggcaagaggtttcattctggatgagttttctttccttcgagatccatggaactggctggactccattgtcattggaatagcgattgtgtcatatattccaggaatcaccatcaaactattgcccctgcgtaccttccgtgtgttcagagctttgaaagcaatttcagtagtttcacgtctgaaggtcatcgtgggggccttgctacgctctgtgaagaagctggtcaacgtgattatcctcaccttcttttgcctcagcatctttgccctggtaggtcagcagctcttcatgggaagtctgaacctgaaatgcatctcgagggactgtaaaaatatcagtaacccggaagcttatgagtctcttagcagcaagggaggacttcatccatccagggtgctcctgtgggcagatgcatttcctagagaattgcctcattctttggcttcctatggagaagaccattgctttgaaaagaaagaaaattcacctgaattcaaaatgtgtggcatctggatgggtaacagtgcctgttccatacaatatgaatgtaagcacaccaaaattaatcctgactataattatacgaattttgacaactttggctggtcttttcttgccatgttccggctgatgacccaagattcctgggagaagctttatcaacagaccctgcgtactactgggctctactcagtcttcttcttcattgtggtcattttcctgggctccttctacctgattaacttaaccctggctgttgttaccatggcatatgaggagcagaacaagaatgtagctgcagagatagaggccaaggaaaagatgtttcaggaagcccagcagctgttaaaggaggaaaaggaggctctggttgccatgggaattgacagaagttcacttacttcccttgaaacatcatattttaccccaaaaaagagaaagctctttggtaataagaaaaggaagtccttctttttgagagagtctgggaaagaccagcctcctgggtcagattctgatgaagattgccaaaaaaagccacagctcctagagcaaaccaaacgactgtcccagaatctatcactggaccactttgatgagcatggagatcctctccaaaggcagagagcactgagtgctgtcagcatcctcaccatcaccatgaaggaacaagaaaaatcacaagagccttgtctcccttgcggagaaaacctggcatccaagtacctcgtgtggaactgttgcccccagtggctgtgcgttaagaaggtcctgagaactgtgatgactgacccgtttactgagctggccatcaccatctgcatcatcatcaacactgtcttcttggccatggagcatcacaagatggaggccagttttgagaagatgttgaatatagggaatttggttttcactagcatttttatagcagaaatgtgcctaaaaatcattgcgctcgatccctaccactactttcgccgaggctggaacatttttgacagcattgttgctcttctgagttttgcagatgtaatgaactgtgtacttcaaaagagaagctggccattcttgcgttccttcagagtgctcagggtcttcaagttagccaaatcctggccaactttgaacacactaattaagataatcggcaactctgtcggagcccttggaagcctgactgtggtcctggtcattgtgatctttattttctcagtagttggcatgcagctttttggccgtagcttcaattcccaaaagagtccaaaactctgtaacccgacaggcccgacagtctcatgtttacggcactggcacatgggggatttctggcactccttcctagtggtattccgcatcctctgcggggaatggatcgaaaatatgtgggaatgtatgcaagaagcgaatgcatcatcatcattgtgtgttattgtcttcatattgatcacggtgataggaaaacttgtggtgctcaacctcttcattgccttactgctcaattcctttagcaatgaggaaagaaatggaaacttagaaggagaggccaggaaaactaaagtccagttagcactggatcgattccgccgggctttttgttttgtgagacacactcttgagcatttctgtcacaagtggtgcaggaagcaaaacttaccacagcaaaaagaggtggcaggaggctgtgctgcacaaagcaaagacatcattcccctggtcatggagatgaaaaggggctcagagacccaggaggagcttggtatactaacctctgtaccaaagaccctgggcgtcaggcatgattggacttggttggcaccacttgcggaggaggaagatgacgttgaattttctggtgaagataatgcacagcgcatcacacaacctgagcctgaacaacaggcctatgagctccatcaggagaacaagaagcccacgagccagagagttcaaagtgtggaaattgacatgttctctgaagatgagcctcatctgaccatacaggatccccgaaagaagtctgatgttaccagtatactatcagaatgtagcaccattgatcttcaggatggctttggatggttacctgagatggttcccaaaaagcaaccagagagatgtttgcccaaaggctttggttgctgctttccatgctgtagcgtggacaagagaaagcctccctgggtcatttggtggaacctgcggaaaacctgctaccaaatagtgaaacacagctggtttgagagctttattatctttgtgattctgctgagcagtggggcactgatatttgaagatgttcaccttgagaaccaacccaaaatccaagaattactaaattgtactgacattatttttacacatatttttatcctggagatggtactaaaatgggtagccttcggatttggaaagtatttcaccagtgcctggtgctgccttgatttcatcattgtgattgtctctgtgaccaccctcattaacttaatggaattgaagtccttccggactctacgagcactgaggcctcttcgtgcgctgtcccagtttgaaggaatgaaggtggtggtcaatgctctcataggtgccatacctgccattctgaatgttttgcttgtctgcctcattttctggctcgtattttgtattctgggagtatacttcttttctggaaaatttgggaaatgcattaatggaacagactcagttataaattataccatcattacaaataaaagtcaatgtgaaagtggcaatttctcttggatcaaccagaaagtcaactttgacaatgtgggaaatgcttacctcgctctgctgcaagtggcaacatttaagggctggatggatattatatatgcagctgttgattccacagagaaagaacaacagccagagtttgagagcaattcactcggttacatttacttcgtagtctttatcatctttggctcattcttcactctgaatctcttcattggcgttatcattgacaacttcaaccaacagcagaaaaagttaggtggccaagacatttttatgacagaagaacagaagaaatactataatgcaatgaaaaaattaggatccaaaaaacctcaaaaacccattccacggcctctgaacaaatgtcaaggtctcgtgttcgacatagtcacaagccagatctttgacatcatcatcataagtctcattatcctaaacatgattagcatgatggctgaatcatacaaccaacccaaagccatgaaatccatccttgaccatctcaactgggtctttgtggtcatctttacgttagaatgtctcatcaaaatctttgctttgaggcaatactacttcaccaatggctggaatttatttgactgtgtggtcgtgcttctttccattgttagtacaatgatttctaccttggaaaatcaggagcacattcctttccctccgacgctcttcagaattgtccgcttggctcggattggccgaatcctgaggcttgtccgggctgcacgaggaatcaggactctcctctttgctctgatgatgtcgcttccttctctgttcaacattggtcttctactctttctgattatgtttatctatgccattctgggtatgaactggttttccaaagtgaatccagagtctggaatcgatgacatattcaacttcaagacttttgccagcagcatgctctgtctcttccagataagcacatcagcaggttgggattccctgctcagccccatgctgcgatcaaaagaatcatgtaactcttcctcagaaaactgccacctccctggcatagccacatcctactttgtcagttacattatcatctcctttctcattgttgtcaacatgtacattgctgtgattttagagaacttcaatacagccactgaagaaagtgaggaccctttgggtgaagatgactttgacatattttatgaagtgtgggaaaagtttgacccagaagcaacacaatttatcaaatattctgccctttctgactttgctgatgccttgcctgagcctttgcgtgtcgcaaagccaaataaatatcaatttctagtaatggacttgcccatggtgagtgaagatcgcctccactgcatggatattcttttcgccttcaccgctagggtactcggtggctctgatggcctagatagtatgaaagcaatgatggaagagaagttcatggaagccaatcctctcaagaagttgtatgaacccatagtcaccaccaccaagagaaaggaagaggaaagaggtgctgctattattcaaaaggcctttcgaaagtacatgatgaaggtgaccaagggtgaccaaggtgaccaaaatgacttggaaaacgggcctcattcaccactccagactctttgcaatggagacttgtctagctttggggtggccaagggcaaggtccactgtgactgagccctcacctccacgcctacctcatagcttcacagccttttcgaacaaaaactcatctcagaagaggatctgaatatgcataccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacataaaatcattatttgccatccagctgcagctctggcccgtcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcggctggttgccctcgccgctgggctggcggccgtctatggccctgcaaacgcgccagaaacgccgtcgaagccgtgtgcgagacaccgcggccggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggtcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagtaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgtcga

SEQ ID NO: 58 - DNA, 21 bp, Artificial Sequence
  oligonucleotides
tcacacaacctgagcctgaac

SEQ ID NO: 59 - DNA, 22 bp, Artificial Sequence
  oligonucleotides
gtgggcttcttgttctcctgat

SEQ ID NO: 60 - DNA, 18 bp, Artificial Sequence
  oligonucleotide probe
aacaggcctatgagctcc

SEQ ID NO: 61 - DNA, 19 bp, Artificial Sequence
  oligonucleotides
acagcgcatcacacaacct

SEQ ID NO: 62 - DNA, 26 bp, Artificial Sequence
  oligonucleotides
ctgaagatcaatggtgctacattctg

SEQ ID NO: 63 - DNA, 18 bp, Artificial Sequence
  oligonucleotide probe
cctgaacaacagaagtct